SEQUENCE LISTING (132 sequences)

SEQ ID NO: 1 - protein, 119 aa, Artificial Sequence
  Description of Artificial Sequence Synthetic polypeptide
QGHLVESGGDVVLPGGSLRLSCTESGVDLSDFGIHWVRQTPGKGLEWVALIWYDGSKKFYADSVKDRFTISRDNSKNMVYLEMISLRVEDTAMYFCARGIRRGPWFTYWGPGTLVTVST

SEQ ID NO: 2 - protein, 107 aa, Artificial Sequence
  Description of Artificial Sequence Synthetic polypeptide
EIVLTQSPVILSLSPGERATLSCRASQSISSDLAWFQQTPGQAPRLIIYDASNRATGIPARFSGSGSGTDFTLTISSLEPEDFAVYYCQQRSNWPLTFGGGTKVEIK

SEQ ID NO: 3 - protein, 8 aa, Artificial Sequence
  Description of Artificial Sequence Synthetic peptide
GVDLSDFG

SEQ ID NO: 4 - protein, 8 aa, Artificial Sequence
  Description of Artificial Sequence Synthetic peptide
IWYDGSKK

SEQ ID NO: 5 - protein, 12 aa, Artificial Sequence
  Description of Artificial Sequence Synthetic peptide
ARGIRRGPWFTY

SEQ ID NO: 6 - protein, 6 aa, Artificial Sequence
  Description of Artificial Sequence Synthetic peptide
QSISSD

SEQ ID NO: 7 - protein, 3 aa, Artificial Sequence
  Description of Artificial Sequence Synthetic peptide
DAS

SEQ ID NO: 8 - protein, 9 aa, Artificial Sequence
  Description of Artificial Sequence Synthetic peptide
QQRSNWPLT

SEQ ID NO: 9 - protein, 116 aa, Artificial Sequence
  Description of Artificial Sequence Synthetic polypeptide
QLQLVESGGDVVQPGGSLRLSCAASGVVFSDFGLEWVRQAPGKGLEWLAVIWYDGSRKHYADSVKGRFTISRDNSKNMLYLQMNSLRVEDTAMYYCARCHSKEDYWGQGTLVTVSS

SEQ ID NO: 10 - protein, 107 aa, Artificial Sequence
  Description of Artificial Sequence Synthetic polypeptide
EIEVTQSPATLSLSPGERATLSCRASQSIDTDLAWFQQRPGQTPRLIIYDASKRATGIPARFSGGGSGTDFTLTISSLEPEDFAVYYCQQRTTWPLTFGGGTKVEIK

SEQ ID NO: 11 - protein, 8 aa, Artificial Sequence
  Description of Artificial Sequence Synthetic peptide
GVVFSDFG

SEQ ID NO: 12 - protein, 8 aa, Artificial Sequence
  Description of Artificial Sequence Synthetic peptide
IWYDGSRK

SEQ ID NO: 13 - protein, 9 aa, Artificial Sequence
  Description of Artificial Sequence Synthetic peptide
ARCHSKEDY

SEQ ID NO: 14 - protein, 6 aa, Artificial Sequence
  Description of Artificial Sequence Synthetic peptide
QSIDTD

SEQ ID NO: 15 - protein, 3 aa, Artificial Sequence
  Description of Artificial Sequence Synthetic peptide
DAS

SEQ ID NO: 16 - protein, 9 aa, Artificial Sequence
  Description of Artificial Sequence Synthetic peptide
QQRTTWPLT

SEQ ID NO: 17 - protein, 116 aa, Artificial Sequence
  Description of Artificial Sequence Synthetic polypeptide
QVQLVESGGDVVQPGGSLRLSCAASGVAFSDYGMEWVRQAPGKGLEWLAVIWYDGSRKHYADSVKGRFTISRDNSKNILYLQMNSLRAEDTAMYYCARCHSKDDYWGQGTLVTVSS

SEQ ID NO: 18 - protein, 107 aa, Artificial Sequence
  Description of Artificial Sequence Synthetic polypeptide
EIVLTQFPATLSLSPGERATLSCRTSQNIDSDLAWFQQKPGQAPRLIIYDASNRATGIPARFSGGGSGTDFTLTITSLEPEDFAVYYCQQRTTWPLTFGGGTKVEIK

SEQ ID NO: 19 - protein, 8 aa, Artificial Sequence
  Description of Artificial Sequence Synthetic peptide
GVAFSDYG

SEQ ID NO: 20 - protein, 10 aa, Artificial Sequence
  Description of Artificial Sequence Synthetic peptide
VIWYDGSRKH

SEQ ID NO: 21 - protein, 9 aa, Artificial Sequence
  Description of Artificial Sequence Synthetic peptide
ARCHSKDDY

SEQ ID NO: 22 - protein, 6 aa, Artificial Sequence
  Description of Artificial Sequence Synthetic peptide
QNIDSD

SEQ ID NO: 23 - protein, 3 aa, Artificial Sequence
  Description of Artificial Sequence Synthetic peptide
DAS

SEQ ID NO: 24 - protein, 9 aa, Artificial Sequence
  Description of Artificial Sequence Synthetic peptide
QQRTTWPLT

SEQ ID NO: 25 - protein, 116 aa, Artificial Sequence
  Description of Artificial Sequence Synthetic polypeptide
QVQLVESGGDVVQPGGSLRLSCAVSGVTFGDFGFEWVRQAPGKGLEWVAVIWYDGSRKHYAESVRGRFTISRDNSRNMMYLEMTGLRVEDTAKYYCTRSHSHEDYWGQGTLVTVSS

SEQ ID NO: 26 - protein, 107 aa, Artificial Sequence
  Description of Artificial Sequence Synthetic polypeptide
EIVLTQSPATLSLSPGERATLSCRASQSISNYLAWFQQKSGQAPRLIIHDAFKRAAGIPTRFSGSGSGTDFTLTISSLEPEDFAVYYCQQRDNWPLNFGGGTKVEIK

SEQ ID NO: 27 - protein, 8 aa, Artificial Sequence
  Description of Artificial Sequence Synthetic peptide
GVTFGDFG

SEQ ID NO: 28 - protein, 8 aa, Artificial Sequence
  Description of Artificial Sequence Synthetic peptide
IWYDGSRK

SEQ ID NO: 29 - protein, 9 aa, Artificial Sequence
  Description of Artificial Sequence Synthetic peptide
TRSHSHEDY

SEQ ID NO: 30 - protein, 6 aa, Artificial Sequence
  Description of Artificial Sequence Synthetic peptide
QSISNY

SEQ ID NO: 31 - protein, 3 aa, Artificial Sequence
  Description of Artificial Sequence Synthetic peptide
DAF

SEQ ID NO: 32 - protein, 9 aa, Artificial Sequence
  Description of Artificial Sequence Synthetic peptide
QQRDNWPLN

SEQ ID NO: 33 - protein, 119 aa, Artificial Sequence
  Description of Artificial Sequence Synthetic polypeptide
QVQLVESGGDVVQPGGSLRLSCAASGVVFSDYGLYWVRQAPGKGLEWVALIWYDGSKKFYADSVKGRFSISRDNSKNMLYLQMNNLRADDSAIYYCSRGIRQGPWFAYWGQGTRVTVSP

SEQ ID NO: 34 - protein, 107 aa, Artificial Sequence
  Description of Artificial Sequence Synthetic polypeptide
EIVLTQSPATLSLSPGERATLSCRASQSISSDLTWFQQKPGQAPRLIIYDASNRATGIPARFSGSGSGTDFTLTISSLEPEDFVVYYCLQRSDWPLTFGGGTKVEIK

SEQ ID NO: 35 - protein, 8 aa, Artificial Sequence
  Description of Artificial Sequence Synthetic peptide
GVVFSDYG

SEQ ID NO: 36 - protein, 8 aa, Artificial Sequence
  Description of Artificial Sequence Synthetic peptide
IWYDGSKK

SEQ ID NO: 37 - protein, 12 aa, Artificial Sequence
  Description of Artificial Sequence Synthetic peptide
SRGIRQGPWFAY

SEQ ID NO: 38 - protein, 6 aa, Artificial Sequence
  Description of Artificial Sequence Synthetic peptide
QSISSD

SEQ ID NO: 39 - protein, 3 aa, Artificial Sequence
  Description of Artificial Sequence Synthetic peptide
DAS

SEQ ID NO: 40 - protein, 9 aa, Artificial Sequence
  Description of Artificial Sequence Synthetic peptide
LQRSDWPLT

SEQ ID NO: 41 - protein, 116 aa, Artificial Sequence
  Description of Artificial Sequence Synthetic polypeptide
QVQLVESGGDVVQPGGSLRLSCTVSGAVFRDLGMEWVRQAPGKGLEWLAVIWYDGGRKHYADSVKGRFTISRDNSRNMLFLQMNGLRVDDTAMYYCTRSHSTDDYWGQGTLVTVSS

SEQ ID NO: 42 - protein, 107 aa, Artificial Sequence
  Description of Artificial Sequence Synthetic polypeptide
EIVLTQSPATLSVSPGERATLSCRASQSISSDLAWFQQKPGQAPRLIIHGASKRATGIPARFSGSGSGTDFTLTISSLEPEDFAVYYCQQRDSWPLNFGGGTKVEIK

SEQ ID NO: 43 - protein, 8 aa, Artificial Sequence
  Description of Artificial Sequence Synthetic peptide
GAVFRDLG

SEQ ID NO: 44 - protein, 8 aa, Artificial Sequence
  Description of Artificial Sequence Synthetic peptide
IWYDGGRK

SEQ ID NO: 45 - protein, 9 aa, Artificial Sequence
  Description of Artificial Sequence Synthetic peptide
TRSHSTDDY

SEQ ID NO: 46 - protein, 6 aa, Artificial Sequence
  Description of Artificial Sequence Synthetic peptide
QSISSD

SEQ ID NO: 47 - protein, 3 aa, Artificial Sequence
  Description of Artificial Sequence Synthetic peptide
GAS

SEQ ID NO: 48 - protein, 9 aa, Artificial Sequence
  Description of Artificial Sequence Synthetic peptide
QQRDSWPLN

SEQ ID NO: 49 - protein, 117 aa, Artificial Sequence
  Description of Artificial Sequence Synthetic polypeptide
QEWLVESGGDVVQPGGSLRLSCATSKVTFNDFGIHWVRQAPGKGLEWVAIIWYDGSRNHYADSVRGRFTLSRDNSKNMVHLHMSSLRTEDTAMYYCARGIHSNDDYWGQGTMVTVSS

SEQ ID NO: 50 - protein, 107 aa, Artificial Sequence
  Description of Artificial Sequence Synthetic polypeptide
EIVLTQSPATLSLSPGERATLSCRTSQSVSSDLAWFQQKPGQAPRLFIFDASKRVNGIPARFSGSGSGTDFTLTISSLEPEDFAVYYCQQRTDWPLTFGGGSRVEIK

SEQ ID NO: 51 - protein, 8 aa, Artificial Sequence
  Description of Artificial Sequence Synthetic peptide
KVTFNDFG

SEQ ID NO: 52 - protein, 8 aa, Artificial Sequence
  Description of Artificial Sequence Synthetic peptide
IWYDGSRN

SEQ ID NO: 53 - protein, 10 aa, Artificial Sequence
  Description of Artificial Sequence Synthetic peptide
ARGIHSNDDY

SEQ ID NO: 54 - protein, 6 aa, Artificial Sequence
  Description of Artificial Sequence Synthetic peptide
QSVSSD

SEQ ID NO: 55 - protein, 3 aa, Artificial Sequence
  Description of Artificial Sequence Synthetic peptide
DAS

SEQ ID NO: 56 - protein, 9 aa, Artificial Sequence
  Description of Artificial Sequence Synthetic peptide
QQRTDWPLT

SEQ ID NO: 57 - protein, 116 aa, Artificial Sequence
  Description of Artificial Sequence Synthetic polypeptide
QVQLVESGGDVVQSGGSLRLSCAVSGVVFSDYGFEWVRQAPGKGLEWVAVIWYDGSRKHYADSVQGRFTISRDNFRNMLYLQMTGLRVEDTAKYYCTRSHSHEDYWGQGTLVTVSS

SEQ ID NO: 58 - protein, 107 aa, Artificial Sequence
  Description of Artificial Sequence Synthetic polypeptide
EIVLTQSPATLSLSPGERATLSCGASQNIDNSLAWFQQKPGQAPRLIIYDASKRATGIPARFSGSGSGTDFTLTISTLEPEDFAVYYCQQRDHWPLNFGGGTKVEVK

SEQ ID NO: 59 - protein, 8 aa, Artificial Sequence
  Description of Artificial Sequence Synthetic peptide
GVVFSDYG

SEQ ID NO: 60 - protein, 8 aa, Artificial Sequence
  Description of Artificial Sequence Synthetic peptide
IWYDGSRK

SEQ ID NO: 61 - protein, 9 aa, Artificial Sequence
  Description of Artificial Sequence Synthetic peptide
TRSHSHEDY

SEQ ID NO: 62 - protein, 6 aa, Artificial Sequence
  Description of Artificial Sequence Synthetic peptide
QNIDNS

SEQ ID NO: 63 - protein, 3 aa, Artificial Sequence
  Description of Artificial Sequence Synthetic peptide
DAS

SEQ ID NO: 64 - protein, 9 aa, Artificial Sequence
  Description of Artificial Sequence Synthetic peptide
QQRDHWPLN

SEQ ID NO: 65 - protein, 123 aa, Artificial Sequence
  Description of Artificial Sequence Synthetic polypeptide
QVQLVESGGDVVQPGGSLRLSCSASGLVISDYGMNWVRQAPGKGLEWVGLIWYDGSKKYYSDFVKGRFTISRDNSKNILYLQMNNLRAEDTAMYYCARFLIGATRRGNAMDYWGQGTSVTVSS

SEQ ID NO: 66 - protein, 107 aa, Artificial Sequence
  Description of Artificial Sequence Synthetic polypeptide
EVVLTQSPATLSLSPGERATLSCRASQSIDSDLAWSQQKPGQPPRLIIYDASNRATGIPARFSGSGSGTDFTLTISSLEPEDFAVYYCQQRSDWPLTFGGGTKVEIR

SEQ ID NO: 67 - protein, 8 aa, Artificial Sequence
  Description of Artificial Sequence Synthetic peptide
GLVISDYG

SEQ ID NO: 68 - protein, 8 aa, Artificial Sequence
  Description of Artificial Sequence Synthetic peptide
IWYDGSKK

SEQ ID NO: 69 - protein, 16 aa, Artificial Sequence
  Description of Artificial Sequence Synthetic peptide
ARFLIGATRRGNAMDY

SEQ ID NO: 70 - protein, 6 aa, Artificial Sequence
  Description of Artificial Sequence Synthetic peptide
QSIDSD

SEQ ID NO: 71 - protein, 3 aa, Artificial Sequence
  Description of Artificial Sequence Synthetic peptide
DAS

SEQ ID NO: 72 - protein, 9 aa, Artificial Sequence
  Description of Artificial Sequence Synthetic peptide
QQRSDWPLT

SEQ ID NO: 73 - protein, 123 aa, Artificial Sequence
  Description of Artificial Sequence Synthetic polypeptide
QVQLVESGGDVVQPGGSLRLSCSASGLVFRDYGMNWVRQAPGKGLEWVGLIWYDGTKKYYSDFVKGRFTISRDNSKNMLYLQMNNLRAEDTAIYYCARFLIGATRRGNAMDYWGQGTSVIVSS

SEQ ID NO: 74 - protein, 107 aa, Artificial Sequence
  Description of Artificial Sequence Synthetic polypeptide
EVVLTQSPATLSLSPGERATLSCRASQSIDSDLAWSQQKTGQPPRLIIYDASNRATGIPARFSGSGSGTDFTLTISSLEPEDFAVYYCQQRSDWPLTFGGGTKVEIR

SEQ ID NO: 75 - protein, 8 aa, Artificial Sequence
  Description of Artificial Sequence Synthetic peptide
GLVFRDYG

SEQ ID NO: 76 - protein, 8 aa, Artificial Sequence
  Description of Artificial Sequence Synthetic peptide
IWYDGTKK

SEQ ID NO: 77 - protein, 16 aa, Artificial Sequence
  Description of Artificial Sequence Synthetic peptide
ARFLIGATRRGNAMDY

SEQ ID NO: 78 - protein, 6 aa, Artificial Sequence
  Description of Artificial Sequence Synthetic peptide
QSIDSD

SEQ ID NO: 79 - protein, 3 aa, Artificial Sequence
  Description of Artificial Sequence Synthetic peptide
DAS

SEQ ID NO: 80 - protein, 9 aa, Artificial Sequence
  Description of Artificial Sequence Synthetic peptide
QQRSDWPLT

SEQ ID NO: 81 - protein, 113 aa, Artificial Sequence
  Description of Artificial Sequence Synthetic polypeptide
QVQLVESGGDVVQPGGSLRLSCAASGVAFRDYGMHWVRQAPGKGLEWVAVIWYDGSKKYYGDSVKGRFTVSRDNSKNMLYLEMNGLKAEDTAMYYCARNDDYWGQGTLVTVSS

SEQ ID NO: 82 - protein, 107 aa, Artificial Sequence
  Description of Artificial Sequence Synthetic polypeptide
EIVLTQSPATLSLSPGERATLSCRASQSVSNSLSWYQQNPGQSPRLIIYDTSKRATGIPARFSGSGSGTDFTLTINNLETEDFAVYYCHQRSDWPLTFGGGTKVEIK

SEQ ID NO: 83 - protein, 8 aa, Artificial Sequence
  Description of Artificial Sequence Synthetic peptide
GVAFRDYG

SEQ ID NO: 84 - protein, 10 aa, Artificial Sequence
  Description of Artificial Sequence Synthetic peptide
VIWYDGSKKY

SEQ ID NO: 85 - protein, 6 aa, Artificial Sequence
  Description of Artificial Sequence Synthetic peptide
ARNDDY

SEQ ID NO: 86 - protein, 6 aa, Artificial Sequence
  Description of Artificial Sequence Synthetic peptide
QSVSNS

SEQ ID NO: 87 - protein, 3 aa, Artificial Sequence
  Description of Artificial Sequence Synthetic peptide
DTS

SEQ ID NO: 88 - protein, 9 aa, Artificial Sequence
  Description of Artificial Sequence Synthetic peptide
HQRSDWPLT

SEQ ID NO: 89 - protein, 10 aa, Artificial Sequence
  Description of Artificial Sequence Synthetic peptide
LIWYDGSKKF

SEQ ID NO: 90 - protein, 10 aa, Artificial Sequence
  Description of Artificial Sequence Synthetic peptide
VIWYDGSRKH

SEQ ID NO: 91 - protein, 10 aa, Artificial Sequence
  Description of Artificial Sequence Synthetic peptide
VIWYDGSRKH

SEQ ID NO: 92 - protein, 10 aa, Artificial Sequence
  Description of Artificial Sequence Synthetic peptide
LIWYDGSKKF

SEQ ID NO: 93 - protein, 10 aa, Artificial Sequence
  Description of Artificial Sequence Synthetic peptide
VIWYDGGRKH

SEQ ID NO: 94 - protein, 10 aa, Artificial Sequence
  Description of Artificial Sequence Synthetic peptide
IIWYDGSRNH

SEQ ID NO: 95 - protein, 10 aa, Artificial Sequence
  Description of Artificial Sequence Synthetic peptide
VIWYDGSRKH

SEQ ID NO: 96 - protein, 10 aa, Artificial Sequence
  Description of Artificial Sequence Synthetic peptide
LIWYDGSKKY

SEQ ID NO: 97 - protein, 10 aa, Artificial Sequence
  Description of Artificial Sequence Synthetic peptide
LIWYDGTKKY

SEQ ID NO: 98 - protein, 5 aa, Artificial Sequence
  Description of Artificial Sequence Synthetic peptide
GGGGS

SEQ ID NO: 99 - protein, 6 aa, Artificial Sequence
  Description of Artificial Sequence Synthetic peptide
HHHHHH

SEQ ID NO: 100 - protein, 10 aa, Artificial Sequence
  Description of Artificial Sequence Synthetic peptide
LDSPDRPWNP

SEQ ID NO: 101 - DNA, 340 bp, Artificial Sequence
  Description of Artificial Sequence Synthetic polynucleotide
caggtgcagttggtggagtctgggggagacgtggtccagcctggggggtccctgagactctcctgtgcagcgtctggagtcgccttcagggactatggcatgcactgggtccgccaggcaccaggcaaggggctggagtgggtggcagttatatggtatgatggaagtaagaaatattatggagactccgtgaagggccgattcaccgtctccagagacaattccaagaacatgttgtatctggaaatgaacggcctgaaagccgaggacacggcaatgtattattgtgcgaggaacgatgactactggggccagggaaccctggtcaccgtctcctcag

SEQ ID NO: 102 - DNA, 349 bp, Artificial Sequence
  Description of Artificial Sequence Synthetic polynucleotide
caggtgcagctggtggagtctgggggagacgtggtccagccgggggggtccctgagactctcctgtgcggcgtctggagtcgccttcagtgactatggcatggaatgggtccgccaggctccaggcaaggggctggagtggctggcagttatctggtatgatggaagtaggaaacactatgcagactccgtgaagggccgattcaccatctcccgcgacaattccaagaacattctgtatctacaaatgaacagcctgagagccgaggacacggctatgtattactgtgcgagatgccactctaaagatgactactggggccagggaaccctggtcaccgtctcctcag

SEQ ID NO: 103 - DNA, 352 bp, Artificial Sequence
  Description of Artificial Sequence Synthetic polynucleotide
caggagtggttggtggagtcggggggagacgtggtccagccgggggggtccctgagactctcctgtgcgacgtctaaagtcaccttcaatgactttggcattcactgggtccgccaggctccaggcaagggactggagtgggtggcaattatttggtatgatggaagcaggaatcactacgcagactccgtgaggggccgattcaccctctccagggacaattccaaaaacatggtccatcttcacatgagtagcctgagaaccgaggacacggctatgtattattgtgcgcgaggaatacactctaacgatgactattggggccagggaaccatggtcaccgtctcctcag

SEQ ID NO: 104 - DNA, 349 bp, Artificial Sequence
  Description of Artificial Sequence Synthetic polynucleotide
caggttcagctggtggaatctgggggagacgtggtccagccgggggggtccctgagactctcctgtgcagtgtctggagtcaccttcggtgactttggcttcgaatgggtccgccaggctccaggcaagggtctggagtgggtggcagttatttggtacgacggaagcagaaaacattatgcagagtccgtgaggggccgatttaccatctccagagacaattccaggaacatgatgtatctggaaatgactggactgagagtcgaggacacggctaaatattactgtacgagaagtcactctcacgaggactactggggccagggaaccctggtcaccgtctcctcag

SEQ ID NO: 105 - DNA, 349 bp, Artificial Sequence
  Description of Artificial Sequence Synthetic polynucleotide
caggtgcagctggtggagtctgggggagacgtggtccagcctggggggtccctgagactctcctgtacagtgtctggagccgtcttccgtgacttgggcatggaatgggtccgtcaggccccaggcaaggggctggaatggttggcagttatatggtacgacggaggtagaaaacactatgcggactccgtgaagggccggttcaccatctccagagacaattccaggaacatgctctttctgcaaatgaatggactgagagtcgacgacacggctatgtattactgtacgagaagccactctaccgatgattactggggccagggaaccctggtcaccgtctcctcag

SEQ ID NO: 106 - DNA, 349 bp, Artificial Sequence
  Description of Artificial Sequence Synthetic polynucleotide
caggtgcagttggtggagtctgggggagacgtggtccagtcgggggggtccctgagactctcctgtgcagtgtctggagtcgtcttcagtgattatggcttcgaatgggtccgccaggctccaggcaaggggctggagtgggtggcagttatatggtacgacggaagtaggaaacattatgcagactccgtccagggccgattcaccatttccagagacaatttccggaacatgttgtatctacaaatgactggactgagagtcgaggacacggctaaatactattgtacgagaagccactctcacgaggactactggggccagggaaccctggtcaccgtctcctcag

SEQ ID NO: 107 - DNA, 349 bp, Artificial Sequence
  Description of Artificial Sequence Synthetic polynucleotide
caattacaactggtggagtctgggggagacgtggtccagcctggggggtccctgagactctcctgtgcggcgtctggggtcgtcttcagtgactttggcctggaatgggtccgccaggctccaggcaaggggctggagtggctggcagttatctggtatgatggaagtcggaaacattatgcagactccgtgaagggccgattcaccatctccagggacaattccaagaacatgctctatctgcaaatgaacagtctgagagtcgaggacacggctatgtactactgtgcgcgatgtcactctaaagaggactactggggccagggaacccttgtcaccgtctcctcag

SEQ ID NO: 108 - DNA, 370 bp, Artificial Sequence
  Description of Artificial Sequence Synthetic polynucleotide
caggtgcagctggtggagtctgggggagacgtggtccagcctggggggtccctgagactctcctgttcagcgtctggactcgtattcagagactatggcatgaactgggtccgccaggctccaggcaaggggctggagtgggtcggacttatatggtatgatggaactaaaaaatattattcagacttcgtgaagggccgattcaccatctccagagacaattccaagaacatgttgtatctacaaatgaacaacctgagagccgaggacacggctatttattactgtgcgagatttctaataggtgcgacgaggaggggcaatgctatggactactggggtcaagggacctcagtcatcgtctcatcag

SEQ ID NO: 109 - DNA, 370 bp, Artificial Sequence
  Description of Artificial Sequence Synthetic polynucleotide
caggtgcagttggtggagtctgggggagacgtggtccagcctggggggtccctgagactctcctgttcagcgtctgggctcgtaatcagtgactatggcatgaactgggtccgccaggctccaggcaaggggctggagtgggtcggacttatatggtatgatggaagtaaaaaatattactcagacttcgtgaagggccgattcaccatctccagagacaattccaagaacatattgtatctacaaatgaacaacctgagagccgaggacacggctatgtattactgtgcgagatttctaataggtgcgacgaggaggggcaatgctatggactattggggtcaaggaacctcagtcaccgtctcatcag

SEQ ID NO: 110 - DNA, 358 bp, Artificial Sequence
  Description of Artificial Sequence Synthetic polynucleotide
caggggcacctggtggagtctgggggagacgtggtcctgcctggggggtccctgagactctcctgtacagagtctggtgtcgacctcagtgactttggcatacattgggtccgccagactccaggcaagggtctggagtgggtggcacttatctggtatgatggaagtaaaaaattttatgctgactccgtgaaggaccgattcaccatttccagagacaattccaagaatatggtgtatctggaaatgatcagcctgagagtcgaggatacggctatgtacttctgtgcgagagggatacgacgggggccctggttcacttactggggcccagggactctggttacagtctctacag

SEQ ID NO: 111 - DNA, 370 bp, Artificial Sequence
  Description of Artificial Sequence Synthetic polynucleotide
caggtgcagttggtggagtctgggggagacgtggtccagccgggggggtccctgagactctcctgtgcagcgtctggagtcgccttcaggaactatggcatgcactgggtccgccaggctccaggcaagggcctggagtgggtggcaattatatggtatgatggaagtaataaatattatgcagactccgtgaagggccgcttcaccatctccagagacaattccaagaatatgttgtatcttcaaatgaatagcctgagagccgaggacacggctatgtattactgtgcgagactctctataggtacgacccattactttgatacggacgactactggggtcaaggaacctcagtcaccgtctcctcag

SEQ ID NO: 112 - DNA, 358 bp, Artificial Sequence
  Description of Artificial Sequence Synthetic polynucleotide
caggtgcaactggtggagtctgggggagacgtggtccagccgggggggtccctgagactctcctgtgcagcgtctggagtcgtcttcagtgactatggcttgtattgggtccgccaggctccaggcaagggcctggagtgggtggcccttatatggtatgatgggagtaagaaattttatgctgactccgtgaagggccgattctccatctccagagacaattccaagaacatgttgtatttacaaatgaataatttgagagccgacgactcggctatttattattgttcgagagggatacgacaggggccatggtttgcttactggggccaagggactcgtgtcactgtctctccag

SEQ ID NO: 113 - DNA, 322 bp, Artificial Sequence
  Description of Artificial Sequence Synthetic polynucleotide
gaaattgtgttgacacagtctccagccaccctgtctttgtctccaggggaaagagccaccctctcctgcagggccagtcagagtgttagcaactccttatcctggtaccaacagaaccctggccagtctcccaggctcatcatctatgatacatccaagagggccactggcatcccagccaggttcagtggcagtggatctgggacagacttcactctcaccatcaacaatctagagactgaagattttgcagtttattactgtcaccagcgtagcgactggcctctcactttcggcggagggaccaaggtggagatcaaac

SEQ ID NO: 114 - DNA, 322 bp, Artificial Sequence
  Description of Artificial Sequence Synthetic polynucleotide
gaaattgtgttgacacagtttccggccaccctgtctctgtctcccggggaaagagccaccctctcctgcaggaccagtcagaatattgacagcgacttagcctggttccaacagaaacctggccaggctcccaggctcatcatctatgatgcatccaacagggccactggcatcccagccaggttcagtggcggtgggtctgggacagacttcactctcaccatcaccagcctagagcctgaagattttgcagtttattactgtcagcagcgtaccacctggcctctcactttcggcggagggaccaaggtggagatcaaac

SEQ ID NO: 115 - DNA, 322 bp, Artificial Sequence
  Description of Artificial Sequence Synthetic polynucleotide
gaaattgtgttgacacagtctccagccaccctgtctttgtctccaggggaaagagccaccctctcctgccggaccagtcagagtgttagcagcgacttagcctggttccaacagaaacctggccaggctcccaggctcttcatatttgatgcatccaaaagggtcaatggcatcccagccaggttcagtggcagtgggtctgggacagacttcactctcaccatcagcagcctggaacctgaagattttgcagtttattattgtcagcaacgtaccgactggcctctcactttcggcggagggtccagggtggagatcaaac

SEQ ID NO: 116 - DNA, 322 bp, Artificial Sequence
  Description of Artificial Sequence Synthetic polynucleotide
gaaattgtgttgacacagtctccagccaccctgtctttgtctccaggggaaagagccaccctctcctgtagggccagtcagagtattagcaactacttagcctggttccaacagaaatctggccaggctcccaggctcatcatccatgatgcatttaaacgggccgctggcatcccaaccaggttcagtggcagtgggtctgggacagacttcactctcaccatcagcagtctagagcctgaagattttgcagtttattattgtcagcagcgtgacaactggcctctcaatttcggcggagggactaaggtggagatcaaac

SEQ ID NO: 117 - DNA, 322 bp, Artificial Sequence
  Description of Artificial Sequence Synthetic polynucleotide
gaaattgtgttgacacagtcgccagccactctgtctgtgtctccaggggaaagagccaccctctcctgtagggccagtcagagtattagcagcgacttagcctggttccaacagaaacctggccaggctcccaggctcatcatccatggtgcatccaaaagggccactggcatcccagccaggttcagtggcagtgggtctgggacagacttcactctcaccatcagcagcctagagcctgaagattttgcggtttattactgtcagcagcgtgacagctggcctctcaatttcggcggagggaccaaggtggagatcaaac

SEQ ID NO: 118 - DNA, 322 bp, Artificial Sequence
  Description of Artificial Sequence Synthetic polynucleotide
gaaattgtgttgacacagtctccagccaccctgtctttgtctccaggggaaagagccaccctctcctgtggggccagtcagaatattgacaactccttagcctggttccaacagaaacctggccaggctcccaggctcatcatctatgatgcatctaaaagggccactggcatcccagccaggttcagtggcagtgggtctgggacagacttcactctcaccatcagcactctagagcctgaagattttgcagtttattattgtcagcagcgtgaccattggcctctcaatttcggcggagggaccaaggtggaagtcaaac

SEQ ID NO: 119 - DNA, 322 bp, Artificial Sequence
  Description of Artificial Sequence Synthetic polynucleotide
gaaattgaagtgacacagtctccggccaccctgtccttgtctccaggggaaagagccaccctctcctgtagggccagtcagagtattgacaccgacttagcctggttccagcagagacctggccagactcccagactcatcatctatgatgcatccaaaagggccactggcatcccagccaggttcagtggcggtgggtctgggacagacttcactctcaccatcagcagcctagagcctgaagattttgcagtttactactgtcagcagcgtaccacctggcctctcactttcggcggagggaccaaggtggagatcaaac

SEQ ID NO: 120 - DNA, 322 bp, Artificial Sequence
  Description of Artificial Sequence Synthetic polynucleotide
gaagttgtgttgacacagtctccagccaccctgtctttgtctccaggggaaagagccaccctctcctgcagggccagtcagagtattgacagcgacttagcctggtcccaacagaaaactggccagcctcccagactcatcatctatgatgcatccaacagggccactggcatcccagccaggttcagtggcagtgggtctgggacagacttcactctcaccatcagtagtctagagcctgaagattttgcagtttattattgtcagcaacgtagcgactggcctctcactttcggcggagggaccaaggtggagatcagac

SEQ ID NO: 121 - DNA, 322 bp, Artificial Sequence
  Description of Artificial Sequence Synthetic polynucleotide
gaagttgtgttgacacagtctccagccaccctgtctttgtctccaggggaaagagccaccctctcctgcagggccagtcagagtattgacagcgacttagcctggtcccaacagaaacctggccagcctcccagactcatcatctatgatgcatccaacagggccactggcatcccagccaggttcagtggcagtgggtctgggacagacttcactctcaccatcagcagcctagagcctgaagattttgcagtttattattgtcagcaacgtagcgactggcctctcactttcggcggagggaccaaggtggagatcagac

SEQ ID NO: 122 - DNA, 322 bp, Artificial Sequence
  Description of Artificial Sequence Synthetic polynucleotide
gaaattgtgttgacacagtctccagtcatcctgtctttgtctccaggggaaagagccaccctctcctgcagggccagtcagagtattagcagcgacttggcctggttccaacagacacctggccaggctcccaggctcatcatctatgatgcatccaacagggccactggcatcccagccaggttcagtggcagtgggtctgggacagacttcactctcaccatcagcagcctagagcctgaagattttgcagtttattactgtcagcagcggagcaactggcctctcactttcggcggagggaccaaggtggagatcaaac

SEQ ID NO: 123 - DNA, 322 bp, Artificial Sequence
  Description of Artificial Sequence Synthetic polynucleotide
gaaattgtgttgacacagtctccagccaccctgtctttgtctccaggggaaagagccaccctctcctgcagggccagtcagagtgttagcagctacttagcctggtaccaacagaaggttggccaggctcccaggctcatcatctttgatgcatccaacagggccactggcatcccagccaggttcagtggcagtgggtctgggacagacttcactctcaccatcaccagcctagatcctgaagattttgcagtttattactgtcagcaacgtagcgcctggcctctcactttcggcggagggaccaaggtggagatcagac

SEQ ID NO: 124 - DNA, 322 bp, Artificial Sequence
  Description of Artificial Sequence Synthetic polynucleotide
gaaattgtgctgacacagtctccagccaccctgtctttgtctccaggggaaagagccaccctctcctgcagggccagtcagagcattagcagcgacttaacctggttccaacagaaacctggccaggctcccaggctcatcatctatgatgcatccaacagggccactggcatcccagccaggttcagtggcagtgggtctgggacagacttcactctcaccatcagtagcctcgagcctgaagattttgtagtttattactgtctgcaacgtagcgactggcctctcactttcggcggagggaccaaggtggagatcaaac

SEQ ID NO: 125 - protein, 113 aa, Artificial Sequence
  Description of Artificial Sequence Synthetic polypeptide
QVQLVESGGDVVQPGGSLRLSCAASGVAFSNYGMHWVRQAPGKGLEWVAVIWYDGSNKYYADSVKGRFTISRDNSKNMLYLQMNSLRAEDTAMYYCARNDDYWGQGTLVTVSS

SEQ ID NO: 126 - protein, 113 aa, Artificial Sequence [sequence omitted]

SEQ ID NO: 127 - protein, 107 aa, Artificial Sequence
  Description of Artificial Sequence Synthetic polypeptide
EIVLTQSPATLSLSPGERATLSCRASQSVSSYLAWYQQKPGQAPRLIIYDASNRATGIPARFSGSGSGTDFTLTISSLEPEDFAVYYCQQRSNWPLTFGGGTKVEIK

SEQ ID NO: 128 - protein, 107 aa, Artificial Sequence [sequence omitted]

SEQ ID NO: 129 - DNA, 340 bp, Artificial Sequence
  Description of Artificial Sequence Synthetic polynucleotide
caggtgcagctggtggagtctgggggagacgtggtccagcctggggggtccctgagactctcctgtgcagcgtctggagtcgccttcagtaactatggcatgcactgggtccgccaggctccaggcaaggggctggagtgggtggcagttatatggtatgatggaagtaataaatactatgcagactccgtgaagggccgattcaccatctccagagacaattccaagaacatgctgtatctgcaaatgaacagcctgagagccgaggacacggctatgtattactgtgcgaggaacgatgactactggggccagggaaccctggtcaccgtctcctcag

SEQ ID NO: 130 - DNA, 322 bp, Artificial Sequence
  Description of Artificial Sequence Synthetic polynucleotide
gaaattgtgttgacacagtctccagccaccctgtctttgtctccaggggaaagagccaccctctcctgcagggccagtcagagtgttagcagctacttagcctggtaccaacagaaacctggccaggctcccaggctcatcatctatgatgcatccaacagggccactggcatcccagccaggttcagtggcagtgggtctgggacagacttcactctcaccatcagcagcctagagcctgaagattttgcagtttattactgtcagcagcgtagcaactggcctctcactttcggcggagggaccaaggtggagatcaaac

SEQ ID NO: 131 - protein, 123 aa, Artificial Sequence
  Description of Artificial Sequence Synthetic polypeptide
QVQLVESGGDVVQPGGSLRLSCAASGVAFRNYGMHWVRQAPGKGLEWVAIIWYDGSNKYYADSVKGRFTISRDNSKNMLYLQMNSLRAEDTAMYYCARLSIGTTHYFDTDDYWGQGTSVTVSS

SEQ ID NO: 132 - protein, 107 aa, Artificial Sequence
  Description of Artificial Sequence Synthetic polypeptide
EIVLTQSPATLSLSPGERATLSCRASQSVSSYLAWYQQKVGQAPRLIIFDASNRATGIPARFSGSGSGTDFTLTITSLDPEDFAVYYCQQRSAWPLTFGGGTKVEIR

(19) United States
(12) Patent Application Publication
(10) Pub. No.: US 2024/0294641 A1
(43) Pub. Date: Sep. 5, 2024

(54) NOVEL ANTI-PD1 ANTIBODIES FOR INHIBITING T-CELL ACTIVITY

(71) Applicant: THE CHILDREN'S MEDICAL CENTER CORPORATION, Boston, MA (US)

(72) Inventors: Frederick W. ALT, Cambridge, MA (US); Ming TIAN, Boston, MA (US); Hwei-Ling CHENG, Northborough, MA (US)

(73) Assignee: THE CHILDREN'S MEDICAL CENTER CORPORATION, Boston, MA (US)

(21) Appl. No.: 18/005,286

(22) PCT Filed: Jul. 13, 2021
(86) PCT No.: PCT/US2021/041407

§ 371 Date: Jan. 12, 2023

Related U.S. Application Data
(60) Provisional application No. 63/124,270, filed on Dec. 11, 2020.
(60) Provisional application No. 62/705,726, filed on Jul. 13, 2020.

(51) Int. Cl. C07K 16/28; A61K 47/68

(52) U.S. Cl. CPC C07K 16/2818; A61K 47/6849; C07K 2317/21; C07K 2317/24; C07K 2317/565

(57) ABSTRACT

The technology described herein is directed to novel anti-PD1 antibody reagents (e.g., antibodies, antigen-binding fragments thereof, and/or chimeric antigen receptors). Also described herein are antibody-drug conjugates or kits comprising the disclosed antibody reagents, as well as methods of treating an autoimmune or auto-inflammatory disorder by administering the disclosed antibody reagents.

### CROSS-REFERENCE TO RELATED APPLICATIONS

This application is a 35 U.S.C. § 371 National Phase Entry Application of International Application No. PCT/US2020/041407 filed Jul. 13, 2021, which designates the U.S. and claims benefit under 35 U.S.C. § 119(e) of U.S. Provisional Application No. 62/705,726 filed Jul. 13, 2020, and U.S. Provisional Application No. 63/124,270 filed Dec. 11, 2020, the contents of which are incorporated herein by reference in their entireties.

### SEQUENCE LISTING

The instant application contains a Sequence Listing which has been submitted electronically in ASCII file format and is hereby incorporated by reference in its entirety. Said ASCII copy, created on Jun. 15, 2023, is named 701039-097810USPX_SL.txt and is 64,874 bytes in size.

### TECHNICAL FIELD

The technology described herein relates to compositions, systems, and methods directed at anti-PD1 therapeutics.

### BACKGROUND

PD1 (Programmed Death 1) is an immune checkpoint protein known to play a critical role in regulation of immune responses. PD1 serves as a negative regulator of immune system activity. Inhibition of PD1 is a known immunotherapy approach for the treatment of cancer. By inhibiting PD1, the patient's immune system is able to more effectively target the cancer. As antibodies specific for a given target are typically inhibitory of that target's activity, antibodies are often used to target PD1 in such anti-cancer therapies. However, there exists a need for developing anti-PD1 antibodies that inhibit T-cell responses, thereby permitting treatment of disease in which the immune system is overactive. The present disclosure is directed to solving this problem and addressing other needs.

### SUMMARY

Described herein are the development and characterization of anti-PD1 antibodies demonstrated to have high specificity and binding affinity. These anti-PD1 antibodies can, surprisingly, inhibit, rather than activate, T-cell responses, by agonizing or stimulating PD1. Such stimulating or agonizing antibodies for PD1 are atypical and surprising, as anti-PD1 antibodies are usually inhibitory for PD1 function when bound to PD1. These antibodies are developed using a system which harnesses the natural affinity maturation processes in order to provide new therapeutic antibodies with significantly improved affinity and specificity.

In one aspect of any of the embodiments, described herein is an antibody, antibody reagent, antigen-binding fragment thereof, or chimeric antigen receptor (CAR), that specifically binds an PD1 polypeptide, said antibody reagent, antigen-binding portion thereof, or CAR comprising at least one heavy or light chain complementarity determining region (CDR) selected from the group consisting of:
(a) a heavy chain CDR1 having the amino acid sequence of SEQ ID NO: 3;
(b) a heavy chain CDR2 having the amino acid sequence of SEQ ID NO: 4;
(c) a heavy chain CDR3 having the amino acid sequence of SEQ ID NO: 5;
(d) a light chain CDR1 having the amino acid sequence of SEQ ID NO: 6;
(e) a light chain CDR2 having the amino acid sequence of SEQ ID NO: 7; and
(f) a light chain CDR3 having the amino acid sequence of SEQ ID NO: 8, or
   selected from the group consisting of:
(a) a heavy chain CDR1 having the amino acid sequence of SEQ ID NO: 19;
(b) a heavy chain CDR2 having the amino acid sequence of SEQ ID NO: 20;
(c) a heavy chain CDR3 having the amino acid sequence of SEQ ID NO: 21;
(d) a light chain CDR1 having the amino acid sequence of SEQ ID NO: 22;
(e) a light chain CDR2 having the amino acid sequence of SEQ ID NO: 23; and
(f) a light chain CDR3 having the amino acid sequence of SEQ ID NO: 24; or
   a conservative substitution variant of one or more of (a)-(f).

In some embodiments of any of the aspects, the antibody, antibody reagent, antigen-binding portion thereof, or CAR includes heavy chain CDRs having the amino acid sequences of SEQ ID NOs: 3-5, or 19-21, or a conservative substitution variant of such amino acid sequence. In some embodiments of any of the aspects, the antibody, antibody reagent, antigen-binding portion thereof, or CAR includes light chain CDRs having the amino acid sequences of SEQ ID NOs: 6-8, or 22-24, or a conservative substitution variant of such amino acid sequence.

In some embodiments of any of the aspects, the antibody, antibody reagent, antigen-binding portion thereof, or CAR includes: heavy chain CDRs having the amino acid sequences of SEQ ID NOs: 3-5 and light chain CDRs having the amino acid sequences of SEQ ID NOs: 6-8 or a conservative substitution variant of such amino acid sequence; or heavy chain CDRs having the amino acid sequences of SEQ ID NOs: 19-21 and light chain CDRs having the amino acid sequences of SEQ ID NOs: 22-24 or a conservative substitution variant of such amino acid sequence.

In one aspect of any of the embodiments, described herein is a first antibody, antibody reagent, antigen-binding portion thereof, or CAR that specifically binds an PD1 polypeptide, and can compete for binding of PD1 with a second antibody comprising: heavy chain CDRs having the amino acid sequences of SEQ ID NOs: 3-5 and light chain CDRs having the amino acid sequences of SEQ ID NOs: 6-8 or a conservative substitution variant of such amino acid sequence; or heavy chain CDRs having the amino acid sequences of SEQ ID NOs: 19-21 and light chain CDRs having the amino acid sequences of SEQ ID NOs: 22-24 or a conservative substitution variant of such amino acid sequence.

In some embodiments of any of the aspects, the first antibody, antibody reagent, antigen-binding fragment thereof, or chimeric antigen receptor (CAR) includes at least one heavy or light chain complementarity determining region (CDR) selected from the group consisting of:
(a) a heavy chain CDR1 having the amino acid sequence of SEQ ID NO: 3;
(b) a heavy chain CDR2 having the amino acid sequence of SEQ ID NO: 4;
(c) a heavy chain CDR3 having the amino acid sequence of SEQ ID NO: 5;
(d) a light chain CDR1 having the amino acid sequence of SEQ ID NO: 6;
(e) a light chain CDR2 having the amino acid sequence of SEQ ID NO: 7; and
(f) a light chain CDR3 having the amino acid sequence of SEQ ID NO: 8; or
   selected from the group consisting of:
(a) a heavy chain CDR1 having the amino acid sequence of SEQ ID NO: 19;
(b) a heavy chain CDR2 having the amino acid sequence of SEQ ID NO: 20;
(c) a heavy chain CDR3 having the amino acid sequence of SEQ ID NO: 21;
(d) a light chain CDR1 having the amino acid sequence of SEQ ID NO: 22;
(e) a light chain CDR2 having the amino acid sequence of SEQ ID NO: 23; and
(f) a light chain CDR3 having the amino acid sequence of SEQ ID NO: 24; or
   a conservative substitution variant of one or more of (a)-(f).

In some embodiments of any of the aspects, the antibody, antibody reagent, antigen-binding portion thereof, or CAR includes the heavy chain variable region sequence of SEQ ID NO: 1 or 17. In some embodiments of any of the aspects, the antibody, antibody reagent, antigen-binding portion thereof, or CAR includes the light chain variable region sequence of SEQ ID NO: 2 or 18. In some embodiments of any of the aspects, the antibody, antibody reagent, antigen-binding portion thereof, or CAR includes: the heavy chain variable region sequence of SEQ ID NO: 1 and the light chain variable region sequence of SEQ ID NO: 2; or the heavy chain variable region sequence of SEQ ID NO: 17 and the light chain variable region sequence of SEQ ID NO: 18. In some embodiments of any of the aspects, the antibody, antibody reagent, antigen-binding portion thereof, or CAR further includes a conservative substitution in a sequence not comprised by a CDR.

In some embodiments of any of the aspects, the antibody reagent or antigen-binding fragment thereof is fully human or fully humanized. In some embodiments of any of the aspects, the antibody reagent or antigen-binding fragment thereof is fully humanized except for the CDR sequences. In some embodiments of any of the aspects, the antibody reagent or antigen-binding fragment is selected from the group consisting of: an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, and a bispecific antibody.

In some embodiments of any of the aspects, described herein is a composition, kit, or combination comprising: (i) the antibody, antibody reagent, antigen-binding portion thereof, or CAR as described herein, or an antibody, antibody reagent, antigen-binding portion thereof, or CAR comprising at least one heavy or light chain complementarity determining region (CDR) selected from the group consisting of:
(a) a heavy chain CDR1 having the amino acid sequence of SEQ ID NO: 11;
(b) a heavy chain CDR2 having the amino acid sequence of SEQ ID NO: 12;
(c) a heavy chain CDR3 having the amino acid sequence of SEQ ID NO: 13;
(d) a light chain CDR1 having the amino acid sequence of SEQ ID NO: 14;
(e) a light chain CDR2 having the amino acid sequence of SEQ ID NO: 15; and
(f) a light chain CDR3 having the amino acid sequence of SEQ ID NO: 16, or
   selected from the group consisting of:
(a) a heavy chain CDR1 having the amino acid sequence of SEQ ID NO: 27;
(b) a heavy chain CDR2 having the amino acid sequence of SEQ ID NO: 28;
(c) a heavy chain CDR3 having the amino acid sequence of SEQ ID NO: 29;
(d) a light chain CDR1 having the amino acid sequence of SEQ ID NO: 30;
(e) a light chain CDR2 having the amino acid sequence of SEQ ID NO: 31; and
(f) a light chain CDR3 having the amino acid sequence of SEQ ID NO: 32, or
   selected from the group consisting of:
(a) a heavy chain CDR1 having the amino acid sequence of SEQ ID NO: 35;
(b) a heavy chain CDR2 having the amino acid sequence of SEQ ID NO: 36;
(c) a heavy chain CDR3 having the amino acid sequence of SEQ ID NO: 37;
(d) a light chain CDR1 having the amino acid sequence of SEQ ID NO: 38;
(e) a light chain CDR2 having the amino acid sequence of SEQ ID NO: 39; and
(f) a light chain CDR3 having the amino acid sequence of SEQ ID NO: 40, or
   selected from the group consisting of:
(a) a heavy chain CDR1 having the amino acid sequence of SEQ ID NO: 43;
(b) a heavy chain CDR2 having the amino acid sequence of SEQ ID NO: 44;
(c) a heavy chain CDR3 having the amino acid sequence of SEQ ID NO: 45;
(d) a light chain CDR1 having the amino acid sequence of SEQ ID NO: 46;
(e) a light chain CDR2 having the amino acid sequence of SEQ ID NO: 47; and
(f) a light chain CDR3 having the amino acid sequence of SEQ ID NO: 48, or
   selected from the group consisting of:
(a) a heavy chain CDR1 having the amino acid sequence of SEQ ID NO: 51;
(b) a heavy chain CDR2 having the amino acid sequence of SEQ ID NO: 52;
(c) a heavy chain CDR3 having the amino acid sequence of SEQ ID NO: 53;
(d) a light chain CDR1 having the amino acid sequence of SEQ ID NO: 54;
(e) a light chain CDR2 having the amino acid sequence of SEQ ID NO: 55; and
(f) a light chain CDR3 having the amino acid sequence of SEQ ID NO: 56, or
   selected from the group consisting of:
(a) a heavy chain CDR1 having the amino acid sequence of SEQ ID NO: 59;
(b) a heavy chain CDR2 having the amino acid sequence of SEQ ID NO: 60;
(c) a heavy chain CDR3 having the amino acid sequence of SEQ ID NO: 61;
(d) a light chain CDR1 having the amino acid sequence of SEQ ID NO: 62;
(e) a light chain CDR2 having the amino acid sequence of SEQ ID NO: 63; and
(f) a light chain CDR3 having the amino acid sequence of SEQ ID NO: 64, or
   selected from the group consisting of:
(a) a heavy chain CDR1 having the amino acid sequence of SEQ ID NO: 67;
(b) a heavy chain CDR2 having the amino acid sequence of SEQ ID NO: 68;
(c) a heavy chain CDR3 having the amino acid sequence of SEQ ID NO: 69;
(d) a light chain CDR1 having the amino acid sequence of SEQ ID NO: 70;
(e) a light chain CDR2 having the amino acid sequence of SEQ ID NO: 71; and
(f) a light chain CDR3 having the amino acid sequence of SEQ ID NO: 72, or
   selected from the group consisting of:
(a) a heavy chain CDR1 having the amino acid sequence of SEQ ID NO: 75;
(b) a heavy chain CDR2 having the amino acid sequence of SEQ ID NO: 76;
(c) a heavy chain CDR3 having the amino acid sequence of SEQ ID NO: 77;
(d) a light chain CDR1 having the amino acid sequence of SEQ ID NO: 78;
(e) a light chain CDR2 having the amino acid sequence of SEQ ID NO: 79; and
(f) a light chain CDR3 having the amino acid sequence of SEQ ID NO: 80, or
a conservative substitution variant of one or more of (a)-(f), or
an antibody, antibody reagent, or antigen-binding portion thereof comprising the heavy chain variable region sequence of SEQ ID NO: 9, or
an antibody, antibody reagent, or antigen-binding portion thereof comprising the light chain variable region sequence of SEQ ID NO: 10, or
an antibody, antibody reagent, or antigen-binding portion thereof comprising the heavy chain variable region sequence of SEQ ID NO: 25, or
   an antibody, antibody reagent, or antigen-binding portion thereof comprising the light chain variable region sequence of SEQ ID NO: 26, or
an antibody, antibody reagent, or antigen-binding portion thereof comprising the heavy chain variable region sequence of SEQ ID NO: 33, or
an antibody, antibody reagent, or antigen-binding portion thereof comprising the light chain variable region sequence of SEQ ID NO: 34, or
an antibody, antibody reagent, or antigen-binding portion thereof comprising the heavy chain variable region sequence of SEQ ID NO: 41, or
an antibody, antibody reagent, or antigen-binding portion thereof comprising the light chain variable region sequence of SEQ ID NO: 42, or
an antibody, antibody reagent, or antigen-binding portion thereof comprising the heavy chain variable region sequence of SEQ ID NO: 49, or
an antibody, antibody reagent, or antigen-binding portion thereof comprising the light chain variable region sequence of SEQ ID NO: 50, or
an antibody, antibody reagent, or antigen-binding portion thereof comprising the heavy chain variable region sequence of SEQ ID NO: 57, or
an antibody, antibody reagent, or antigen-binding portion thereof comprising the light chain variable region sequence of SEQ ID NO: 58, or
an antibody, antibody reagent, or antigen-binding portion thereof comprising the heavy chain variable region sequence of SEQ ID NO: 65, or
an antibody, antibody reagent, or antigen-binding portion thereof comprising the light chain variable region sequence of SEQ ID NO: 66, or
an antibody, antibody reagent, or antigen-binding portion thereof comprising the heavy chain variable region sequence of SEQ ID NO: 73, or
an antibody, antibody reagent, or antigen-binding portion thereof comprising the light chain variable region sequence of SEQ ID NO: 74; and
(ii) an immunosuppressive agent.

In some embodiments of any of the aspects, the antibody, antibody reagent, or antigen-binding portion thereof is conjugated to the immunosuppressive agent.

In some embodiments of any of the aspects, described herein is a nucleic acid sequence encoding the antibody, antibody reagent, antigen-binding fragment thereof, or CAR as described herein. In some embodiments of any of the aspects, described herein is a cell comprising the antibody, antibody reagent, antigen-binding fragment thereof, or CAR as described herein or the nucleic acid sequence as described herein. In some embodiments of any of the aspects, described herein is a pharmaceutical composition comprising the antibody, antibody reagent, antigen-binding fragment thereof, or CAR as described herein; or the composition, kit, or combination as described herein; or the cell as described herein, and a pharmaceutically acceptable carrier.

In some embodiments of any of the aspects, described herein is a solid support comprising the antibody, antibody reagent, antigen-binding fragment thereof, or CAR as described herein. In some embodiments of any of the aspects, the antibody, antibody reagent or antigen-binding fragment thereof is detectably labeled. In some embodiments of any of the aspects, the solid support comprises a particle, a bead, a polymer, or a substrate.

In some embodiments of any of the aspects, described herein is a kit for the detection of PD1 polypeptide in a sample. The kit includes at least a first antibody, antibody reagent, antigen-binding fragment thereof, or CAR as described herein and immobilized on a solid support and comprising a detectable label.

In some embodiments of any of the aspects, described herein is a molecular complex comprising at least one antibody, antibody reagent, antigen-binding fragment thereof, or CAR as described herein bound to an PD1 polypeptide.

In one aspect of any of the embodiments, described herein is a method of treating an autoimmune disorder or an auto-inflammatory disorder in a subject in need thereof, the method includes administering (i) the antibody, antibody reagent, antigen-binding portion thereof, or CAR as described herein or an antibody, antibody reagent, antigen-binding portion thereof, or CAR comprising at least one heavy or light chain complementarity determining region (CDR) selected from the group consisting of:
(a) a heavy chain CDR1 having the amino acid sequence of SEQ ID NO: 11;
(b) a heavy chain CDR2 having the amino acid sequence of SEQ ID NO: 12;
(c) a heavy chain CDR3 having the amino acid sequence of SEQ ID NO: 13;
(d) a light chain CDR1 having the amino acid sequence of SEQ ID NO: 14;
(e) a light chain CDR2 having the amino acid sequence of SEQ ID NO: 15; and
(f) a light chain CDR3 having the amino acid sequence of SEQ ID NO: 16, or
selected from the group consisting of:
(a) a heavy chain CDR1 having the amino acid sequence of SEQ ID NO: 27;
(b) a heavy chain CDR2 having the amino acid sequence of SEQ ID NO: 28;
(c) a heavy chain CDR3 having the amino acid sequence of SEQ ID NO: 29;
(d) a light chain CDR1 having the amino acid sequence of SEQ ID NO: 30;
(e) a light chain CDR2 having the amino acid sequence of SEQ ID NO: 31; and
(f) a light chain CDR3 having the amino acid sequence of SEQ ID NO: 32, or
   selected from the group consisting of:
(a) a heavy chain CDR1 having the amino acid sequence of SEQ ID NO: 35;
(b) a heavy chain CDR2 having the amino acid sequence of SEQ ID NO: 36;
(c) a heavy chain CDR3 having the amino acid sequence of SEQ ID NO: 37;
(d) a light chain CDR1 having the amino acid sequence of SEQ ID NO: 38;
(e) a light chain CDR2 having the amino acid sequence of SEQ ID NO: 39; and
(f) a light chain CDR3 having the amino acid sequence of SEQ ID NO: 40, or
   selected from the group consisting of:
(a) a heavy chain CDR1 having the amino acid sequence of SEQ ID NO: 43;
(b) a heavy chain CDR2 having the amino acid sequence of SEQ ID NO: 44;
(c) a heavy chain CDR3 having the amino acid sequence of SEQ ID NO: 45;
(d) a light chain CDR1 having the amino acid sequence of SEQ ID NO: 46;
(e) a light chain CDR2 having the amino acid sequence of SEQ ID NO: 47; and
(f) a light chain CDR3 having the amino acid sequence of SEQ ID NO: 48, or
   selected from the group consisting of:
(a) a heavy chain CDR1 having the amino acid sequence of SEQ ID NO: 51;
(b) a heavy chain CDR2 having the amino acid sequence of SEQ ID NO: 52;
(c) a heavy chain CDR3 having the amino acid sequence of SEQ ID NO: 53;
(d) a light chain CDR1 having the amino acid sequence of SEQ ID NO: 54;
(e) a light chain CDR2 having the amino acid sequence of SEQ ID NO: 55; and
(f) a light chain CDR3 having the amino acid sequence of SEQ ID NO: 56, or
   selected from the group consisting of:
(a) a heavy chain CDR1 having the amino acid sequence of SEQ ID NO: 59;
(b) a heavy chain CDR2 having the amino acid sequence of SEQ ID NO: 60;
(c) a heavy chain CDR3 having the amino acid sequence of SEQ ID NO: 61;
(d) a light chain CDR1 having the amino acid sequence of SEQ ID NO: 62;
(e) a light chain CDR2 having the amino acid sequence of SEQ ID NO: 63; and
(f) a light chain CDR3 having the amino acid sequence of SEQ ID NO: 64, or
   selected from the group consisting of:
(a) a heavy chain CDR1 having the amino acid sequence of SEQ ID NO: 67;
(b) a heavy chain CDR2 having the amino acid sequence of SEQ ID NO: 68;
(c) a heavy chain CDR3 having the amino acid sequence of SEQ ID NO: 69;
(d) a light chain CDR1 having the amino acid sequence of SEQ ID NO: 70;
(e) a light chain CDR2 having the amino acid sequence of SEQ ID NO: 71; and
(f) a light chain CDR3 having the amino acid sequence of SEQ ID NO: 72, or
   selected from the group consisting of:
(a) a heavy chain CDR1 having the amino acid sequence of SEQ ID NO: 75;
(b) a heavy chain CDR2 having the amino acid sequence of SEQ ID NO: 76;
(c) a heavy chain CDR3 having the amino acid sequence of SEQ ID NO: 77;
(d) a light chain CDR1 having the amino acid sequence of SEQ ID NO: 78;
(e) a light chain CDR2 having the amino acid sequence of SEQ ID NO: 79; and
(f) a light chain CDR3 having the amino acid sequence of SEQ ID NO: 80, or
a conservative substitution variant of one or more of (a)-(f), or
an antibody, antibody reagent, or antigen-binding portion thereof comprising the heavy chain variable region sequence of SEQ ID NO: 9, or
an antibody, antibody reagent, or antigen-binding portion thereof comprising the light chain variable region sequence of SEQ ID NO: 10, or
an antibody, antibody reagent, or antigen-binding portion thereof comprising the heavy chain variable region sequence of SEQ ID NO: 25, or
   an antibody, antibody reagent, or antigen-binding portion thereof comprising the light chain variable region sequence of SEQ ID NO: 26, or
an antibody, antibody reagent, or antigen-binding portion thereof comprising the heavy chain variable region sequence of SEQ ID NO: 33, or
an antibody, antibody reagent, or antigen-binding portion thereof comprising the light chain variable region sequence of SEQ ID NO: 34, or
an antibody, antibody reagent, or antigen-binding portion thereof comprising the heavy chain variable region sequence of SEQ ID NO: 41, or
an antibody, antibody reagent, or antigen-binding portion thereof comprising the light chain variable region sequence of SEQ ID NO: 42, or
an antibody, antibody reagent, or antigen-binding portion thereof comprising the heavy chain variable region sequence of SEQ ID NO: 49, or
an antibody, antibody reagent, or antigen-binding portion thereof comprising the light chain variable region sequence of SEQ ID NO: 50, or
an antibody, antibody reagent, or antigen-binding portion thereof comprising the heavy chain variable region sequence of SEQ ID NO: 57, or
an antibody, antibody reagent, or antigen-binding portion thereof comprising the light chain variable region sequence of SEQ ID NO: 58, or
an antibody, antibody reagent, or antigen-binding portion thereof comprising the heavy chain variable region sequence of SEQ ID NO: 65, or
an antibody, antibody reagent, or antigen-binding portion thereof comprising the light chain variable region sequence of SEQ ID NO: 66, or
an antibody, antibody reagent, or antigen-binding portion thereof comprising the heavy chain variable region sequence of SEQ ID NO: 73, or
an antibody, antibody reagent, or antigen-binding portion thereof comprising the light chain variable region sequence of SEQ ID NO: 74; or
(ii) the composition kit, or combination as described herein; or
(iii) the cell as described herein, to the subject.

In some embodiments of any of the aspects, the autoimmune disorder or the autoinflammatory disorder is a T-cell mediated disorder. In some embodiments of any of the aspects, the autoimmune disorder or the autoinflammatory disorder is selected from the group consisting of: type-1 diabetes, rheumatoid arthritis, multiple sclerosis, celiac disease, Rasmussen's encephalitis, hypothyroidism, Addison's disease, and amyotrophic lateral sclerosis (ALS).

In some embodiments of any of the aspects, described herein is the antibody, antibody reagent, antigen-binding fragment thereof, or CAR as described herein, or an antibody, antibody reagent, antigen-binding portion thereof, or CAR comprising at least one heavy or light chain complementarity determining region (CDR) selected from the group consisting of:
(a) a heavy chain CDR1 having the amino acid sequence of SEQ ID NO: 11;
(b) a heavy chain CDR2 having the amino acid sequence of SEQ ID NO: 12;
(c) a heavy chain CDR3 having the amino acid sequence of SEQ ID NO: 13;
(d) a light chain CDR1 having the amino acid sequence of SEQ ID NO: 14;
(e) a light chain CDR2 having the amino acid sequence of SEQ ID NO: 15; and
(f) a light chain CDR3 having the amino acid sequence of SEQ ID NO: 16, or
   selected from the group consisting of:
(a) a heavy chain CDR1 having the amino acid sequence of SEQ ID NO: 27;
(b) a heavy chain CDR2 having the amino acid sequence of SEQ ID NO: 28;
(c) a heavy chain CDR3 having the amino acid sequence of SEQ ID NO: 29;
(d) a light chain CDR1 having the amino acid sequence of SEQ ID NO: 30;
(e) a light chain CDR2 having the amino acid sequence of SEQ ID NO: 31; and
(f) a light chain CDR3 having the amino acid sequence of SEQ ID NO: 32, or
   selected from the group consisting of:
(a) a heavy chain CDR1 having the amino acid sequence of SEQ ID NO: 35;
(b) a heavy chain CDR2 having the amino acid sequence of SEQ ID NO: 36;
(c) a heavy chain CDR3 having the amino acid sequence of SEQ ID NO: 37;
(d) a light chain CDR1 having the amino acid sequence of SEQ ID NO: 38;
(e) a light chain CDR2 having the amino acid sequence of SEQ ID NO: 39; and
(f) a light chain CDR3 having the amino acid sequence of SEQ ID NO: 40, or
   selected from the group consisting of:
(a) a heavy chain CDR1 having the amino acid sequence of SEQ ID NO: 43;
(b) a heavy chain CDR2 having the amino acid sequence of SEQ ID NO: 44;
(c) a heavy chain CDR3 having the amino acid sequence of SEQ ID NO: 45;
(d) a light chain CDR1 having the amino acid sequence of SEQ ID NO: 46;
(e) a light chain CDR2 having the amino acid sequence of SEQ ID NO: 47; and
(f) a light chain CDR3 having the amino acid sequence of SEQ ID NO: 48, or
   selected from the group consisting of:
(a) a heavy chain CDR1 having the amino acid sequence of SEQ ID NO: 51;
(b) a heavy chain CDR2 having the amino acid sequence of SEQ ID NO: 52;
(c) a heavy chain CDR3 having the amino acid sequence of SEQ ID NO: 53;
(d) a light chain CDR1 having the amino acid sequence of SEQ ID NO: 54;
(e) a light chain CDR2 having the amino acid sequence of SEQ ID NO: 55; and
(f) a light chain CDR3 having the amino acid sequence of SEQ ID NO: 56, or
   selected from the group consisting of:
(a) a heavy chain CDR1 having the amino acid sequence of SEQ ID NO: 59;
(b) a heavy chain CDR2 having the amino acid sequence of SEQ ID NO: 60;
(c) a heavy chain CDR3 having the amino acid sequence of SEQ ID NO: 61;
(d) a light chain CDR1 having the amino acid sequence of SEQ ID NO: 62;
(e) a light chain CDR2 having the amino acid sequence of SEQ ID NO: 63; and
(f) a light chain CDR3 having the amino acid sequence of SEQ ID NO: 64, or
   selected from the group consisting of:
(a) a heavy chain CDR1 having the amino acid sequence of SEQ ID NO: 67;
(b) a heavy chain CDR2 having the amino acid sequence of SEQ ID NO: 68;
(c) a heavy chain CDR3 having the amino acid sequence of SEQ ID NO: 69;
(d) a light chain CDR1 having the amino acid sequence of SEQ ID NO: 70;
(e) a light chain CDR2 having the amino acid sequence of SEQ ID NO: 71; and
(f) a light chain CDR3 having the amino acid sequence of SEQ ID NO: 72, or
   selected from the group consisting of:
(a) a heavy chain CDR1 having the amino acid sequence of SEQ ID NO: 75;
(b) a heavy chain CDR2 having the amino acid sequence of SEQ ID NO: 76;
(c) a heavy chain CDR3 having the amino acid sequence of SEQ ID NO: 77;
(d) a light chain CDR1 having the amino acid sequence of SEQ ID NO: 78;
(e) a light chain CDR2 having the amino acid sequence of SEQ ID NO: 79; and
(f) a light chain CDR3 having the amino acid sequence of SEQ ID NO: 80, or
a conservative substitution variant of one or more of (a)-(f); or
an antibody, antibody reagent, or antigen-binding portion thereof comprising the heavy chain variable region sequence of SEQ ID NO: 9, or
an antibody, antibody reagent, or antigen-binding portion thereof comprising the light chain variable region sequence of SEQ ID NO: 10, or
an antibody, antibody reagent, or antigen-binding portion thereof comprising the heavy chain variable region sequence of SEQ ID NO: 25, or
an antibody, antibody reagent, or antigen-binding portion thereof comprising the light chain variable region sequence of SEQ ID NO: 26, or
an antibody, antibody reagent, or antigen-binding portion thereof comprising the heavy chain variable region sequence of SEQ ID NO: 33, or
an antibody, antibody reagent, or antigen-binding portion thereof comprising the light chain variable region sequence of SEQ ID NO: 34, or
an antibody, antibody reagent, or antigen-binding portion thereof comprising the heavy chain variable region sequence of SEQ ID NO: 41, or
an antibody, antibody reagent, or antigen-binding portion thereof comprising the light chain variable region sequence of SEQ ID NO: 42, or
an antibody, antibody reagent, or antigen-binding portion thereof comprising the heavy chain variable region sequence of SEQ ID NO: 49, or
an antibody, antibody reagent, or antigen-binding portion thereof comprising the light chain variable region sequence of SEQ ID NO: 50, or
an antibody, antibody reagent, or antigen-binding portion thereof comprising the heavy chain variable region sequence of SEQ ID NO: 57, or
an antibody, antibody reagent, or antigen-binding portion thereof comprising the light chain variable region sequence of SEQ ID NO: 58, or
an antibody, antibody reagent, or antigen-binding portion thereof comprising the heavy chain variable region sequence of SEQ ID NO: 65, or
an antibody, antibody reagent, or antigen-binding portion thereof comprising the light chain variable region sequence of SEQ ID NO: 66, or
an antibody, antibody reagent, or antigen-binding portion thereof comprising the heavy chain variable region sequence of SEQ ID NO: 73, or
an antibody, antibody reagent, or antigen-binding portion thereof comprising the light chain variable region sequence of SEQ ID NO: 74; or
the composition, kit, or combination as described herein; or the cell as described herein, for use in a method of treating an autoimmune disorder or an auto-inflammatory disorder in a subject in need thereof.

In some embodiments of any of the aspects, described herein is a method of suppressing an immune response or an inflammatory response in a subject in need thereof, the method comprising administering (i) the antibody, antibody reagent, antigen-binding portion thereof, or CAR as disclosed herein, or an antibody, antibody reagent, antigen-binding portion thereof, or CAR comprising at least one heavy or light chain complementarity determining region (CDR) selected from the group consisting of:
(a) a heavy chain CDR1 having the amino acid sequence of SEQ ID NO: 11;
(b) a heavy chain CDR2 having the amino acid sequence of SEQ ID NO: 12;
(c) a heavy chain CDR3 having the amino acid sequence of SEQ ID NO: 13;
(d) a light chain CDR1 having the amino acid sequence of SEQ ID NO: 14;
(e) a light chain CDR2 having the amino acid sequence of SEQ ID NO: 15; and
(f) a light chain CDR3 having the amino acid sequence of SEQ ID NO: 16, or
selected from the group consisting of:
(a) a heavy chain CDR1 having the amino acid sequence of SEQ ID NO: 27;
(b) a heavy chain CDR2 having the amino acid sequence of SEQ ID NO: 28;
(c) a heavy chain CDR3 having the amino acid sequence of SEQ ID NO: 29;
(d) a light chain CDR1 having the amino acid sequence of SEQ ID NO: 30;
(e) a light chain CDR2 having the amino acid sequence of SEQ ID NO: 31; and
(f) a light chain CDR3 having the amino acid sequence of SEQ ID NO: 32, or
   selected from the group consisting of:
(a) a heavy chain CDR1 having the amino acid sequence of SEQ ID NO: 35;
(b) a heavy chain CDR2 having the amino acid sequence of SEQ ID NO: 36;
(c) a heavy chain CDR3 having the amino acid sequence of SEQ ID NO: 37;
(d) a light chain CDR1 having the amino acid sequence of SEQ ID NO: 38;
(e) a light chain CDR2 having the amino acid sequence of SEQ ID NO: 39; and
(f) a light chain CDR3 having the amino acid sequence of SEQ ID NO: 40, or
   selected from the group consisting of:
(a) a heavy chain CDR1 having the amino acid sequence of SEQ ID NO: 43;
(b) a heavy chain CDR2 having the amino acid sequence of SEQ ID NO: 44;
(c) a heavy chain CDR3 having the amino acid sequence of SEQ ID NO: 45;
(d) a light chain CDR1 having the amino acid sequence of SEQ ID NO: 46;
(e) a light chain CDR2 having the amino acid sequence of SEQ ID NO: 47; and
(f) a light chain CDR3 having the amino acid sequence of SEQ ID NO: 48, or
   selected from the group consisting of:
(a) a heavy chain CDR1 having the amino acid sequence of SEQ ID NO: 51;
(b) a heavy chain CDR2 having the amino acid sequence of SEQ ID NO: 52;
(c) a heavy chain CDR3 having the amino acid sequence of SEQ ID NO: 53;
(d) a light chain CDR1 having the amino acid sequence of SEQ ID NO: 54;
(e) a light chain CDR2 having the amino acid sequence of SEQ ID NO: 55; and
(f) a light chain CDR3 having the amino acid sequence of SEQ ID NO: 56, or
   selected from the group consisting of:
(a) a heavy chain CDR1 having the amino acid sequence of SEQ ID NO: 59;
(b) a heavy chain CDR2 having the amino acid sequence of SEQ ID NO: 60;
(c) a heavy chain CDR3 having the amino acid sequence of SEQ ID NO: 61;
(d) a light chain CDR1 having the amino acid sequence of SEQ ID NO: 62;
(e) a light chain CDR2 having the amino acid sequence of SEQ ID NO: 63; and
(f) a light chain CDR3 having the amino acid sequence of SEQ ID NO: 64, or
   selected from the group consisting of:
(a) a heavy chain CDR1 having the amino acid sequence of SEQ ID NO: 67;
(b) a heavy chain CDR2 having the amino acid sequence of SEQ ID NO: 68;
(c) a heavy chain CDR3 having the amino acid sequence of SEQ ID NO: 69;
(d) a light chain CDR1 having the amino acid sequence of SEQ ID NO: 70;
(e) a light chain CDR2 having the amino acid sequence of SEQ ID NO: 71; and
(f) a light chain CDR3 having the amino acid sequence of SEQ ID NO: 72, or
   selected from the group consisting of:
(a) a heavy chain CDR1 having the amino acid sequence of SEQ ID NO: 75;
(b) a heavy chain CDR2 having the amino acid sequence of SEQ ID NO: 76;
(c) a heavy chain CDR3 having the amino acid sequence of SEQ ID NO: 77;
(d) a light chain CDR1 having the amino acid sequence of SEQ ID NO: 78;
(e) a light chain CDR2 having the amino acid sequence of SEQ ID NO: 79; and
(f) a light chain CDR3 having the amino acid sequence of SEQ ID NO: 80, or
a conservative substitution variant of one or more of (a)-(f), or
an antibody, antibody reagent, or antigen-binding portion thereof comprising the heavy chain variable region sequence of SEQ ID NO: 9, or
an antibody, antibody reagent, or antigen-binding portion thereof comprising the light chain variable region sequence of SEQ ID NO: 10, or
an antibody, antibody reagent, or antigen-binding portion thereof comprising the heavy chain variable region sequence of SEQ ID NO: 25, or
an antibody, antibody reagent, or antigen-binding portion thereof comprising the light chain variable region sequence of SEQ ID NO: 26, or
an antibody, antibody reagent, or antigen-binding portion thereof comprising the heavy chain variable region sequence of SEQ ID NO: 33, or
an antibody, antibody reagent, or antigen-binding portion thereof comprising the light chain variable region sequence of SEQ ID NO: 34, or
an antibody, antibody reagent, or antigen-binding portion thereof comprising the heavy chain variable region sequence of SEQ ID NO: 41, or
an antibody, antibody reagent, or antigen-binding portion thereof comprising the light chain variable region sequence of SEQ ID NO: 42, or
an antibody, antibody reagent, or antigen-binding portion thereof comprising the heavy chain variable region sequence of SEQ ID NO: 49, or
an antibody, antibody reagent, or antigen-binding portion thereof comprising the light chain variable region sequence of SEQ ID NO: 50, or
an antibody, antibody reagent, or antigen-binding portion thereof comprising the heavy chain variable region sequence of SEQ ID NO: 57, or
an antibody, antibody reagent, or antigen-binding portion thereof comprising the light chain variable region sequence of SEQ ID NO: 58, or
an antibody, antibody reagent, or antigen-binding portion thereof comprising the heavy chain variable region sequence of SEQ ID NO: 65, or
an antibody, antibody reagent, or antigen-binding portion thereof comprising the light chain variable region sequence of SEQ ID NO: 66, or
an antibody, antibody reagent, or antigen-binding portion thereof comprising the heavy chain variable region sequence of SEQ ID NO: 73, or
an antibody, antibody reagent, or antigen-binding portion thereof comprising the light chain variable region sequence of SEQ ID NO: 74; or
(ii) the composition kit, or combination as disclosed herein; or
(iii) the cell as disclosed herein, to the subject.

In some embodiments of any of the aspects, the immune response or the inflammatory response is a T-cell mediated response. In some embodiments of any of the aspects, the immune response or the inflammatory response is associated with the group consisting of: type-1 diabetes, rheumatoid arthritis, multiple sclerosis, celiac disease, Rasmussen's encephalitis, hypothyroidism, Addison's disease, and amyotrophic lateral sclerosis (ALS).

In some embodiments of any of the aspects, described herein is the antibody, antibody reagent, antigen-binding fragment thereof, or CAR as disclosed herein, or an antibody, antibody reagent, antigen-binding portion thereof, or CAR comprising at least one heavy or light chain complementarity determining region (CDR) selected from the group consisting of:
(a) a heavy chain CDR1 having the amino acid sequence of SEQ ID NO: 11;
(b) a heavy chain CDR2 having the amino acid sequence of SEQ ID NO: 12;
(c) a heavy chain CDR3 having the amino acid sequence of SEQ ID NO: 13;
(d) a light chain CDR1 having the amino acid sequence of SEQ ID NO: 14;
(e) a light chain CDR2 having the amino acid sequence of SEQ ID NO: 15; and
(f) a light chain CDR3 having the amino acid sequence of SEQ ID NO: 16, or
   selected from the group consisting of:
(a) a heavy chain CDR1 having the amino acid sequence of SEQ ID NO: 27;
(b) a heavy chain CDR2 having the amino acid sequence of SEQ ID NO: 28;
(c) a heavy chain CDR3 having the amino acid sequence of SEQ ID NO: 29;
(d) a light chain CDR1 having the amino acid sequence of SEQ ID NO: 30;
(e) a light chain CDR2 having the amino acid sequence of SEQ ID NO: 31; and
(f) a light chain CDR3 having the amino acid sequence of SEQ ID NO: 32, or
   selected from the group consisting of:
(a) a heavy chain CDR1 having the amino acid sequence of SEQ ID NO: 35;
(b) a heavy chain CDR2 having the amino acid sequence of SEQ ID NO: 36;
(c) a heavy chain CDR3 having the amino acid sequence of SEQ ID NO: 37;
(d) a light chain CDR1 having the amino acid sequence of SEQ ID NO: 38;
(e) a light chain CDR2 having the amino acid sequence of SEQ ID NO: 39; and
(f) a light chain CDR3 having the amino acid sequence of SEQ ID NO: 40, or
   selected from the group consisting of:
(a) a heavy chain CDR1 having the amino acid sequence of SEQ ID NO: 43;
(b) a heavy chain CDR2 having the amino acid sequence of SEQ ID NO: 44;
(c) a heavy chain CDR3 having the amino acid sequence of SEQ ID NO: 45;
(d) a light chain CDR1 having the amino acid sequence of SEQ ID NO: 46;
(e) a light chain CDR2 having the amino acid sequence of SEQ ID NO: 47; and
(f) a light chain CDR3 having the amino acid sequence of SEQ ID NO: 48, or
   selected from the group consisting of:
(a) a heavy chain CDR1 having the amino acid sequence of SEQ ID NO: 51;
(b) a heavy chain CDR2 having the amino acid sequence of SEQ ID NO: 52;
(c) a heavy chain CDR3 having the amino acid sequence of SEQ ID NO: 53;
(d) a light chain CDR1 having the amino acid sequence of SEQ ID NO: 54;
(e) a light chain CDR2 having the amino acid sequence of SEQ ID NO: 55; and
(f) a light chain CDR3 having the amino acid sequence of SEQ ID NO: 56, or
   selected from the group consisting of:
(a) a heavy chain CDR1 having the amino acid sequence of SEQ ID NO: 59;
(b) a heavy chain CDR2 having the amino acid sequence of SEQ ID NO: 60;
(c) a heavy chain CDR3 having the amino acid sequence of SEQ ID NO: 61;
(d) a light chain CDR1 having the amino acid sequence of SEQ ID NO: 62;
(e) a light chain CDR2 having the amino acid sequence of SEQ ID NO: 63; and
(f) a light chain CDR3 having the amino acid sequence of SEQ ID NO: 64, or
   selected from the group consisting of:
(a) a heavy chain CDR1 having the amino acid sequence of SEQ ID NO: 67;
(b) a heavy chain CDR2 having the amino acid sequence of SEQ ID NO: 68;
(c) a heavy chain CDR3 having the amino acid sequence of SEQ ID NO: 69;
(d) a light chain CDR1 having the amino acid sequence of SEQ ID NO: 70;
(e) a light chain CDR2 having the amino acid sequence of SEQ ID NO: 71; and
(f) a light chain CDR3 having the amino acid sequence of SEQ ID NO: 72, or
   selected from the group consisting of:
(a) a heavy chain CDR1 having the amino acid sequence of SEQ ID NO: 75;
(b) a heavy chain CDR2 having the amino acid sequence of SEQ ID NO: 76;
(c) a heavy chain CDR3 having the amino acid sequence of SEQ ID NO: 77;
(d) a light chain CDR1 having the amino acid sequence of SEQ ID NO: 78;
(e) a light chain CDR2 having the amino acid sequence of SEQ ID NO: 79; and
(f) a light chain CDR3 having the amino acid sequence of SEQ ID NO: 80, or
a conservative substitution variant of one or more of (a)-(f); or
an antibody, antibody reagent, or antigen-binding portion thereof comprising the heavy chain variable region sequence of SEQ ID NO: 9, or
an antibody, antibody reagent, or antigen-binding portion thereof comprising the light chain variable region sequence of SEQ ID NO: 10, or
an antibody, antibody reagent, or antigen-binding portion thereof comprising the heavy chain variable region sequence of SEQ ID NO: 25, or
an antibody, antibody reagent, or antigen-binding portion thereof comprising the light chain variable region sequence of SEQ ID NO: 26, or
an antibody, antibody reagent, or antigen-binding portion thereof comprising the heavy chain variable region sequence of SEQ ID NO: 33, or
an antibody, antibody reagent, or antigen-binding portion thereof comprising the light chain variable region sequence of SEQ ID NO: 34, or
an antibody, antibody reagent, or antigen-binding portion thereof comprising the heavy chain variable region sequence of SEQ ID NO: 41, or
an antibody, antibody reagent, or antigen-binding portion thereof comprising the light chain variable region sequence of SEQ ID NO: 42, or
an antibody, antibody reagent, or antigen-binding portion thereof comprising the heavy chain variable region sequence of SEQ ID NO: 49, or
an antibody, antibody reagent, or antigen-binding portion thereof comprising the light chain variable region sequence of SEQ ID NO: 50, or
an antibody, antibody reagent, or antigen-binding portion thereof comprising the heavy chain variable region sequence of SEQ ID NO: 57, or
an antibody, antibody reagent, or antigen-binding portion thereof comprising the light chain variable region sequence of SEQ ID NO: 58, or
an antibody, antibody reagent, or antigen-binding portion thereof comprising the heavy chain variable region sequence of SEQ ID NO: 65, or
an antibody, antibody reagent, or antigen-binding portion thereof comprising the light chain variable region sequence of SEQ ID NO: 66, or
an antibody, antibody reagent, or antigen-binding portion thereof comprising the heavy chain variable region sequence of SEQ ID NO: 73, or
an antibody, antibody reagent, or antigen-binding portion thereof comprising the light chain variable region sequence of SEQ ID NO: 74; or
the composition, kit, or combination as disclosed herein; or the cell as disclosed herein, for use in a method suppressing an immune response or an inflammatory response in a subject in need thereof.

Additionally described herein are the development and characterization of anti-PD1 antibodies demonstrated to have high specificity and binding affinity. These antibodies are developed using a system which harnesses the natural affinity maturation processes in order to provide new therapeutic antibodies with significantly improved affinity and specificity.

In one aspect of any of the embodiments, described herein is an antibody, antibody reagent, antigen-binding fragment thereof, or chimeric antigen receptor (CAR), that specifically binds an PD1 polypeptide, said antibody reagent, antigen-binding portion thereof, or CAR comprising at least one heavy or light chain complementarity determining region (CDR) selected from the group consisting of:
(a) a heavy chain CDR1 having the amino acid sequence of SEQ ID NO: 83;
(b) a heavy chain CDR2 having the amino acid sequence of SEQ ID NO: 84;
(c) a heavy chain CDR3 having the amino acid sequence of SEQ ID NO: 85;
(d) a light chain CDR1 having the amino acid sequence of SEQ ID NO: 86;
(e) a light chain CDR2 having the amino acid sequence of SEQ ID NO: 87; and
(f) a light chain CDR3 having the amino acid sequence of SEQ ID NO: 88, or
a conservative substitution variant of one or more of (a)-(f).

In some embodiments of any of the aspects, the antibody, antibody reagent, antigen-binding portion thereof, or CAR includes heavy chain CDRs having the amino acid sequences of SEQ ID NOs: 83-85, or a conservative substitution variant of such amino acid sequence. In some embodiments of any of the aspects, the antibody, antibody reagent, antigen-binding portion thereof, or CAR includes light chain CDRs having the amino acid sequences of SEQ ID NOs: 86-88, or a conservative substitution variant of such amino acid sequence. In some embodiments of any of the aspects, the antibody, antibody reagent, antigen-binding portion thereof, or CAR includes: heavy chain CDRs having the amino acid sequences of SEQ ID NOs: 83-85 and light chain CDRs having the amino acid sequences of SEQ ID NOs: 86-88 or a conservative substitution variant of such amino acid sequence.

In one aspect of any of the embodiments, described herein is a first antibody, antibody reagent, antigen-binding portion thereof, or CAR that specifically binds an PD1 polypeptide, and can compete for binding of PD1 with a second antibody comprising: heavy chain CDRs having the amino acid sequences of SEQ ID NOs: 83-85 and light chain CDRs having the amino acid sequences of SEQ ID NOs: 86-88 or a conservative substitution variant of such amino acid sequence.

In some embodiments of any of the aspects, the first antibody, antibody reagent, antigen-binding fragment thereof, or chimeric antigen receptor (CAR) includes at least one heavy or light chain complementarity determining region (CDR) selected from the group consisting of:
(a) a heavy chain CDR1 having the amino acid sequence of SEQ ID NO: 83;
(b) a heavy chain CDR2 having the amino acid sequence of SEQ ID NO: 84;
(c) a heavy chain CDR3 having the amino acid sequence of SEQ ID NO: 85;
(d) a light chain CDR1 having the amino acid sequence of SEQ ID NO: 86;
(e) a light chain CDR2 having the amino acid sequence of SEQ ID NO: 87; and
(f) a light chain CDR3 having the amino acid sequence of SEQ ID NO: 88; or
a conservative substitution variant of one or more of (a)-(f).

In some embodiments of any of the aspects, the antibody, antibody reagent, antigen-binding portion thereof, or CAR includes the heavy chain variable region sequence of SEQ ID NO: 81. In some embodiments of any of the aspects, the antibody, antibody reagent, antigen-binding portion thereof, or CAR includes the light chain variable region sequence of SEQ ID NO: 82. In some embodiments of any of the aspects, the antibody, antibody reagent, antigen-binding portion thereof, or CAR includes: the heavy chain variable region sequence of SEQ ID NO: 81 and the light chain variable region sequence of SEQ ID NO: 82.

In one aspect of any of the embodiments, described herein is a composition comprising the antibody, antibody reagent, antigen-binding portion thereof, or CAR as described herein, and a chemotherapeutic agent. In some embodiments of any of the aspects, the antibody, antibody reagent, or antigen-binding portion thereof is conjugated to the chemotherapeutic agent.

In one aspect of any of the embodiments, described herein is method of treating cancer in a subject in need thereof, the method comprising administering the antibody, antibody reagent, antigen-binding fragment thereof, or CAR as described herein; or the composition as described herein; or the cell as described herein, to the subject. In some embodiments of any of the aspects, the cancer is selected from the group consisting of: Non-small cell lung cancer; melanoma; metastatic melanoma; renal cell carcinoma; squamous cell carcinoma of the head and neck; Hodgkin lymphoma; classical Hodgkin lymphoma; and urothelial carcinoma.

### BRIEF DESCRIPTION OF THE DRAWINGS

FIG. 1A is a diagram of the IgH and Igk loci of a first mouse model that was used to diversify anti-PD1 antibody, 17D8, according to some implementations of the present disclosure.

FIG. 1B is a diagram of the IgH and Igk loci of a second mouse model that was used to diversify anti-PD1 antibody, 17D8, according to some implementations of the present disclosure.

FIG. 2 depicts the effect of various antibodies on the interaction between PD1 and PD-L1, according to some implementations of the present disclosure.

FIG. 3A depicts FACS analysis of B cells in blood from wild-type 129/Sv mouse and mouse model 1, according to some implementations of the present disclosure.

FIG. 3B depicts FACS analysis of B cells in blood from wild-type 129/Sv mouse and mouse model 2, according to some implementations of the present disclosure.

FIG. 3C depicts the percentage of IgH rearrangements involving the 17D8 V_{H} and DJ_{H} segments in mouse model 1, according to some implementations of the present disclosure.

FIG. 3D depicts the fraction of Igk transcripts corresponding to 17D8LC in mouse model 1, according to some implementations of the present disclosure.

FIG. 3E depicts the percentage of IgH rearrangements involving the 17D8 V_{H}, mouse D and J_{H} segments in mouse model 2, according to some implementations of the present disclosure.

FIG. 3F depicts the fraction of Igk transcripts corresponding to 17D8LC in mouse model 2, according to some implementations of the present disclosure.

FIG. 4A depicts ELISA detection of anti-PD1 IgG in plasma after immunization of mouse model 1, according to some implementations of the present disclosure.

FIG. 4B depicts ELISA detection of anti-PD1 IgG in plasma after immunization of mouse model 2, according to some implementations of the present disclosure.

FIG. 4C depicts sorting of PD1-binding B cells from immunized mouse model 1 and cloning of 17D8 antibody or its variants, according to some implementations of the present disclosure.

FIG. 4D depicts profiles of new CDR H3s in 17D8 antibody variants from mouse model 1, according to some implementations of the present disclosure.

FIG. 4E depicts sorting of PD1-binding B cells from immunized mouse model 2 and cloning of 17D8 antibody variants, according to some implementations of the present disclosure.

FIG. 4F depicts profiles of new CDR H3s in 17D8 antibody variants from mouse model 2, according to some implementations of the present disclosure. FIG. 4G depicts frequencies of amino acid changes in the 17D8 antibody heavy chains with the original CDR H3 and new CDR H3s from mouse model 1, according to some implementations of the present disclosure.

FIG. 4H depicts frequencies of amino acid changes in the heavy chains of 17D8 antibodies with new CDR H3s in mouse model 2, according to some implementations of the present disclosure.

FIG. 5A depicts sequence comparisons of three new anti-PD1 antibodies from Mouse Model 1 with the 17D8 antibody and Nivolumab, according to some implementations of the present disclosure. FIG. 5A discloses SEQ ID NOS 125-126, 81, 41, 9, 127-128, 82, 42, and 10, respectively, in order of appearance.

FIG. 5B depicts sequence comparisons of two new anti-PD1 antibodies from Mouse Model 2 with the 17D8 antibody and Nivolumab, according to some implementations of the present disclosure. FIG. 5B discloses SEQ ID NOS 125-126, 73, 1, 127-128 74, and 2, respectively, in order of appearance.

FIG. 5C depicts ELISA analysis of PD1-binding activities of 17D8 antibody, Nivolumab, Pembrolizumab and five new anti-PD1 antibodies isolated from Mouse Models 1 and 2, according to some implementations of the present disclosure.

FIG. 5D depicts Biacore analysis of binding kinetics of select anti-PD1 antibodies, according to some implementations of the present disclosure.

FIG. 5E depicts ELISA analysis of the same antibodies in FIG. 5C, but with PD1-Fc fusion as the coating antigen on ELISA plate, according to some implementations of the present disclosure.

FIG. 5F depicts FACS analysis of binding of anti-PD1 antibodies to PD1 expressed on cell surface, according to some implementations of the present disclosure.

FIG. 5G depicts ELISA analysis of the same antibodies in FIG. 5C, but with PD1 N-terminal-GST fusion protein as the coating antigen on ELISA plate, according to some implementations of the present disclosure.

FIG. 6A depicts FACS analysis of the effects of anti-PD1 antibodies on PD1/PD-L1 interaction, according to some implementations of the present disclosure.

FIG. 6B depicts FACS analysis of the effects of anti-PD1 antibodies on PD1/PD-L2 interaction, according to some implementations of the present disclosure.

FIG. 7A depicts alignment of M1-1 to M1-7HCs with the 17D8HC, according to some implementations of the present disclosure. FIG. 7A discloses SEQ ID NOS 129 and 101-107, respectively, in order of appearance.

FIG. 7B depicts alignment of M1-1 to M1-7LCs with the 17D8LC, according to some implementations of the present disclosure. FIG. 7B discloses SEQ ID NOS 130 and 113-119, respectively, in order of appearance.

FIG. 7C depicts alignment of M2-1 to M2-5HCs with the 17D8HC, according to some implementations of the present disclosure. FIG. 7C discloses SEQ ID NOS 129 and 108-112, respectively, in order of appearance.

FIG. 7D depicts alignment of M2-1 to M2-5LCs with the 17D8LC, according to some implementations of the present disclosure. FIG. 7D discloses SEQ ID NOS 130 and 120-124, respectively, in order of appearance.

FIGS. 8A-8F depict analysis of additional antibodies isolated from Mouse Models 1 and supplement FIGS. 5A-5G, according to some implementations of the present disclosure. FIG. 8A discloses SEQ ID NOS 125, 17, 49, 25, 57, 127, 18, 50, 26, and 58, and FIG. 8B discloses SEQ ID NOS 125, 65, 131, 33, 127, 66, 132, and 34, respectively, in order of appearance.

FIG. 9A-9B depict effects of additional antibodies isolated from Mouse Model 1 and 2 and supplement FIGS. 6A-6B, according to some implementations of the present disclosure.

### DETAILED DESCRIPTION

Some aspects of the present disclosure relate to high-affinity humanized anti-PD1 antibodies that inhibit, rather than activate, T-cell responses. For example, in some such embodiments, described herein are antibodies, antibody reagents, antigen-binding fragments thereof, or chimeric antigen receptors (CARs) that increase PD-1/PD-L1 binding in vitro. Such antibodies, antigen binding portions thereof, etc., can permit treatment of autoimmune and/or autoinflammatory disorders. In some embodiments, the technology described herein relates to chimeric antigen receptors (CARs) and CAR-T therapy for autoimmune and/or autoinflammatory disorders. In some embodiments, the technology described herein relates to monoclonal antibody therapy for autoimmune and/or autoinflammatory disorders. In some embodiments, the technology described herein relates to antibody-drug conjugates for the treatment of autoimmune and/or autoinflammatory disorders.

As used herein, “PD1” or “programmed death 1” refers to a cell surface receptor that suppresses T-cell inflammatory responses. PD1 serves as an immune system checkpoint and prevents against development of autoimmune diseases. In some implementations, anti-PD1 therapies having the usual antagonizing effect on the antigen (PD1 in this case) are used in cancer treatments to stimulate the immune system. In some other implementations, such as what are disclosed herein, anti-PD1 therapies having the opposite agonizing effect can be used in autoimmune or autoinflammatory treatments to suppress the immune system. The sequences of PD1 expression products are known for a number of species, e.g., human PD1 (NCBI Gene ID No: 5133) mRNA (NCBI Ref Seq: NM_005018.2) and polypeptide (NCBI Ref Seq: NP_005009.2).

As used herein, the term “antibody” refers to immunoglobulin molecules and immunologically active portions of immunoglobulin molecules, i.e., molecules that contain an antigen binding site that immunospecifically binds an antigen. The term also refers to antibodies comprised of two immunoglobulin heavy chains and two immunoglobulin light chains as well as a variety of forms including full length antibodies and antigen-binding portions thereof; including, for example, an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding portion thereof, and/or bifunctional hybrid antibodies.

Each heavy chain is composed of a variable region of said heavy chain (abbreviated here as HCVR or VH) and a constant region of said heavy chain. The heavy chain constant region consists of three domains CH1, CH2 and CH3. Each light chain is composed of a variable region of said light chain (abbreviated here as LCVR or VL) and a constant region of said light chain. The light chain constant region consists of a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs which are arranged from the N terminus to the C terminus in the following order: FR1, CDR1, FR2, CDR2, FR3, CDR3, FR4. This structure is well known to those skilled in the art.

As used herein, the term “CDR” refers to the complementarity determining regions within antibody variable sequences. The exact boundaries of these CDRs have been defined differently according to different systems. CDRs may be defined according to the Kabat system (see Kabat, E. A. et al., 1991, “Sequences of Proteins of Immunological Interest”, 5th edit., NIH Publication no. 91-3242, U.S. Department of Health and Human Services). Other systems may be used to define CDRs, which as the system devised by Chothia et al (see Chothia, C. & Lesk, A. M., 1987, “Canonical structures for the hypervariable regions of immunoglobulins”, J. Mol. Biol., 196, 901-917) and the IMGT system (see Lefranc, M. P., 1997, “Unique database numbering system for immunogenetic analysis”, Immunol. Today, 18, 50). An antibody typically contains 3 heavy chain CDRs and 3 light chain CDRs. The term CDR or CDRs is used here to indicate one or several of these regions. A person skilled in the art is able to readily compare the different systems of nomenclature and determine whether a particular sequence may be defined as a CDR. The methods and compositions used herein may utilize CDRs defined according to any of these systems. The CDRs disclosed herein were identified via the IMGT system which stands for ImMunoGeneTics, which is an international information system for immunoglobulins and T cell receptors.

The term “antigen-binding portion” of an antibody refers to one or more portions of an antibody as described herein, said one or more portions still having the binding affinities as defined above herein. Portions of a complete antibody have been shown to be able to carry out the antigen-binding function of an antibody. In accordance with the term “antigen-binding portion” of an antibody, examples of binding portions include (i) an Fab portion, i.e., a monovalent portion composed of the VL, VH, CL and CH1 domains; (ii) an F(ab′)2 portion, i.e., a bivalent portion comprising two Fab portions linked to one another in the hinge region via a disulfide bridge; (iii) an Fd portion composed of the VH and CH1 domains; (iv) an Fv portion composed of the FL and VH domains of a single arm of an antibody; and (v) a dAb portion consisting of a VH domain or of VH, CH1, CH2, DH3, or VH, CH2, CH3 (dAbs, or single domain antibodies, comprising only V_{L} domains have also been shown to specifically bind to target epitopes).

Although the two domains of the Fv portion, namely VL and VH, are encoded by separate genes, they may further be linked to one another using a synthetic linker, e.g., a poly-G4S amino acid sequence (‘G4S’) (SEQ ID NO: 98), and recombinant methods, making it possible to prepare them as a single protein chain in which the VL and VH regions combine in order to form monovalent molecules (known as single chain Fv (ScFv)).

The term “antigen-binding portion” of an antibody is also intended to comprise such single chain antibodies. Other forms of single chain antibodies such as “diabodies” can also be included. Diabodies are bivalent, bispecific antibodies in which VH and VL domains are expressed on a single polypeptide chain, but using a linker which is too short for the two domains being able to combine on the same chain, thereby forcing said domains to pair with complementary domains of a different chain and to form two antigen-binding sites. An immunoglobulin constant domain refers to a heavy or light chain constant domain. Human IgG heavy chain and light chain constant domain amino acid sequences are known in the art.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments as well as complete antibodies.

An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

Furthermore, an antibody, antigen-binding portion thereof, or CAR as described herein may be part of a larger immunoadhesion molecule formed by covalent or noncovalent association of said antibody or antibody portion with one or more further proteins or peptides. Relevant to such immunoadhesion molecules are the use of the streptavidin core region in order to prepare a tetrameric scFv molecule and the use of a cystein residue, a marker peptide and a C-terminal polyhistidinyl, e.g., hexahistidinyl tag (SEQ ID NO: 99) (‘hexahistidinyl tag’) in order to produce bivalent and biotinylated scFv molecules.

In some embodiments, the antibody, antibody reagent, antigen-binding portion thereof, or CAR described herein can be an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, a bispecific antibody, and a functionally active epitope-binding portion thereof.

In some embodiments, the antibody or antigen-binding portion thereof is a fully human antibody. In some embodiments, the antibody, antigen-binding portion thereof, is a humanized antibody or antibody reagent. In some embodiments, the antibody, antigen-binding portion thereof, is a fully humanized antibody or antibody reagent. In some embodiments, the antibody or antigen-binding portion thereof, is a chimeric antibody or antibody reagent. In some embodiments, the antibody, antigen-binding portion thereof, is a recombinant polypeptide. In some embodiments, the CAR comprises an extracellular domain that binds PD1, wherein the extracellular domain comprises a humanized or chimeric antibody or antigen-binding portion thereof.

The term “human antibody” refers to antibodies whose variable and constant regions correspond to or are derived from immunoglobulin sequences of the human germ line, as described, for example, by Kabat et al. (see Kabat, et al. (1991) Sequences of Proteins of Immunological Interest, Fifth Edition, U.S. Department of Health and Human Services, NIH Publication No. 91-3242). However, the human antibodies can contain amino acid residues not encoded by human germ line immunoglobulin sequences (for example mutations which have been introduced by random or site-specific mutagenesis in vitro or by somatic mutation in vivo), for example in the CDRs, and in particular in CDR3. Recombinant human antibodies as described herein have variable regions and may also contain constant regions derived from immunoglobulin sequences of the human germ line (see Kabat, E. A., et al. (1991) Sequences of Proteins of Immunological Interest, Fifth Edition, U.S. Department of Health and Human Services, NIH Publication No. 91-3242).

According to particular embodiments, however, such recombinant human antibodies are subjected to in-vitro mutagenesis (or to a somatic in-vivo mutagenesis, if an animal is used which is transgenic due to human Ig sequences) so that the amino acid sequences of the VH and VL regions of the recombinant antibodies are sequences which although related to or derived from VH and VL sequences of the human germ line, do not naturally exist in vivo within the human antibody germ line repertoire. According to particular embodiments, recombinant antibodies of this kind are the result of selective mutagenesis or back mutation or of both. Preferably, mutagenesis leads to an affinity to the target which is greater, and/or an affinity to non-target structures which is smaller than that of the parent antibody.

Generating a humanized antibody from the sequences and information provided herein can be practiced by those of ordinary skill in the art without undue experimentation. In one approach, there are four general steps employed to humanize a monoclonal antibody, see, e.g., U.S. Pat. Nos. 5,585,089; 6,835,823; 6,824,989. These are: (1) determining the nucleotide and predicted amino acid sequence of the starting antibody light and heavy variable domains; (2) designing the humanized antibody, i.e., deciding which antibody framework region to use during the humanizing process; (3) the actual humanizing methodologies/techniques; and (4) the transfection and expression of the humanized antibody.

Usually the CDR regions in humanized antibodies and human antibody variants are substantially identical, and more usually, identical to the corresponding CDR regions in the mouse or human antibody from which they were derived. In some embodiments, it is possible to make one or more conservative amino acid substitutions of CDR residues without appreciably affecting the binding affinity of the resulting humanized immunoglobulin or human antibody variant. In some embodiments, substitutions of CDR regions can enhance binding affinity.

The term “chimeric antibody” refers to antibodies which contain sequences for the variable region of the heavy and light chains from one species and constant region sequences from another species, such as antibodies having murine heavy and light chain variable regions linked to human constant regions. Humanized antibodies have variable region framework residues substantially from a human antibody (termed an acceptor antibody) and complementarity determining regions substantially from a non-human antibody, e.g., a mouse-antibody, (referred to as the donor immunoglobulin).

The constant region(s), if present, are also substantially or entirely from a human immunoglobulin. The human variable domains are usually chosen from human antibodies whose framework sequences exhibit a high degree of sequence identity with the (murine) variable region domains from which the CDRs were derived. The heavy and light chain variable region framework residues can be substantially similar to a region of the same or different human antibody sequences. The human antibody sequences can be the sequences of naturally occurring human antibodies or can be consensus sequences of several human antibodies.

In addition, techniques developed for the production of “chimeric antibodies” by splicing genes from a mouse, or other species, antibody molecule of appropriate antigen specificity together with genes from a human antibody molecule of appropriate biological activity can be used. The variable segments of chimeric antibodies are typically linked to at least a portion of an immunoglobulin constant region (Fc), typically that of a human immunoglobulin. Human constant region DNA sequences can be isolated in accordance with well-known procedures from a variety of human cells, such as immortalized B-cells. The antibody can contain both light chain and heavy chain constant regions. The heavy chain constant region can include CH1, hinge, CH2, CH3, and, sometimes, CH4 regions. For therapeutic purposes, the CH2 domain can be deleted or omitted.

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody, antigen-binding portion thereof, or CAR as described herein. Such functional activities include stimulating PD1/PD-L1 interaction and/or anti-inflammatory activity.

Additionally, a polypeptide having functional activity means the polypeptide exhibits activity similar, but not necessarily identical to, an activity of a reference antibody, antigen-binding portion thereof, or CAR as described herein, including mature forms, as measured in a particular assay, such as, for example, a biological assay, with or without dose dependency. In the case where dose dependency does exist, it need not be identical to that of the reference antibody, antigen-binding portion thereof, or CAR, but rather substantially similar to the dose-dependence in a given activity as compared to the reference antibody, antigen-binding portion thereof, or CAR as described herein (i.e., the candidate polypeptide will exhibit greater activity, or not more than about 25-fold less, about 10-fold less, or about 3-fold less activity relative to the antibodies, antigen-binding portions, and/or CARs described herein).

In some embodiments, the antibody reagents (e.g., antibodies or CARs) described herein are not naturally-occurring biomolecules. For example, a murine antibody raised against an antigen of human origin would not occur in nature absent human intervention and manipulation, e.g., manufacturing steps carried out by a human. Chimeric antibodies are also not naturally-occurring biomolecules, e.g., in that they comprise sequences obtained from multiple species and assembled into a recombinant molecule. In certain particular embodiments, the human antibody reagents described herein are not naturally-occurring biomolecules, e.g., fully human antibodies directed against a human antigen would be subject to negative selection in nature and are not naturally found in the human body.

In some embodiments, the antibody, antibody reagent, antigen-binding portion thereof, and/or CAR is an isolated polypeptide. In some embodiments, the antibody, antibody reagent, antigen-binding portion thereof, and/or CAR is a purified polypeptide. In some embodiments, the antibody, antibody reagent, antigen-binding portion thereof, and/or CAR is an engineered polypeptide.

In one aspect of any of the embodiments, described herein is an antibody, antigen-binding fragment thereof, antigen reagent or chimaeric antigen receptor (CAR), that inhibits T-cell mediated responses. In some embodiments of any of the aspects, the antibody, antigen-binding fragment thereof, antigen reagent or CAR comprises at least one heavy or light chain complementarity determining region (CDR) selected from the group consisting of:
(a) a heavy chain CDR1 having the amino acid sequence of SEQ ID NO: 3;
(b) a heavy chain CDR2 having the amino acid sequence of SEQ ID NO: 4;
(c) a heavy chain CDR3 having the amino acid sequence of SEQ ID NO: 5;
(d) a light chain CDR1 having the amino acid sequence of SEQ ID NO: 6;
(e) a light chain CDR2 having the amino acid sequence of SEQ ID NO: 7; and
(f) a light chain CDR3 having the amino acid sequence of SEQ ID NO: 8, or
   selected from the group consisting of:
(a) a heavy chain CDR1 having the amino acid sequence of SEQ ID NO: 19;
(b) a heavy chain CDR2 having the amino acid sequence of SEQ ID NO: 20;
(c) a heavy chain CDR3 having the amino acid sequence of SEQ ID NO: 21;
(d) a light chain CDR1 having the amino acid sequence of SEQ ID NO: 22;
(e) a light chain CDR2 having the amino acid sequence of SEQ ID NO: 23; and
(f) a light chain CDR3 having the amino acid sequence of SEQ ID NO: 24; or
   a conservative substitution variant of one or more of (a)-(f).

In some embodiments of any of the aspects, the antibody, antibody reagent, antigen-binding portion thereof, or CAR includes heavy chain CDRs having the amino acid sequences of SEQ ID NOs: 3-5, or 19-21, or a conservative substitution variant of such amino acid sequence. In some embodiments of any of the aspects, the antibody, antibody reagent, antigen-binding portion thereof, or CAR includes light chain CDRs having the amino acid sequences of SEQ ID NOs: 6-8, or 22-24, or a conservative substitution variant of such amino acid sequence.

In some embodiments of any of the aspects, the antibody, antibody reagent, antigen-binding portion thereof, or CAR includes: heavy chain CDRs having the amino acid sequences of SEQ ID NOs: 3-5 and light chain CDRs having the amino acid sequences of SEQ ID NOs: 6-8 or a conservative substitution variant of such amino acid sequence; or heavy chain CDRs having the amino acid sequences of SEQ ID NOs: 19-21 and light chain CDRs having the amino acid sequences of SEQ ID NOs: 22-24 or a conservative substitution variant of such amino acid sequence.

In one aspect of any of the embodiments, described herein is a first antibody, antibody reagent, antigen-binding portion thereof, or CAR that specifically binds an PD1 polypeptide, and can compete for binding of PD1 with a second antibody comprising: heavy chain CDRs having the amino acid sequences of SEQ ID NOs: 3-5 and light chain CDRs having the amino acid sequences of SEQ ID NOs: 6-8 or a conservative substitution variant of such amino acid sequence. In some embodiments of any of the aspects, the first antibody, antibody reagent, antigen-binding fragment thereof, or chimeric antigen receptor (CAR) includes at least one heavy or light chain complementarity determining region (CDR) selected from the group consisting of:
(a) a heavy chain CDR1 having the amino acid sequence of SEQ ID NO: 3;
(b) a heavy chain CDR2 having the amino acid sequence of SEQ ID NO: 4;
(c) a heavy chain CDR3 having the amino acid sequence of SEQ ID NO: 5;
(d) a light chain CDR1 having the amino acid sequence of SEQ ID NO: 6;
(e) a light chain CDR2 having the amino acid sequence of SEQ ID NO: 7; and
(f) a light chain CDR3 having the amino acid sequence of SEQ ID NO: 8; or
   selected from the group consisting of:
(a) a heavy chain CDR1 having the amino acid sequence of SEQ ID NO: 19;
(b) a heavy chain CDR2 having the amino acid sequence of SEQ ID NO: 20;
(c) a heavy chain CDR3 having the amino acid sequence of SEQ ID NO: 21;
(d) a light chain CDR1 having the amino acid sequence of SEQ ID NO: 22;
(e) a light chain CDR2 having the amino acid sequence of SEQ ID NO: 23; and
(f) a light chain CDR3 having the amino acid sequence of SEQ ID NO: 24; or
   a conservative substitution variant of one or more of (a)-(f).

In alternative embodiments of any of the aspects and embodiments described herein, an antibody, antibody reagent, antigen-binding portion thereof which is described as comprising a heavy chain CDR2 having the amino acid sequence of SEQ ID NO: 4 alternatively comprises a heavy chain CDR2 having the amino acid sequence of LIWYDGSKKF (SEQ ID NO: 89).

In alternative embodiments of any of the aspects and embodiments described herein, an antibody, antibody reagent, antigen-binding portion thereof which is described as comprising a heavy chain CDR2 having the amino acid sequence of SEQ ID NO: 12 alternatively comprises a heavy chain CDR2 having the amino acid sequence of VIWYDGSRKH (SEQ ID NO: 90).

In alternative embodiments of any of the aspects and embodiments described herein, an antibody, antibody reagent, antigen-binding portion thereof which is described as comprising a heavy chain CDR2 having the amino acid sequence of SEQ ID NO: 28 alternatively comprises a heavy chain CDR2 having the amino acid sequence of VIWYDGSRKH (SEQ ID NO: 91).

In alternative embodiments of any of the aspects and embodiments described herein, an antibody, antibody reagent, antigen-binding portion thereof which is described as comprising a heavy chain CDR2 having the amino acid sequence of SEQ ID NO: 36 alternatively comprises a heavy chain CDR2 having the amino acid sequence of LIWYDGSKKF (SEQ ID NO: 92).

In alternative embodiments of any of the aspects and embodiments described herein, an antibody, antibody reagent, antigen-binding portion thereof which is described as comprising a heavy chain CDR2 having the amino acid sequence of SEQ ID NO: 44 alternatively comprises a heavy chain CDR2 having the amino acid sequence of VIWYDGGRKH (SEQ ID NO: 93).

In alternative embodiments of any of the aspects and embodiments described herein, an antibody, antibody reagent, antigen-binding portion thereof which is described as comprising a heavy chain CDR2 having the amino acid sequence of SEQ ID NO: 52 alternatively comprises a heavy chain CDR2 having the amino acid sequence of IIWYDGSRNH (SEQ ID NO: 94).

In alternative embodiments of any of the aspects and embodiments described herein, an antibody, antibody reagent, antigen-binding portion thereof which is described as comprising a heavy chain CDR2 having the amino acid sequence of SEQ ID NO: 60 alternatively comprises a heavy chain CDR2 having the amino acid sequence of VIWYDGSRKH (SEQ ID NO: 95).

In alternative embodiments of any of the aspects and embodiments described herein, an antibody, antibody reagent, antigen-binding portion thereof which is described as comprising a heavy chain CDR2 having the amino acid sequence of SEQ ID NO: 68 alternatively comprises a heavy chain CDR2 having the amino acid sequence of LIWYDGSKKY (SEQ ID NO: 96).

In alternative embodiments of any of the aspects and embodiments described herein, an antibody, antibody reagent, antigen-binding portion thereof which is described as comprising a heavy chain CDR2 having the amino acid sequence of SEQ ID NO: 76 alternatively comprises a heavy chain CDR2 having the amino acid sequence of LIWYDGTKKY (SEQ ID NO: 97).

In some embodiments of any of the aspects, the antibody, antibody reagent, antigen-binding portion thereof, or CAR includes the heavy chain variable region sequence of SEQ ID NO: 1 or 17. In some embodiments of any of the aspects, the antibody, antibody reagent, antigen-binding portion thereof, or CAR includes the light chain variable region sequence of SEQ ID NO: 2 or 18. In some embodiments of any of the aspects, the antibody, antibody reagent, antigen-binding portion thereof, or CAR includes: the heavy chain variable region sequence of SEQ ID NO: 1 and the light chain variable region sequence of SEQ ID NO: 2; or the heavy chain variable region sequence of SEQ ID NO: 17 and the light chain variable region sequence of SEQ ID NO: 18. In some embodiments of any of the aspects, the antibody, antibody reagent, antigen-binding portion thereof, or CAR further includes a conservative substitution in a sequence not comprised by a CDR, e.g., a conservative substitution relative to SEQ ID NO: 1 and/or 2, or a conservative substitution relative to SEQ ID NO: 17 and/or 18.

In some embodiments of any of the aspects, the antibody reagent or antigen-binding fragment thereof is fully human or fully humanized. In some embodiments of any of the aspects, the antibody reagent or antigen-binding fragment thereof is fully humanized except for the CDR sequences. In some embodiments of any of the aspects, the antibody reagent or antigen-binding fragment is selected from the group consisting of: an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, and a bispecific antibody.

In some embodiments, the antibody, antibody reagent, antigen-binding portion thereof, or CAR can comprise one or more CDRs (e.g., one CDR, two CDRs, three CDRs, four CDRs, five CDRs, or six CDRs) having the sequence of a CDR selected from SEQ ID NOs: 3-8, 11-16, and 19-24. In some embodiments, the antibody, antibody reagent, antigen-binding portion thereof, or CAR can comprise CDRs having the sequence of the CDRs of an antibody of Table 1.

In one aspect of any of the embodiments, described herein is an antibody, antibody reagent, antigen-binding portion thereof, or CAR that specifically binds an PD1 polypeptide, and can compete for binding of PD1 with an antibody selected from Table 1, or having the CDRs of an antibody selected from Table 1.

In some embodiments, the antibody, antibody reagent, antigen-binding portion thereof, and/or CAR as described herein can be a variant of a sequence described herein, e.g., a conservative substitution variant of an antibody polypeptide. In some embodiments, the variant is a conservatively modified variant. Conservative substitution variants can be obtained by mutations of native nucleotide sequences, for example.

A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or portion thereof that retains activity, e.g., antigen-specific binding activity for the relevant target polypeptide, e.g., PD1. A wide variety of PCR-based site-specific mutagenesis approaches are also known in the art and can be applied by the ordinarily skilled artisan.

One of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retain the ability to specifically bind the target antigen (e.g., PD1). Such conservatively modified variants are in addition to and do not exclude polymorphic variants, interspecies homologs, and alleles consistent with the disclosure.

Examples of substitution variants include conservative substitution of amino acids, e.g., in a V_{H} or V_{L}, domain, that do not alter the sequence of a CDR. A conservative substitution in a sequence not comprised by a CDR can be a substitution relative to a wild-type or naturally-occurring sequence, e.g., human or murine framework and/or constant regions of an antibody sequence. In some embodiments, a conservatively modified variant of an antibody reagent can comprise alterations other than in the CDRs, e.g., a conservatively modified variant of an antibody, antibody reagent, antigen-binding portion thereof, or CAR can comprise CDRs having the sequence of one or more of SEQ ID NOs 3-8, 11-16, and 19-24. In some embodiments, a conservatively modified variant of an antibody, antibody reagent, antigen-binding portion thereof, or CAR can comprise CDRs having the sequences of an antibody of Table 1.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g., antigen-binding activity and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

A variant amino acid or DNA sequence preferably is at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g., BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required.

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

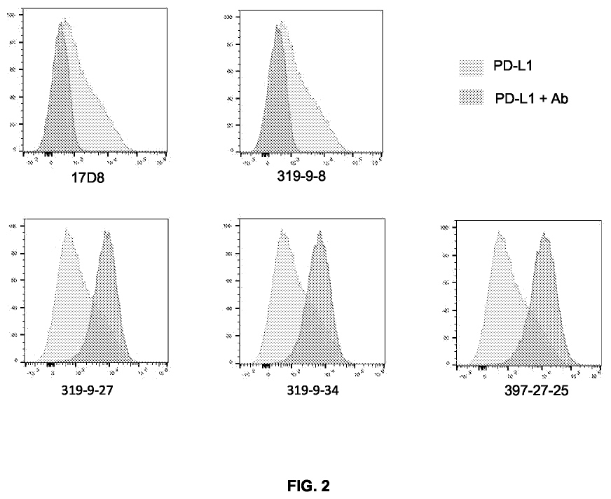

In particular embodiments wherein an antibody, antigen-binding portion thereof, or CAR as described herein comprises at least one CDR which is not identical to the sequence of SEQ ID NOs: 3-8, 11-16, and 19-24, the amino acid sequence of that at least one CDR can be selected by methods well known to one of skill in the art. For example, Fujii, 2004, “Antibody affinity maturation by random mutagenesis” in Methods in Molecular Biology: Antibody Engineering 248: 345-349 (incorporated by reference herein in its entirety), particularly at FIG. 2 and Section 3.3, describes methods of generating a library for any CDR of interest. This allows one of ordinary skill in the art to identify alternative CDRs, including conservative substitution variants of the specific CDR sequences described herein, which, when present in an antibody or antigen-binding portion thereof as described herein, will result in an antigen or antigen-binding portion thereof which will stimulate the PD1/PD-L1 interaction in cells. The method described in Fujii et al. also permits one of ordinary skill in the art to screen for a light chain sequence which will give the desired binding behavior when combined with a known heavy chain fragment and vice versa.

In some embodiments of any of the aspects, a conservative substitution variant has at least 80%, at least 85%, at least 90%, or at least 95% sequence identity to the applicable reference sequence (e.g., to one of the variable region sequences provided herein). In some embodiments of any of the aspects, a conservative substitution variant has at least 90% sequence identity to the applicable reference sequence (e.g., to one of the variable region sequences provided herein). In some embodiments of any of the aspects, a conservative substitution variant has at least 95% sequence identity to the applicable reference sequence (e.g., to one of the variable region sequences provided herein). In some embodiments of any of the aspects, a conservative substitution variant has at least 95% sequence identity to the applicable reference sequence (e.g., to one of the variable region sequences provided herein) and retains the wild-type activity of the reference sequence (e.g., the ability to bind specifically to PD1 and/or to agonize PD1).

In some embodiments, a CAR comprises an extracellular domain comprising an anti-PD1 antibody or antigen-binding portion thereof that binds one or more epitopes of a PD1 polypeptide; a transmembrane domain, one or more intracellular co-stimulatory signaling domains, and a primary signaling domain. Exemplary anti-PD1 antibodies and antigen-binding portions thereof, as well as exemplary epitopes, are described elsewhere herein.

As used herein, “chimeric antigen receptor” or “CAR” refers to an artificially constructed hybrid polypeptide comprising an antigen-binding domain (e.g., an antigen-binding portion of an antibody (e.g., a scFV)), a transmembrane domain, and a T-cell signaling and/or T-cell activation domain. CARs have the ability to redirect T-cell specificity and reactivity toward a selected target in a non-MHC-restricted manner, exploiting the antigen-binding properties of monoclonal antibodies. The non-MHC-restricted antigen recognition gives T-cells expressing CARs the ability to recognize an antigen independent of antigen processing, thus bypassing a major mechanism of tumor escape.

Moreover, when expressed in T-cells, CARs advantageously do not dimerize with endogenous T-cell receptor (TCR) alpha and beta chains. Most commonly, the CAR's extracellular binding domain is composed of a single chain variable fragment (scFv) derived from fusing the variable heavy and light regions of a murine or humanized monoclonal antibody. Alternatively, scFvs may be used that are derived from Fab's (instead of from an antibody, e.g., obtained from Fab libraries), in various embodiments, this scFv is fused to a transmembrane domain and then to an intracellular signaling domain.

“First-generation” CARs include those that solely provide CD3zeta (CD3ζ) signals upon antigen binding, “Second-generation” CARs include those that provide both costimulation (e.g., CD28 or CD 137) and activation (CD3ζ). “Third-generation” CARs include those that provide multiple costimulatory (e.g., CD28 and CD 137) domains and activation domains (e.g., CD3ζ). In various embodiments, the CAR is selected to have high affinity or avidity for the antigen. Further discussion of CARs can be found, e.g., in Maus et al. Blood 2014 123:2624-35; Reardon et al. Neuro-Oncology 2014 16:1441-1458; Hoyos et al. Haematologica 2012 97:1622; Byrd et al. J Clin Oncol 2014 32:3039-47; Maher et al. Cancer Res 2009 69:4559-4562; and Tamada et al. Clin Cancer Res 2012 18:6436-6445; each of which is incorporated by reference herein in its entirety.

In some embodiments of any of the aspects, a CAR comprises an extracellular binding domain that comprises a humanized PD1-specific binding domain; a transmembrane domain; one or more intracellular co-stimulatory signaling domains; and a primary signaling domain. As used herein, the terms, “binding domain,” “extracellular domain,” “extracellular binding domain,” “antigen-specific binding domain,” and “extracellular antigen specific binding domain,” are used interchangeably and provide a CAR with the ability to specifically bind to the target antigen of interest, e.g., PD1. The binding domain may be derived either from a natural, synthetic, semi-synthetic, or recombinant source.

In some embodiments, the CARs contemplated herein may comprise linker residues between the various domains, e.g., added for appropriate spacing and conformation of the molecule. In particular embodiments the linker is a variable region linking sequence. A “variable region linking sequence,” is an amino acid sequence that connects the VH and VL domains and provides a spacer function compatible with interaction of the two sub-binding domains so that the resulting polypeptide retains a specific binding affinity to the same target molecule as an antibody that comprises the same light and heavy chain variable regions. CARs contemplated herein, can comprise one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

In particular embodiments, the binding domain of the CAR is followed by one or more “spacer domains,” which refers to the region that moves the antigen binding domain away from the effector cell surface to enable proper cell/cell contact, antigen binding and activation. The hinge domain may be derived eitherfrom a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region.

The binding domain of the CAR is generally followed by one or more “hinge domains,” which plays a role in positioning the antigen binding domain away from the effector cell surface to enable proper cell/cell contact, antigen binding and activation. A CAR generally comprises one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region. Illustrative hinge domains suitable for use in the CARs described herein include the hinge region derived from the extracellular regions of type 1 membrane proteins such as CD8a, CD4, CD28 and CD7, which may be wild-type hinge regions from these molecules or may be altered. In another embodiment, the hinge domain comprises a CD8a hinge region.

The “transmembrane domain” is the portion of the CAR that fuses the extracellular binding portion and intracellular signaling domain and anchors the CAR to the plasma membrane of the immune effector cell. The TM domain may be derived either from a natural, synthetic, semi-synthetic, or recombinant source. The TM domain may be derived from (i.e., comprise at least the transmembrane region(s) of) the alpha, beta or zeta chain of the T-cell receptor, CD3ε, CD3ζ, CD4, CD5, CD8α, CD9, CD 16, CD22, CD27, CD28, CD33, CD37, CD45, CD64, CD80, CD86, CD 134, CD137, CD152, CD 154, and PD1.

In some embodiments, CARs contemplated herein comprise an intracellular signaling domain. An “intracellular signaling domain,” refers to the part of a CAR that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound targeT-cell, or other cellular responses elicited with antigen binding to the extracellular CAR domain. In some embodiments, a CAR contemplated herein comprises an intracellular signaling domain that comprises one or more “co-stimulatory signaling domain” and a “primary signaling domain.”

Primary signaling domains regulate primary activation of the TCR complex either in a stimulatory way, or in an inhibitory way. Primary signaling domains that act in a stimulatory manner may contain signaling motifs which are known as immunoreceptor tyrosine-based activation motifs or ITAMs. Illustrative examples of ITAM containing primary signaling domains that are of particular use in the invention include those derived from TCRζ, FcRγ, FcRβ, CD3γ, CD3δ, CD3ε, CD3ζ, CD22, CD79a, CD79b, and CD66d.

As used herein, the term, “co-stimulatory signaling domain,” or “co-stimulatory domain”, refers to an intracellular signaling domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory molecules include CARD11, CD2, CD7, CD27, CD28, CD30, CD40, CD54 (ICAM), CD83, CD134 (OX40), CD137 (4-1BB), CD150 (SLAMF1), CD152 (CTLA4), CD223 (LAG3), CD270 (HVEM), CD273 (PD-L2), CD274 (PD-L1), CD278 (ICOS), DAP10, LAT, NKD2C SLP76, TRIM, and ZAP70. In one embodiment, a CAR comprises one or more co-stimulatory signaling domains selected from the group consisting of CD28, CD137, and CD134, and a CD3ζ primary signaling domain.

In some embodiments, an antibody-drug conjugate is provided. In particular embodiments, an antibody-drug conjugate comprises an antibody, antibody reagent, or antigen-binding portion thereof as described herein. The drug can be, e.g., an immunosuppressive molecule as described elsewhere herein. In some embodiments, the antibody-drug conjugate comprises an immunosuppressive agent directly conjugated and/or bound to an antibody or antigen-binding portion thereof. In some embodiments, binding can be non-covalent, e.g., by hydrogen bonding, electrostatic, or van der Waals interactions; however, binding may also be covalent. By “conjugated” is meant the covalent linkage of at least two molecules. In some embodiments, the composition can be an antibody-drug conjugate.

In some embodiments, an antibody, antibody reagent, or antigen-binding portion thereof can be bound to and/or conjugated to multiple immunosuppressive molecules. In some embodiments, an antibody-drug conjugate can be bound to and/or conjugated to multiple immunosuppressive molecules. In some embodiments, the ratio of a given immunosuppressive molecule to an antibody or antigen-binding portion thereof can be from about 1:1 to about 1,000:1, e.g., a single antibody reagent molecule can be linked to, conjugated to, etc. from about 1 to about 1,000 individual immunosuppressive molecules.

In some embodiments, an antibody, or antigen-binding portion thereof, and the immunosuppressive agent can be present in a scaffold material. Scaffold materials suitable for use in therapeutic compositions are known in the art and can include, but are not limited to, a nanoparticle; a matrix; a hydrogel; and a biomaterial, biocompatible, and/or biodegradable scaffold material. As used herein, the term “nanoparticle” refers to particles that are on the order of about 10⁻⁹ or one to several billionths of a meter. The term “nanoparticle” includes nanospheres; nanorods; nanoshells; and nanoprisms; these nanoparticles may be part of a nanonetwork.

The term “nanoparticles” also encompasses liposomes and lipid particles having the size of a nanoparticle. As used herein, the term “matrix” refers to a 3-dimensional structure comprising the components of a composition described herein (e.g., an antibody or antigen-binding portion thereof). Non-limiting examples of matrix structures include foams; hydrogels; electrospun fibers; gels; fiber mats; sponges; 3-dimensional scaffolds; non-woven mats; woven materials; knit materials; fiber bundles; and fibers and other material formats (See, e.g., Rockwood et al. Nature Protocols 2011 6:1612-1631 and US Patent Publications 2011/0167602; 2011/0009960; 2012/0296352; and U.S. Pat. No. 8,172,901; each of which is incorporated by reference herein in its entirety). The structure of the matrix can be selected by one of skill in the art depending upon the intended application of the composition, e.g., electrospun matrices can have greater surface area than foams.

In some embodiments, the scaffold is a hydrogel. As used herein, the term “hydrogel” refers to a three-dimensional polymeric structure that is insoluble in water but which is capable of absorbing and retaining large quantities of water to form a stable, often soft and pliable, structure. In some embodiments, water can penetrate in between the polymer chains of the polymer network, subsequently causing swelling and the formation of a hydrogel. In general, hydrogels are superabsorbent. Hydrogels have many desirable properties for biomedical applications. For example, they can be made nontoxic and compatible with tissue, and they are highly permeable to water, ions, and small molecules. Hydrogels are super-absorbent (they can contain over 99% water) and can be comprised of natural (e.g., silk) or synthetic polymers, e.g., PEG.

As used herein, “biomaterial” refers to a material that is biocompatible and biodegradable. As used herein, the term “biocompatible” refers to substances that are not toxic to cells. In some embodiments, a substance is considered to be “biocompatible” if its addition to cells in vitro results in less than or equal to approximately 20% cell death. In some embodiments, a substance is considered to be “biocompatible” if its addition to cells in vivo does not induce inflammation and/or other adverse effects in vivo. As used herein, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

Provided herein are methods and compositions for the treatment of, e.g., inflammation, inflammatory conditions, and/or autoimmune diseases in a subject in need thereof. Provided herein are methods and compositions for the inhibition of an immune response or the inhibition of the immune system in a subject in need thereof. Such methods relate to administration of one or more of the antibodies, antibody reagent, antigen-binding portions thereof, or CARs to the subject. In some embodiments, the one or more antibodies, antibody reagent, antigen-binding portions thereof, or CARs comprise at least one heavy or light chain complementarity determining region (CDR) selected from the group consisting of:
(a) a heavy chain CDR1 having the amino acid sequence of SEQ ID NO: 3;
(b) a heavy chain CDR2 having the amino acid sequence of SEQ ID NO: 4;
(c) a heavy chain CDR3 having the amino acid sequence of SEQ ID NO: 5;
(d) a light chain CDR1 having the amino acid sequence of SEQ ID NO: 6;
(e) a light chain CDR2 having the amino acid sequence of SEQ ID NO: 7; and
(f) a light chain CDR3 having the amino acid sequence of SEQ ID NO: 8, or
   selected from the group consisting of:
(a) a heavy chain CDR1 having the amino acid sequence of SEQ ID NO: 11;
(b) a heavy chain CDR2 having the amino acid sequence of SEQ ID NO: 12;
(c) a heavy chain CDR3 having the amino acid sequence of SEQ ID NO: 13;
(d) a light chain CDR1 having the amino acid sequence of SEQ ID NO: 14;
(e) a light chain CDR2 having the amino acid sequence of SEQ ID NO: 15; and
(f) a light chain CDR3 having the amino acid sequence of SEQ ID NO: 16, or
   selected from the group consisting of:
(a) a heavy chain CDR1 having the amino acid sequence of SEQ ID NO: 19;
(b) a heavy chain CDR2 having the amino acid sequence of SEQ ID NO: 20;
(c) a heavy chain CDR3 having the amino acid sequence of SEQ ID NO: 21;
(d) a light chain CDR1 having the amino acid sequence of SEQ ID NO: 22;
(e) a light chain CDR2 having the amino acid sequence of SEQ ID NO: 23; and
(f) a light chain CDR3 having the amino acid sequence of SEQ ID NO: 24, or
   selected from the group consisting of:
(a) a heavy chain CDR1 having the amino acid sequence of SEQ ID NO: 27;
(b) a heavy chain CDR2 having the amino acid sequence of SEQ ID NO: 28;
(c) a heavy chain CDR3 having the amino acid sequence of SEQ ID NO: 29;
(d) a light chain CDR1 having the amino acid sequence of SEQ ID NO: 30;
(e) a light chain CDR2 having the amino acid sequence of SEQ ID NO: 31; and
(f) a light chain CDR3 having the amino acid sequence of SEQ ID NO: 32, or
   selected from the group consisting of:
(a) a heavy chain CDR1 having the amino acid sequence of SEQ ID NO: 35;
(b) a heavy chain CDR2 having the amino acid sequence of SEQ ID NO: 36;
(c) a heavy chain CDR3 having the amino acid sequence of SEQ ID NO: 37;
(d) a light chain CDR1 having the amino acid sequence of SEQ ID NO: 38;
(e) a light chain CDR2 having the amino acid sequence of SEQ ID NO: 39; and
(f) a light chain CDR3 having the amino acid sequence of SEQ ID NO: 40, or
   selected from the group consisting of:
(a) a heavy chain CDR1 having the amino acid sequence of SEQ ID NO: 43;
(b) a heavy chain CDR2 having the amino acid sequence of SEQ ID NO: 44;
(c) a heavy chain CDR3 having the amino acid sequence of SEQ ID NO: 45;
(d) a light chain CDR1 having the amino acid sequence of SEQ ID NO: 46;
(e) a light chain CDR2 having the amino acid sequence of SEQ ID NO: 47; and
(f) a light chain CDR3 having the amino acid sequence of SEQ ID NO: 48, or
   selected from the group consisting of:
(a) a heavy chain CDR1 having the amino acid sequence of SEQ ID NO: 51;
(b) a heavy chain CDR2 having the amino acid sequence of SEQ ID NO: 52;
(c) a heavy chain CDR3 having the amino acid sequence of SEQ ID NO: 53;
(d) a light chain CDR1 having the amino acid sequence of SEQ ID NO: 54;
(e) a light chain CDR2 having the amino acid sequence of SEQ ID NO: 55; and
(f) a light chain CDR3 having the amino acid sequence of SEQ ID NO: 56, or
   selected from the group consisting of:
(a) a heavy chain CDR1 having the amino acid sequence of SEQ ID NO: 59;
(b) a heavy chain CDR2 having the amino acid sequence of SEQ ID NO: 60;
(c) a heavy chain CDR3 having the amino acid sequence of SEQ ID NO: 61;
(d) a light chain CDR1 having the amino acid sequence of SEQ ID NO: 62;
(e) a light chain CDR2 having the amino acid sequence of SEQ ID NO: 63; and
(f) a light chain CDR3 having the amino acid sequence of SEQ ID NO: 64, or
   selected from the group consisting of:
(a) a heavy chain CDR1 having the amino acid sequence of SEQ ID NO: 67;
(b) a heavy chain CDR2 having the amino acid sequence of SEQ ID NO: 68;
(c) a heavy chain CDR3 having the amino acid sequence of SEQ ID NO: 69;
(d) a light chain CDR1 having the amino acid sequence of SEQ ID NO: 70;
(e) a light chain CDR2 having the amino acid sequence of SEQ ID NO: 71; and
(f) a light chain CDR3 having the amino acid sequence of SEQ ID NO: 72, or
   selected from the group consisting of:
(a) a heavy chain CDR1 having the amino acid sequence of SEQ ID NO: 75;
(b) a heavy chain CDR2 having the amino acid sequence of SEQ ID NO: 76;
(c) a heavy chain CDR3 having the amino acid sequence of SEQ ID NO: 77;
(d) a light chain CDR1 having the amino acid sequence of SEQ ID NO: 78;
(e) a light chain CDR2 having the amino acid sequence of SEQ ID NO: 79; and
(f) a light chain CDR3 having the amino acid sequence of SEQ ID NO: 80, or
   a conservative substitution variant of one or more of (a)-(f).

As used herein, “inflammation” refers to the complex biological response to harmful stimuli, such as pathogens, damaged cells, or irritants. Inflammation is a protective attempt by the organism to remove the injurious stimuli as well as initiate the healing process for the tissue. Accordingly, the term “inflammation” includes any cellular process that leads to the production of pro-inflammatory cytokines, inflammation mediators and/or the related downstream cellular events resulting from the actions of the cytokines thus produced, for example, fever, fluid accumulation, swelling, abscess formation, and cell death. Inflammation can include both acute responses (i.e., responses in which the inflammatory processes are active) and chronic responses (i.e., responses marked by slow progression and formation of new connective tissue). Acute and chronic inflammation may be distinguished by the cell types involved. Acute inflammation often involves polymorphonuclear neutrophils; whereas chronic inflammation is normally characterized by a lymphohistiocytic and/or granulomatous response.

An inflammatory condition is any disease state characterized by inflammatory tissues (for example, infiltrates of leukocytes such as lymphocytes, neutrophils, macrophages, eosinophils, mast cells, basophils and dendritic cells) or inflammatory processes which provoke or contribute to the abnormal clinical and histological characteristics of the disease state. Inflammatory conditions include, but are not limited to, inflammatory conditions of the skin, inflammatory conditions of the lung, inflammatory conditions of the joints, inflammatory conditions of the gut, inflammatory conditions of the eye, inflammatory conditions of the endocrine system, inflammatory conditions of the cardiovascular system, inflammatory conditions of the kidneys, inflammatory conditions of the liver, inflammatory conditions of the central nervous system, or sepsis-associated conditions. In some embodiments, the inflammatory condition is associated with wound healing. In some embodiments, the inflammation to be treated according to the methods described herein can be skin inflammation; inflammation caused by substance abuse or drug addiction; inflammation associated with infection; inflammation of the cornea; inflammation of the retina; inflammation of the spinal cord; inflammation associated with organ regeneration; and pulmonary inflammation.

In some embodiments, an inflammatory condition can be an autoimmune disease. Non-limiting examples of autoimmune diseases can include: Type 1 diabetes; systemic lupus erythematosus; rheumatoid arthritis; psoriasis; inflammatory bowel disease; Crohn's disease; and autoimmune thyroiditis.

In some embodiments, a subject in need of treatment for inflammation can be a subject having, or diagnosed as having temporomandibular joint disorders; COPD; smoke-induced lung injury; renal dialysis associated disorders; spinal cord injury; graft vs. host disease; bone marrow transplant or complications thereof, infection; trauma; pain; incisions; surgical incisions; a chronic pain disorder; a chronic bone disorder; mastitis; and joint disease. In some embodiments, trauma can include battle-related injuries or tissue damage occurring during a surgery. Smoke-induced lung injury can result from exposure to tobacco smoke, environmental pollutants (e.g. smog or forest fires), or industrial exposure.

By way of non-limiting example, inflammatory conditions can be inflammatory conditions of the skin, such as Sweet's syndrome, pyoderma gangrenosum, subcorneal pustular dermatosis, erythema elevatum diutinum, Behcet's disease or acute generalized exanthematous pustulosis, a bullous disorder, psoriasis, a condition resulting in pustular lesions, acne, acne vulgaris, dermatitis (e.g. contact dermatitis, atopic dermatitis, seborrheic dermatitis, eczematous dermatitides, eczema craquelee, photoallergic dermatitis, phototoxicdermatitis, phytophotodermatitis, radiation dermatitis, stasis dermatitis or allergic contact dermatitis), eczema, ulcers and erosions resulting from trauma, burns, ischemia of the skin or mucous membranes, several forms of ichthyoses, epidermolysis bullosae, hypertrophic scars, keloids, cutaneous changes of intrinsic aging, photoaging, frictional blistering caused by mechanical shearing of the skin, cutaneous atrophy resulting from the topical use of corticosteroids, and inflammation of mucous membranes (e.g., cheilitis, chapped lips, nasal irritation, mucositis and vulvovaginitis).

By way of non-limiting example, inflammatory conditions can be inflammatory conditions of the lung, such as asthma, bronchitis, chronic bronchitis, bronchiolitis, pneumonia, sinusitis, emphysema, adult respiratory distress syndrome, pulmonary inflammation, pulmonary fibrosis, and cystic fibrosis (which may additionally or alternatively involve the gastro-intestinal tract or other tissue(s)). By way of non-limiting example, inflammatory conditions can be inflammatory conditions of the joints, such as rheumatoid arthritis, rheumatoid spondylitis, juvenile rheumatoid arthritis, osteoarthritis, gouty arthritis, infectious arthritis, psoriatic arthritis, and other arthritic conditions.

By way of non-limiting example, inflammatory conditions can be inflammatory conditions of the gut or bowel, such as inflammatory bowel disease, Crohn's disease, ulcerative colitis and distal proctitis. By way of non-limiting example, inflammatory conditions can be inflammatory conditions of the eye, such as dry eye syndrome, uveitis (including iritis), conjunctivitis, scleritis, and keratoconjunctivitis sicca. By way of non-limiting example, inflammatory conditions can be inflammatory conditions of the endocrine system, such as autoimmune thyroiditis (Hashimoto's disease), Graves' disease, Type I diabetes, and acute and chronic inflammation of the adrenal cortex.

By way of non-limiting example, inflammatory conditions can be inflammatory conditions of the cardiovascular system, such as coronary infarct damage, peripheral vascular disease, myocarditis, vasculitis, revascularization of stenosis, artherosclerosis, and vascular disease associated with Type II diabetes. By way of non-limiting example, inflammatory conditions can be inflammatory conditions of the kidneys, such as glomerulonephritis, interstitial nephritis, lupus nephritis, and nephritis secondary to Wegener's disease, acute renal failure secondary to acute nephritis, post-obstructive syndrome and tubular ischemia.

By way of non-limiting example, inflammatory conditions can be inflammatory conditions of the liver, such as hepatitis (arising from viral infection, autoimmune responses, drug treatments, toxins, environmental agents, or as a secondary consequence of a primary disorder), biliary atresia, primary biliary cirrhosis and primary sclerosing cholangitis. By way of non-limiting example, inflammatory conditions can be inflammatory conditions of the central nervous system, such as multiple sclerosis and neurodegenerative diseases such as Alzheimer's disease or dementia associated with HIV infection.

By way of non-limiting example, inflammatory conditions can be inflammatory conditions of the central nervous system, such as MS; all types of encephalitis and meningitis; acute disseminated encephalomyelitis; acute transverse myelitis; neuromyelitis optica; focal demyelinating syndromes (e.g., Balo's concentric sclerosis and Marburg variant of MS); progressive multifocal leukoencephalopathy; subacute sclerosing panencephalitis; acute haemorrhagic leucoencephalitis (Hurst's disease); human T-lymphotropic virus type-Iassociated myelopathy/tropical spactic paraparesis; Devic's disease; human immunodeficiency virus encephalopathy; human immunodeficiency virus vacuolar myelopathy; peripheral neuropathies; Guillain-Barre Syndrome and other immune mediated neuropathies; and myasthenia gravis. By way of non-limiting example, inflammatory conditions can be sepsis-associated conditions, such as systemic inflammatory response syndrome (SIRS), septic shock or multiple organ dysfunction syndrome (MODS).

Further non-limiting examples of inflammatory conditions include, endotoxin shock, periodontal disease, polychondritis; periarticular disorders; pancreatitis; system lupus erythematosus; Sjogren's syndrome; vasculitis sarcoidosis amyloidosis; allergies; anaphylaxis; systemic mastocytosis; pelvic inflammatory disease; multiple sclerosis; multiple sclerosis (MS); celiac disease, Guillain-Barre syndrome, sclerosing cholangitis, autoimmune hepatitis, Raynaud's phenomenon, Goodpasture's syndrome, Wegener's granulomatosis, polymyalgia rheumatica, temporal arteritis/gianT-cell arteritis, chronic fatigue syndrome CFS), autoimmune Addison's Disease, ankylosing spondylitis, Acute disseminated encephalomyelitis, antiphospholipid antibody syndrome, aplastic anemia, idiopathic thrombocytopenic purpura, Myasthenia gravis, opsoclonus myoclonus syndrome, optic neuritis, Ord's thyroiditis, pemphigus, pernicious anaemia, polyarthritis in dogs, Reiter's syndrome, Takayasu's arteritis, warm autoimmune hemolytic anemia, fibromyalgia (FM), autoinflammatory PAPA syndrome, Familial Mediaterranean Fever, polymyalgia rheumatica, polyarteritis nodosa, churg strauss syndrome; fibrosing alveolitis, hypersensitivity pneumonitis, allergic aspergillosis, cryptogenic pulmonary eosinophilia, bronchiolitis obliterans organising pneumonia; urticaria; lupoid hepatitis; familial cold autoinflammatory syndrome, Muckle-Wells syndrome, the neonatal onset multisystem inflammatory disease, graft rejection (including allograft rejection and graft-v-host disease), otitis, chronic obstructive pulmonary disease, sinusitis, chronic prostatitis, reperfusion injury, silicosis, inflammatory myopathies, hypersensitivities and migraines.

In some embodiments, an inflammatory condition is associated with an infection, e.g., viral, bacterial, fungal, parasite or prion infections. In some embodiments, an inflammatory condition is associated with an allergic response. In some embodiments, an inflammatory condition is associated with a pollutant (e.g., asbestosis, silicosis, or berylliosis).

By way of non-limiting example, if a subject is to be treated for inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g., endorphins, enkephalins and dynorphin), steroids, analgesics, barbiturates, oxycodone, morphine, lidocaine and the like.

In some embodiments, the methods described herein relate to the treatment or prevention of an autoimmune disease with compositions, CARs, or cells as described herein. Subjects having an autoimmune disease can be identified by a physician using current methods of diagnosing an autoimmune disease. Symptoms and/or complications of an autoimmune disease which characterize these conditions and aid in diagnosis are well known in the art and include but are not limited to, fatigue, achy muscles, swelling and redness, low-grade fever, numbness or tingling of the hands or feet, hair loss, and/or skin rash.

Tests that may aid in a diagnosis of, e.g. autoimmune disease include, but are not limited to, blood counts, and an antinuclear antibody test (ANA). A family history of autoimmune disease, or having risk factors for autoimmune disease (e.g. gender, age, ethnicity, and exposure to environmental agents, such as procainamide, hydrolyzine, mercury, gold, or silver) can also aid in determining if a subject is likely to have autoimmune disease or in making a diagnosis of autoimmune disease.

As used herein, the term “autoimmune disease” or “autoimmune disease or disorder” herein is a disease or disorder arising from and directed against an individual's own tissues or a co-segregate or manifestation thereof or resulting condition therefrom.

Auto-immune related diseases and disorders arise from an overactive and/or abnormal immune response of the body against substances (autoantigens) and tissues normally present in the body, otherwise known as self or autologous substance. This dysregulated inflammatory reaction causes an exaggerated response by macrophages, granulocytes, and/or T-lymphocytes leading to abnormal tissue damage and cell death. Subsequent loss of function is associated with inflammatory tissue damage.

Autoantigens, as used herein, are endogenous proteins or fragments thereof that elicit this pathogenic immune response. Autoantigen can be any substance or a portion thereof normally found within a mammal that, in an autoimmune disease, becomes the primary (or a primary) target of attack by the immune system. The term also includes antigenic substances that induce conditions having the characteristics of an autoimmune disease when administered to mammals.

Additionally, the term includes peptic subclasses consisting essentially of immunodominant epitopes or immunodominant epitope regions of autoantigens. Immunodominant epitopes or regions in induced autoimmune conditions are fragments of an autoantigen that can be used instead of the entire autoantigen to induce the disease. In humans afflicted with an autoimmune disease, immunodominant epitopes or regions are fragments of antigens specific to the tissue or organ under autoimmune attack and recognized by a substantial percentage (e.g. a majority though not necessarily an absolute majority) of autoimmune attack T-cells.

Autoantigens that are known to be associated with autoimmune disease include myelin proteins with demyelinating diseases, e.g. multiple sclerosis and experimental autoimmune myelitis; collagens and rheumatoid arthritis; insulin, proinsulin, glutamic acid decarboxylase 65 (GAD65); isleT-cell antigen (ICA512; ICA12) with insulin dependent diabetes.

A common feature in a number of autoimmune related diseases and inflammatory conditions is the involvement of pro-inflammatory CD4+ T-cells. These T-cells are responsible for the release of inflammatory, Th1 type cytokines. Cytokines characterized as Th1 type include interleukin 2 (IL-2), γ-interferon, TNFα and IL-12. Such pro-inflammatory cytokines act to stimulate the immune response, in many cases resulting in the destruction of autologous tissue. Cytokines associated with suppression of T-cell response are the Th2 type, and include IL-10, IL-4 and TGF-β. It has been found that Th1 and Th2 type T-cells may use the identical antigen receptor in response to an immunogen; in the former producing a stimulatory response and in the latter a suppressive response.

In one embodiment of any one of the method described, the autoimmune disorder is selected from the group consisting of thyroiditis, type 1 diabetes mellitus, Hashimoto's thyroidits, Graves' disease, celiac disease, multiple sclerolsis, Guillain-Barre syndrome, Addison's disease, and Raynaud's phenomenon, Goodpasture's disease, arthritis (rheumatoid arthritis such as acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, and juvenile-onset rheumatoid arthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, atopy including atopic diseases such as hay fever and Job's syndrome, dermatitis including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis, x-linked hyper IgM syndrome, allergic intraocular inflammatory diseases, urticaria such as chronic allergic urticaria and chronic idiopathic urticaria, including chronic autoimmune urticaria, myositis, polymyositis/dermatomyositis, juvenile dermatomyositis, toxic epidermal necrolysis, scleroderma (including systemic scleroderma), sclerosis such as systemic sclerosis, multiple sclerosis (MS) such as spino-optical MS, primary progressive MS (PPMS), and relapsing remitting MS (RRMS), progressive systemic sclerosis, atherosclerosis, arteriosclerosis, sclerosis disseminata, ataxic sclerosis, neuromyelitis optica (NMO), inflammatory bowel disease (IBD) (for example, Crohn's disease, autoimmune-mediated gastrointestinal diseases, colitis such as ulcerative colitis, colitis ulcerosa, microscopic colitis, collagenous colitis, colitis polyposa, necrotizing enterocolitis, and transmural colitis, and autoimmune inflammatory bowel disease), bowel inflammation, pyoderma gangrenosum, erythema nodosum, primary sclerosing cholangitis, respiratory distress syndrome, including adult or acute respiratory distress syndrome (ARDS), meningitis, inflammation of all or part of the uvea, iritis, choroiditis, an autoimmune hematological disorder, rheumatoid spondylitis, rheumatoid synovitis, hereditary angioedema, cranial nerve damage as in meningitis, herpes gestationis, pemphigoid gestationis, pruritis scroti, autoimmune premature ovarian failure, sudden hearing loss due to an autoimmune condition, IgE-mediated diseases such as anaphylaxis and allergic and atopic rhinitis, encephalitis such as Rasmussen's encephalitis and limbic and/or brainstem encephalitis, uveitis, such as anterior uveitis, acute anterior uveitis, granulomatous uveitis, nongranulomatous uveitis, phacoantigenic uveitis, posterior uveitis, or autoimmune uveitis, glomerulonephritis (GN) with and without nephrotic syndrome such as chronic or acute glomerulonephritis such as primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN, proliferative nephritis, autoimmune polyglandular endocrine failure, balanitis including balanitis circumscripta plasmacellularis, balanoposthitis, erythema annulare centrifugum, erythema dyschromicum perstans, eythema multiform, granuloma annulare, lichen nitidus, lichen sclerosus et atrophicus, lichen simplex chronicus, lichen spinulosus, lichen planus, lamellar ichthyosis, epidermolytic hyperkeratosis, premalignant keratosis, pyoderma gangrenosum, allergic conditions and responses, allergic reaction, eczema including allergic or atopic eczema, asteatotic eczema, dyshidrotic eczema, and vesicular palmoplantar eczema, asthma such as asthma bronchiale, bronchial asthma, and auto-immune asthma, conditions involving infiltration of T-cells and chronic inflammatory responses, immune reactions against foreign antigens such as fetal A-B-O blood groups during pregnancy, chronic pulmonary inflammatory disease, autoimmune myocarditis, leukocyte adhesion deficiency, lupus, including lupus nephritis, lupus cerebritis, pediatric lupus, non-renal lupus, extra-renal lupus, discoid lupus and discoid lupus erythematosus, alopecia lupus, systemic lupus erythematosus (SLE) such as cutaneous SLE or subacute cutaneous SLE, neonatal lupus syndrome (NLE), and lupus erythematosus disseminatus, juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, diabetic large-artery disorder, immune responses associated with acute and delayed hypersensitivity mediated by cytokines and T-lymphocytes, sarcoidosis, granulomatosis including lymphomatoid granulomatosis, Wegener's granulomatosis, agranulocytosis, vasculitides, including vasculitis, large-vessel vasculitis (including polymyalgia rheumatica and giant-cell (Takayasu's) arteritis), medium-vessel vasculitis (including Kawasaki's disease and polyarteritis nodosa/periarteritis nodosa), microscopic polyarteritis, immunovasculitis, CNS vasculitis, cutaneous vasculitis, hypersensitivity vasculitis, necrotizing vasculitis such as systemic necrotizing vasculitis, and ANCA-associated vasculitis, such as Churg-Strauss vasculitis or syndrome (CSS) and ANCA-associated small-vessel vasculitis, temporal arteritis, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Behcet's disease/syndrome, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus (including pemphigus vulgaris, pemphigus foliaceus, pemphigus mucus-membrane pemphigoid, and pemphigus erythematosus), autoimmune polyendocrinopathies, Reiter's disease or syndrome, an immune complex disorder such as immune complex nephritis, antibody-mediated nephritis, polyneuropathies, chronic neuropathy such as IgM polyneuropathies or IgM-mediated neuropathy, and autoimmune or immune-mediated thrombocytopenia such as idiopathic thrombocytopenic purpura (ITP) including chronic or acute ITP, scleritis such as idiopathic cerato-scleritis, episcleritis, autoimmune disease of the testis and ovary including autoimmune orchitis and oophoritis, primary hypothyroidism, hypoparathyroidism, autoimmune endocrine diseases including thyroiditis such as autoimmune thyroiditis, Hashimoto's disease, chronic thyroiditis (Hashimoto's thyroiditis), or subacute thyroiditis, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis such as allergic encephalomyelitis or encephalomyelitis allergica and experimental allergic encephalomyelitis (EAE), myasthenia gravis such as thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, lupoid hepatitis, giant-cell hepatitis, autoimmune chronic active hepatitis, lymphoid interstitial pneumonitis (LIP), bronchiolitis obliterans (non-transplant) vs NSIP, Guillain-Barre syndrome, Berger's disease (IgA nephropathy), idiopathic IgA nephropathy, linear IgA dermatosis, acute febrile neutrophilic dermatosis, subcorneal pustular dermatosis, transient acantholytic dermatosis, cirrhosis such as primary biliary cirrhosis and pneumonocirrhosis, autoimmune enteropathy syndrome, Celiac or Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AIED), autoimmune hearing loss, polychondritis such as refractory or relapsed or relapsing polychondritis, pulmonary alveolar proteinosis, Cogan's syndrome/nonsyphilitic interstitial keratitis, Bell's palsy, Sweet's disease/syndrome, rosacea autoimmune, zoster-associated pain, amyloidosis, a non-cancerous lymphocytosis, a primary lymphocytosis, which includes monoclonal B-cell lymphocytosis (e.g., benign monoclonal gammopathy and monoclonal gammopathy of undetermined significance, MGUS), peripheral neuropathy, paraneoplastic syndrome, channelopathies including channelopathies of the CNS, autism, inflammatory myopathy, focal or segmental or focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases and chronic inflammatory demyelinating polyneuropathy, Dressler's syndrome, alopecia areata, alopecia totalis, CREST syndrome (calcinosis, Raynaud's phenomenon, esophageal dysmotility, sclerodactyly, and telangiectasia), male and female autoimmune infertility, e.g., due to anti-spermatozoan antibodies, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, Sampter's syndrome, Caplan's syndrome, endocarditis, endomyocardial fibrosis, diffuse interstitial pulmonary fibrosis, interstitial lung fibrosis, pulmonary fibrosis, idiopathic pulmonary fibrosis, cystic fibrosis, endophthalmitis, erythema elevatum et diutinum, erythroblastosis fetalis, eosinophilic faciitis, Shulman's syndrome, Felty's syndrome, cyclitis such as chronic cyclitis, heterochronic cyclitis, iridocyclitis (acute or chronic), or Fuch's cyclitis, Henoch-Schonlein purpura, SCID, sepsis, endotoxemia, post-vaccination syndromes, Evan's syndrome, autoimmune gonadal failure, Sydenham's chorea, post-streptococcal nephritis, thromboangitis ubiterans, thyrotoxicosis, tabes dorsalis, chorioiditis, giant-cell polymyalgia, chronic hypersensitivity pneumonitis, keratoconjunctivitis sicca, idiopathic nephritic syndrome, minimal change nephropathy, benign familial and ischemia-reperfusion injury, transplant organ reperfusion, retinal autoimmunity, aphthae, aphthous stomatitis, arteriosclerotic disorders, aspermiogenesis, autoimmune hemolysis, Boeck's disease, enteritis allergica, erythema nodosum leprosum, idiopathic facial paralysis, chronic fatigue syndrome, febris rheumatica, Hamman-Rich's disease, sensoneural hearing loss, ileitis regionalis, leucopenia, transverse myelitis, primary idiopathic myxedema, ophthalmia symphatica, polyradiculitis acuta, pyoderma gangrenosum, acquired spenic atrophy, vitiligo, toxic-shock syndrome, conditions involving infiltration of T-cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, rheumatic diseases, mixed connective tissue disease, nephrotic syndrome, insulitis, polyendocrine failure, autoimmune polyglandular syndrome type I, adult-onset idiopathic hypoparathyroidism (AOIH), myocarditis, nephrotic syndrome, primary sclerosing cholangitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, granulomas containing eosinophils, seronegative spondyloarthritides, polyendocrine autoimmune disease, sclerosing cholangitis, sclera, episclera, Bruton's syndrome, transient hypogammaglobulinemia of infancy, Wiskott-Aldrich syndrome, ataxia telangiectasia syndrome, angiectasis, autoimmune disorders associated with collagen disease, rheumatism, allergic hypersensitivity disorders, glomerulonephritides, reperfusion injury, ischemic re-perfusion disorder, lymphomatous tracheobronchitis, inflammatory dermatoses, dermatoses with acute inflammatory components, and autoimmune uveoretinitis (AUR).

The compositions and methods described herein can be administered to a subject to treat or prevent an autoimmune disease or transplant rejection. In some embodiments, the methods described herein comprise administering an effective amount of compositions, CARs, or cells described herein to a subject in order to alleviate a symptom of an autoimmune disease or transplant rejection.

As used herein, “alleviating a symptom” is ameliorating any condition or symptom associated with the disease. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, or injection administration. Administration can be local or systemic.

In some embodiments of any of the aspects, described herein is a method of decreasing or suppressing an immune response in a subject in need thereof, the method comprising administering to the subject an antibody, antibody reagent, antigen-binding portion thereof, CAR, composition, or cell as described herein. In some embodiments of any of the aspects, the immune response is localized.

As used herein, an “immune response” refers to a response by a cell of the immune system, such as a B-cell, T-cell (CD4 or CD8), regulatory T-cell, antigen-presenting cell, dendritic cell, monocyte, macrophage, NKT-cell, NK cell, basophil, eosinophil, or neutrophil, to a stimulus (e.g., to a disease, an antigen, or healthy cells, e.g., in the case of autoimmunity). In some embodiments of the aspects described herein, an immune response is a T-cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. Stimulation of an immune response refers to an induction or increase of the immune response. Suppression of an immune response refers to an elimination or decrease of the immune response.

An immune response to an antigen can be the development in a subject of a humoral and/or a cell-mediated immune response to molecules present in the antigen or vaccine composition of interest. For purposes of the present invention, a “humoral immune response” is an antibody-mediated immune response and involves the induction and generation of antibodies that recognize and bind with some affinity for the antigen, while a “cell-mediated immune response” is one mediated by T-cells and/or other white blood cells.

A “cell-mediated immune response” is elicited by the presentation of antigenic epitopes in association with Class I or Class II molecules of the major histocompatibility complex (MHC), CD1 or other non-classical MHC-like molecules. This activates antigen-specific CD4+ T helper cells or CD8+ cytotoxic lymphocyte cells (“CTLs”). CTLs have specificity for peptide antigens that are presented in association with proteins encoded by classical or non-classical MHCs and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide or other antigens in association with classical or non-classical MHC molecules on their surface.

A “cell-mediated immune response” also refers to the production of cytokines, chemokines and other such molecules produced by activated T-cells and/or other white blood cells, including those derived from CD4+ and CD8+ T-cells. The ability of a particular antigen or composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T-cells in response to re-stimulation with antigen. Such assays are well known in the art. See, e.g., Erickson et al. (1993) J. Immunol. 151:4189-4199; and Doe et al. (1994) Eur. J. Immunol. 24:2369-2376.

In some embodiments, the technology described herein relates to a nucleic acid encoding an antibody, antibody reagent, antigen-binding portion thereof, or CAR as described herein. In some embodiments, the nucleic acid is a cDNA.

As used herein, the term “nucleic acid” or “nucleic acid sequence” refers to a polymeric molecule incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. In some embodiments, the nucleic acid can be a cDNA, e.g., a nucleic acid lacking introns.

Nucleic acid molecules encoding amino acid sequence variants of antibodies are prepared by a variety of methods known in the art. These methods include, but are not limited to preparation by oligonucleotide-mediated (or site-directed) mutagenesis, PCR mutagenesis, and cassette mutagenesis of an earlier prepared variant or a non-variant version of the antibody. A nucleic acid sequence encoding at least one antibody, portion or polypeptide as described herein can be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations can be used to construct nucleic acid sequences which encode a monoclonal antibody molecule, antibody reagent, antigen binding region thereof, or CAR.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene expression as peptides or antibody portions in recoverable amounts. The precise nature of the regulatory regions needed for gene expression may vary from organism to organism, as is well known in the analogous art.

In some embodiments, a nucleic acid encoding an antibody, antibody reagent, antigen-binding portion thereof, or CAR as described herein is comprised by a vector. In some of the aspects described herein, a nucleic acid sequence encoding an antibody, antibody reagent, antigen-binding portion thereof, or CAR as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding an antibody, antigen-binding portion thereof, or CAR as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

In one aspect of any of the embodiments, described herein is a cell comprising an antibody, antibody reagent, antigen-binding portion thereof, or CAR as described herein, or a nucleic acid encoding such an antibody, antibody reagent, antigen-binding portion thereof, or CAR.

The expression of an antibody, antibody reagent, antigen-binding portion thereof, or CAR as described herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of an antibody or portion thereof as described herein with a specified amino terminus sequence.

Moreover, problems associated with retention of initiation codon-derived methionine residues in direct yeast (or bacterial) expression maybe avoided. Any of a series of yeast gene expression systems incorporating promoter and termination elements from the actively expressed genes coding for glycolytic enzymes produced in large quantities when yeast is grown in mediums rich in glucose can be utilized to obtain recombinant antibodies or antigen-binding portions thereof as described herein. Known glycolytic genes can also provide very efficient transcriptional control signals. For example, the promoter and terminator signals of the phosphoglycerate kinase gene can be utilized.

Production of antibodies or antigen-binding portions thereof as described herein in insects can be achieved. For example, by infecting the insect host with a baculovirus engineered to express a transmembrane polypeptide by methods known to those of ordinary skill in the art.

In some embodiments, the introduced nucleotide sequence is incorporated into a plasmid or viral vector capable of autonomous replication in the recipient host. Any of a wide variety of vectors can be employed for this purpose and are known and available to those or ordinary skill in the art. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Examplary prokaryotic vectors known in the art include plasmids such as those capable of replication in E. coli., for example. Other gene expression elements useful for the expression of cDNA encoding antibodies, antigen-binding portions thereof, or CARs include, but are not limited to (a) viral transcription promoters and their enhancer elements, such as the SV40 early promoter, Rous sarcoma virus LTR, and Moloney murine leukemia virus; (b) splice regions and polyadenylation sites such as those derived from the SV40 late region, and (c) polyadenylation sites such as in SV40. Immunoglobulin cDNA genes can be expressed, e.g., using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin genes comprised of part cDNA, part genomic DNA, the transcriptional promoter can be human cytomegalovirus, the promoter enhancers can be cytomegalovirus and mouse/human immunoglobulin, and mRNA splicing and polyadenylation regions can be the native chromosomal immunoglobulin sequences.

In some embodiments, for expression of cDNA genes in rodent cells, the transcriptional promoter is a viral LTR sequence, the transcriptional promoter enhancers are either or both the mouse immunoglobulin heavy chain enhancer and the viral LTR enhancer, the splice region contains an intron of greater than 31 bp, and the polyadenylation and transcription termination regions are derived from the native chromosomal sequence corresponding to the immunoglobulin chain being synthesized. In other embodiments, cDNA sequences encoding other proteins are combined with the above-recited expression elements to achieve expression of the proteins in mammalian cells.

A gene is assembled in, or inserted into, an expression vector. Recipient cells capable of expressing the chimeric immunoglobulin chain gene product are then transfected singly with an antibody, antigen-binding portion thereof, or CAR, or chimeric H or chimeric L chain-encoding gene, or are co-transfected with a chimeric H and a chimeric L chain gene. The transfected recipient cells are cultured under conditions that permit expression of the incorporated genes and the expressed immunoglobulin chains or intact antibodies or fragments are recovered from the culture.

In some embodiments, the genes encoding the antibody, antigen-binding portion thereof, CAR, or chimeric H and L chains, or portions thereof are assembled in separate expression vectors that are then used to co-transfect a recipienT-cell. Each vector can contain two selectable genes, a first selectable gene designed for selection in a bacterial system and a second selectable gene designed for selection in a eukaryotic system, wherein each vector has a different pair of genes. This strategy results in vectors which first direct the production, and permit amplification, of the genes in a bacterial system. The genes so produced and amplified in a bacterial host are subsequently used to co-transfect a eukaryotic cell, and allow selection of a co-transfected cell carrying the desired transfected genes.

Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively, the genes can be assembled on the same expression vector.

For transfection of the expression vectors and production of the antibodies, antibody reagents, antigen-binding portions thereof, or CARs described herein, the recipienT-cell line can be a myeloma cell. Myeloma cells can synthesize, assemble and secrete immunoglobulins encoded by transfected immunoglobulin genes and possess the mechanism for glycosylation of the immunoglobulin. For example, in some embodiments, the recipienT-cell is the recombinant Ig-producing myeloma cell SP2/0 (ATCC #CRL 8287). SP2/0 cells produce only immunoglobulin encoded by the transfected genes. Myeloma cells can be grown in culture or in the peritoneal cavity of a mouse, where secreted immunoglobulin can be obtained from ascites fluid. Other suitable recipient cells include lymphoid cells such as B lymphocytes of human or non-human origin, hybridoma cells of human or non-human origin, or interspecies heterohybridoma cells.

An expression vector carrying a chimeric, humanized, or composite human antibody construct, antibody, antigen-binding portion thereof, and/or CAR as described herein can be introduced into an appropriate host cell by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment, as known to one of ordinary skill in the art.

Traditionally, monoclonal antibodies have been produced as native molecules in murine hybridoma lines. In addition to that technology, the methods and compositions described herein provide for recombinant DNA expression of monoclonal antibodies. This allows the production of humanized antibodies as well as a spectrum of antibody derivatives and fusion proteins in a host species of choice. The production of antibodies in bacteria, yeast, transgenic animals and chicken eggs are also alternatives for hybridoma-based production systems. The main advantages of transgenic animals are potential high yields from renewable sources.

In one aspect, a cell comprising an isolated antibody, antigen-binding portion thereof, or CAR as described herein is provided. In some embodiments, the isolated antibody, antigen-binding portion thereof, or CAR as described herein is expressed on the cell surface. In some embodiments, the cell comprises a nucleic acid encoding an isolated antibody, antigen-binding portion thereof, or CAR as described herein.

In some embodiments, the cell is an immune cell. As used herein, “immune cell” refers to a cell that plays a role in the immune response. Immune cells are of hematopoietic origin, and include lymphocytes, such as B-cells and T-cells; natural killer cells; myeloid cells, such as monocytes, macrophages, eosinophils, mast cells, basophils, and granulocytes. In some embodiments, the cell is a T-cell; a NK cell; a NKT-cell; lymphocytes, such as B-cells and T-cells; and myeloid cells, such as monocytes, macrophages, eosinophils, mast cells, basophils, and granulocytes.

In particular embodiments, a cell (e.g., an immune cell) is transduced with a retroviral vector, e.g., a lentiviral vector, encoding a CAR. For example, an immune effector cell is transduced with a vector encoding a CAR that comprises an anti-PD1 antibody or antigen binding portion thereof that binds a PD1 polypeptide, with an intracellular signaling domain of CD3ζ, CD28, 4-1BB, Ox40, or any combinations thereof. Thus, these transduced cells can elicit a CAR-mediated cytotoxic response.

Retroviruses are a common tool for gene delivery. In particular embodiments, a retrovirus is used to deliver a polynucleotide encoding a chimeric antigen receptor (CAR) to a cell. As used herein, the term “retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred. In particular embodiments, a lentivirus is used to deliver a polynucleotide comprising a CAR to a cell.

Retroviral vectors and more particularly lentiviral vectors may be used in practicing particular embodiments of the present invention. Accordingly, the term “retrovirus” or “retroviral vector”, as used herein is meant to include “lentivirus” and “lentiviral vectors” respectively.

In one aspect of any of the embodiments, described herein is a composition comprising an antibody, antibody reagent, antigen-binding portion thereof, or CAR as described herein or a nucleic acid encoding an antibody, antibody reagent, antigen-binding portion thereof, or CAR as described herein or a cell as described herein. In some embodiments, the composition is a pharmaceutical composition. As used herein, the term “pharmaceutical composition” refers to the active agent in combination with a pharmaceutically acceptable carrier accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance or maintain the effectiveness of the active ingredient.

The therapeutic composition as described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the invention that will be effective in the treatment of a particular disorder or condition will depend on the nature of the disorder or condition, and can be determined by standard clinical techniques.

In some embodiments, the composition comprising an antibody, antibody reagent, antigen-binding portion thereof, or CAR as described herein or a nucleic acid encoding an antibody, antibody reagent, antigen-binding portion thereof, or CAR as described herein can be a lyophilisate.

In some embodiments, the technology described herein relates to a syringe or catheter, including an organ-specific catheter (e.g., renal catheter, biliary catheter, cardiac catheter, etc.), comprising a therapeutically effective amount of a composition described herein.

As used herein, the phrase “therapeutically effective amount”, “effective amount” or “effective dose” refers to an amount that provides a therapeutic or aesthetic benefit in the treatment, prevention, or management of an autoimmune and/or autoinflammatory condition/disorder, e.g., an amount that provides a statistically significant decrease in at least one symptom, sign, or marker of the autoimmune and/or autoinflammatory condition/disorder. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

In one aspect, the technology described herein relates to a method comprising administering an antibody, antibody reagent, antigen-binding portion thereof, or CAR as described herein or a nucleic acid encoding an antibody, antibody reagent, antigen-binding portion thereof, or CAR as described herein to a subject. In some embodiments, the subject is in need of treatment for an autoimmune and/or autoinflammatory condition/disorder. In some embodiments, the method is a method of treating a subject. In some embodiments, the method is a method of treating an autoimmune and/or autoinflammatory condition/disorder in a subject.

In one aspect, described herein is a method of treating an autoimmune and/or autoinflammatory condition/disorder in a subject in need thereof, the method comprising administering a cell as described herein, e.g., a cell comprising an antibody, antibody reagent, antigen-binding portion thereof, or CAR as described herein. In some embodiments, the cell is an immune cell.

In one aspect, described herein is a method of treating an autoimmune and/or autoinflammatory condition/disorder in a subject in need thereof, the method comprising administering a nucleic acid as described herein or an immune cell comprising the nucleic acid to the subject, wherein the subject's immune cells are caused to express the polypeptide encoded by the nucleic acid. In some embodiments, the immune cell is a T-cell or is mediated by one or more T-cells. Nucleic acids can be targeted to particular cell types by, e.g., use of a cell-type specific promoter and/or a composition that selectively binds to the desired cell type. For example, conjugation of a nucleic acid to an aptamer can permit targeted delivery (McNamara, J O., et al. (2006) Nat. Biotechnol. 24:1005-1015).

In an alternative embodiment, the nucleic acid can be delivered using drug delivery systems such as a nanoparticle, a dendrimer, a polymer, liposomes, or a cationic delivery system. Positively charged cationic delivery systems facilitate binding of a nucleic acid molecule (negatively charged) and also enhance interactions at the negatively charged cell membrane to permit efficient uptake of a nucleic acid by the cell. Cationic lipids, dendrimers, or polymers can either be bound to a nucleic acid, or induced to form a vesicle or micelle (see e.g., Kim S H., et al. (2008) Journal of Controlled Release 129(2):107-116) that encases a nucleic acid. The formation of vesicles or micelles further prevents degradation of the nucleic acid when administered systemically.

Methods for making and administering cationic-inhibitory nucleic acid complexes are well within the abilities of one skilled in the art. Some non-limiting examples of drug delivery systems useful for systemic delivery of nucleic acids include DOTAP Oligofectamine, “solid nucleic acid lipid particles”, cardiolipin, polyethyleneimine, Arg-Gly-Asp (RGD) peptides, and polyamidoamines. In some embodiments, a nucleic acid forms a complex with cyclodextrin for systemic administration. Methods for administration and pharmaceutical compositions of nucleic acids and cyclodextrins can be found in U.S. Pat. No. 7,427,605, which is herein incorporated by reference in its entirety. Targeted delivery of nucleic acids is described, for example in Ikeda and Taira Pharmaceutical Res 2006 23:1631-1640; Soutschek et al., Nature 2004 432:173-8 and Lorenze et al. Bioorg. Med. Chem. Lett. 14, 4975-4977 (2004); each of which is incorporated by reference herein in its entirety. By way of example, the nucleic acid can be targeted to immune cells by encapsulating the inhibitor in a liposome comprising ligands of receptors expressed on immune cells, e.g., TCRs. In some embodiments, the liposome can comprise aptamers specific for immune cells.

In some embodiments, the methods described herein relate to CAR-T-cell therapy. CAR-T-cell and related therapies relate to adoptive cell transfer of immune cells (e.g., T-cells) expressing a CAR that binds specifically to a targeted cell type (e.g., overactive immune or inflammatory cells) to treat a subject. In some embodiments, the cells administered as part of the therapy can be autologous to the subject. In some embodiments, the cells administered as part of the therapy are not autologous to the subject. In some embodiments, the cells are engineered and/or genetically modified to express the CAR. Further discussion of CAR-T therapies can be found, e.g., in Maus et al. Blood 2014 123:2624-35; Reardon et al. Neuro-Oncology 2014 16:1441-1458; Hoyos et al. Haematologica 2012 97:1622; Byrd et al. J Clin Oncol 2014 32:3039-47; Maher et al. Cancer Res 2009 69:4559-4562; and Tamada et al. Clin Cancer Res 2012 18:6436-6445; each of which is incorporated by reference herein in its entirety.

As used herein, a “subject” means a human or animal. Usually the animal is a vertebrate such as a primate, rodent, domestic animal or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patients or subjects include any subset of the foregoing, e.g., all of the above, but excluding one or more groups or species such as humans, primates or rodents. In certain embodiments, the subject is a mammal, e.g., a primate, e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various autoimmune and/or autoinflammatory disorders. In addition, the methods described herein can be used to treat domesticated animals and/or pets. A subject can be male or female.

A subject can be one who has been previously diagnosed with or identified as suffering from or having a condition in need of treatment (e.g., an autoimmune or autoinflammatory disorder) or one or more complications related to such a condition, and optionally, but need not have already undergone treatment for a condition or the one or more complications related to the condition. Alternatively, a subject can also be one who has not been previously diagnosed as having a condition in need of treatment or one or more complications related to such a condition. For example, a subject can be one who exhibits one or more risk factors for a condition or one or more complications related to a condition or a subject who does not exhibit risk factors. A “subject in need” of treatment for a particular condition can be a subject having that condition, diagnosed as having that condition, or at risk of developing that condition.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment. Beneficial or desired clinical results include, but are not limited to, alleviation of one or more symptom(s), diminishment of extent of the deficit, stabilized (i.e., not worsening) state of a immune response, delay or slowing of disease progression, and an increased lifespan as compared to that expected in the absence of treatment.

As used herein, the term “administering,” refers to the placement of an agent, including but not limited to, an antibody, antibody reagent, antigen-binding portion thereof, or CAR as described herein or a nucleic acid encoding an antibody, antibody reagent, antigen-binding portion thereof, or CAR, or a cell comprising such an agent, as described herein into a subject by a method or route which results in at least partial localization of the agents at a desired site. The pharmaceutical composition comprising an antibody, antibody reagent, antigen-binding portion thereof, or CAR as described herein or a nucleic acid encoding an antibody, antibody reagent, antigen-binding portion thereof, or CAR, or a cell comprising such an agent as described herein disclosed herein can be administered by any appropriate route which results in an effective treatment in the subject.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into an affected joint or organ, lymph node, or site of infection.

It can generally be stated that a pharmaceutical composition comprising the cells, e.g., T-cells or immune cells, described herein may be administered at a dosage of 10² to 10¹⁰ cells/kg body weight, preferably 10⁵ to 10⁶ cells/kg body weight, including all integer values within those ranges. The number of cells will depend upon the ultimate use for which the composition is intended as will the type of cells included therein. For uses provided herein, the cells are generally in a volume of a liter or less, can be 500 mLs or less, even 250 mLs or 100 mLs or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells.

In some aspects of the present invention, particularly since all the infused cells will be redirected to a particular target antigen, lower numbers of cells, in the range of 10⁶/kilogram (10⁶-10¹¹ per patient) may be administered. CAR expressing cell compositions may be administered multiple times at dosages within these ranges. In some embodiments, the dosage can be from about 1×10⁵ cells to about 1×10⁸ cells per kg of body weight. In some embodiments, the dosage can be from about 1×10⁶ cells to about 1×10⁷ cells per kg of body weight. In some embodiments, the dosage can be about 1×10⁶ cells per kg of body weight. In some embodiments, one dose of cells can be administered. In some embodiments, the dose of cells can be repeated, e.g., once, twice, or more. In some embodiments, the dose of cells can be administered on, e.g., a daily, weekly, or monthly basis.

The dosage ranges for the agent depend upon the potency, and encompass amounts large enough to produce the desired effect e.g., slowing of disease progression or a reduction in disease activity. The dosage should not be so large as to cause unacceptable adverse side effects. Generally, the dosage will vary with the age, condition, and sex of the patient and can be determined by one of skill in the art. The dosage can also be adjusted by the individual physician in the event of any complication. In some embodiments, the dosage ranges from 0.001 mg/kg body weight to 0.5 mg/kg body weight. In some embodiments, the dose range is from 5 μg/kg body weight to 100 μg/kg body weight. Alternatively, the dose range can be titrated to maintain serum levels between 1 μg/mL and 1000 μg/mL. For systemic administration, subjects can be administered a therapeutic amount, such as, e.g., 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

Administration of the doses recited above can be repeated. In some embodiments, the doses are given once a day, or multiple times a day, for example but not limited to three times a day. In some embodiments, the doses recited above are administered daily for several weeks or months. The duration of treatment depends upon the subject's clinical progress and responsiveness to therapy.

In some embodiments, the dose can be from about 2 mg/kg to about 15 mg/kg. In some embodiments, the dose can be about 2 mg/kg. In some embodiments, the dose can be about 4 mg/kg. In some embodiments, the dose can be about 5 mg/kg. In some embodiments, the dose can be about 6 mg/kg. In some embodiments, the dose can be about 8 mg/kg. In some embodiments, the dose can be about 10 mg/kg. In some embodiments, the dose can be about 15 mg/kg. In some embodiments, the dose can be from about 100 mg/m² to about 700 mg/m². In some embodiments, the dose can be about 250 mg/m². In some embodiments, the dose can be about 375 mg/m². In some embodiments, the dose can be about 400 mg/m². In some embodiments, the dose can be about 500 mg/m².

In some embodiments, the dose can be administered intravenously. In some embodiments, the intravenous administration can be an infusion occurring over a period of from about 10 minute to about 3 hours. In some embodiments, the intravenous administration can be an infusion occurring over a period of from about 30 minutes to about 90 minutes.

In some embodiments, the doses are given twice a week, once a week, bikweekly, or monthly. In some embodiments, the dose can be administered weekly for from about 12 weeks to about 18 weeks. In some embodiments the dose can be administered about every 2 weeks. In some embodiments the dose can be administered about every 3 weeks. In some embodiments, the dose can be from about 2 mg/kg to about 15 mg/kg administered about every 2 weeks. In some embodiments, the dose can be from about 2 mg/kg to about 15 mg/kg administered about every 3 weeks. In some embodiments, the dose can be from about 2 mg/kg to about 15 mg/kg administered intravenously about every 2 weeks. In some embodiments, the dose can be from about 2 mg/kg to about 15 mg/kg administered intravenously about every 3 weeks. In some embodiments, the dose can be from about 200 mg/m² to about 400 mg/m² administered intravenously about every week. In some embodiments, the dose can be from about 200 mg/m² to about 400 mg/m² administered intravenously about every 2 weeks. In some embodiments, the dose can be from about 200 mg/m² to about 400 mg/m² administered intravenously about every 3 weeks. In some embodiments, a total of from about 2 to about 10 doses are administered. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, the administration occurs for a total of from about 4 weeks to about 12 weeks. In some embodiments, the administration occurs for a total of about 6 weeks. In some embodiments, the administration occurs for a total of about 8 weeks. In some embodiments, the administration occurs for a total of about 12 weeks. In some embodiments, the initial dose can be from about 1.5 to about 2.5 fold greater than subsequent doses.

In some embodiments, the dose can be from about 1 mg to about 2000 mg. In some embodiments, the dose can be about 3 mg. In some embodiments, the dose can be about 10 mg. In some embodiments, the dose can be about 30 mg. In some embodiments, the dose can be about 1000 mg. In some embodiments, the dose can be about 2000 mg. In some embodiments, the dose can be about 3 mg given by intravenous infusion daily. In some embodiments, the dose can be about 10 mg given by intravenous infusion daily. In some embodiments, the dose can be about 30 mg given by intravenous infusion three times per week.

A therapeutically effective amount is an amount of an agent that is sufficient to produce a statistically significant, measurable change in disease activity, disease progression, etc. (efficacy measurements are described below herein). Such effective amounts can be gauged in clinical trials as well as animal studies.

An agent can be administered intravenously by injection or by gradual infusion over time. Given an appropriate formulation for a given route, for example, agents useful in the methods and compositions described herein can be administered intravenously, intranasally, by inhalation, intraperitoneally, intramuscularly, subcutaneously, intracavity, and can be delivered by peristaltic means, if desired, or by other means known by those skilled in the art. It is preferred that the compounds used herein are administered orally, intravenously or intramuscularly to a patient having an autoimmune or autoinflammatory response, condition, or disorder. Local administration directly to affected sites is also specifically contemplated.

Therapeutic compositions containing at least one agent can be conventionally administered in a unit dose, for example. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

In some embodiments, the methods further comprise administering the pharmaceutical composition described herein along with one or more additional immunosuppressive agents, biologics, drugs, or treatments as part of a combinatorial therapy. In some such embodiments, the immunosuppressive agent biologic, drug, or treatment is selected from the group consisting of: disease-modifying anti-rheumatic drugs (DMARDs), surgery, antibody reagents, and/or small molecules.

In some embodiments of the methods described herein, the methods further comprise administering one or more immunosuppressive agents to the subject being administered the pharmaceutical composition described herein. Non-limiting examples of immunosuppressive agents can include calcineurin inhibitors such as cyclosporine, and tacrolimus; interleukin inhibitors such as canakinumab, rilonacept, brodalumab, reslizumab, anakinra, ustekinumab, benralizumab, ixekizumab, dupilumab, mepolizumab, tocilizumab, guselkumab, secukinumab, tildrakizumab, sarilumab, basiliximab, daclizumab, risankizumab, and siltuximab; selective immunosuppressants such as alefacept, sirolimus, mycophenolic acid, mycophenolate mofetil, belatacept, belimumab, natalizumab, fingolimod, dimethyl fumarate, everolimus, abatacept, teriflunomide, vedolizumab, leflunomide, anti-thymocyte globulin, baricitinib, diroximel fumarate, eculizumab, emapalumab, lymphocyte immune globulin, monomethyl fumarate, muromonab-cd3, ozanimod, ravulizumab, and siponimod; TNF alfa inhibitors such as adalimumab, certolizumab, etanercept, golimumab, and infliximab; other immunosuppressants such as azathioprine, lenalidomide, methotrexate, omalizumab, pomalidomide, and thalidomide; and pharmaceutically acceptable salts, acids or derivatives of any one of the above.

The efficacy of a given treatment for, e.g., autoimmune or autoinflammatory disorders, can be determined by the skilled clinician. However, a treatment is considered “effective treatment,” as the term is used herein, if any one or all of the signs or symptoms of e.g., the autoimmune or autoinflammatory disorder is treated and/or managed in a beneficial manner or other clinically accepted symptoms are improved, e.g., by at least 10% following treatment with an agent as described herein. Efficacy can also be measured by a failure of an individual to worsen as assessed by hospitalization or need for medical interventions (i.e., progression of the disease is halted). Methods of measuring these indicators are known to those of skill in the art and/or described herein. An effective amount for the treatment of a disease means that amount which, when administered to a mammal in need thereof, is sufficient to result in effective treatment as that term is defined herein, for that disease. Efficacy of an agent can be determined by assessing physical indicators of disease progression, disease activity, etc.

In one aspect, described herein is a method of detecting PD1, the method comprising contacting a biological sample with an antibody, antibody reagent, or antigen-binding portion thereof as described herein, wherein reaction of the antibody or antigen-binding portion thereof with PD1 indicates the presence of PD1. In some embodiments, a detectable signal is generated by the antibody or antigen-binding portion thereof when a PD1 molecule is present. In some embodiments, the antibody or antigen-binding portion thereof is detectably labeled or capable of generating a detectable signal. In some embodiments, the level of the PD1 is determined using a method selected from the group consisting of Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluoresence assay; mass spectroscopy; FACS; and immunoelectrophoresis assay. In some embodiments, the antibody or antigen-binding portion thereof is detectably labeled or generates a detectable signal. In some embodiments, the expression level of PD1 is normalized relative to the expression level of one or more reference genes or reference proteins. In some embodiments, the reference level of PD1 is the expression level of PD1 in a prior sample obtained from the subject.

In some embodiments, the level of PD1 can be the level of PD1 polypeptide. Detection of PD1 polypeptides can be according to any method known in the art. Immunological methods to detect PD1 polypeptides in accordance with the present technology include, but are not limited to antibody techniques such as immunohistochemistry, immunocytochemistry, flow cytometry, fluorescence-activated cell sorting (FACS), immunoblotting, radioimmunoassays, western blotting, immunoprecipitation, enzyme-linked immunosorbant assays (ELISA), and derivative techniques that make use of antibody reagents as described herein.

Immunochemical methods require the use of an antibody reagent specific for the target molecule (e.g., the antigen or in the embodiments described herein, a PD1 polypeptide. In some embodiments, the assays, methods, and/or systems described herein can comprise: an anti-PD1 antibody reagent. In some embodiments, the antibody reagent can be detectably labeled. In some embodiments, the antibody reagent can be attached to a solid support (e.g., bound to a solid support). In some embodiments, the solid support can comprise a particle (including, but not limited to an agarose or latex bead or particle or a magnetic particle), a bead, a nanoparticle, a polymer, a substrate, a slide, a coverslip, a plate, a dish, a well, a membrane, and/or a grating. The solid support can include many different materials including, but not limited to, polymers, plastics, resins, polysaccharides, silicon or silica based materials, carbon, metals, inorganic glasses, and membranes.

In one embodiment, an assay, method, and/or system as described herein can comprise an ELISA. In an exemplary embodiment, a first antibody reagent can be immobilized on a solid support (usually a polystyrene micro titer plate). The solid support can be contacted with a sample obtained from a subject, and the antibody reagent will bind (“capture”) antigens for which it is specific (e.g., PD1). The solid support can then be contacted with a second labeled antibody reagent (e.g., a detection antibody reagent). The detection antibody reagent can, e.g., comprise a detectable signal, be covalently linked to an enzyme, or can itself be detected by a secondary antibody which is linked to an enzyme through bio-conjugation. The presence of a signal indicates that both the first antibody reagent immobilized on the support and the second “detection” antibody reagent have bound to an antigen, i.e., the presence of a signal indicated the presence of a PD1 molecule. Between each step the plate is typically washed with a mild detergent solution to remove any proteins or antibodies that are not specifically bound. After the final wash step, the plate is developed by adding an enzymatic substrate to produce a visible signal, which indicates the quantity of PD1 polypeptides in the sample. Older ELISAs utilize chromogenic substrates, though newer assays employ fluorogenic substrates with much higher sensitivity. There are other different forms of ELISA, which are well known to those skilled in the art.

In one embodiment, the assays, systems, and methods described herein can comprise a lateral flow immunoassay test (LFIA), also known as the immunochromatographic assay, or strip test to measure or determine the level of PD1 polypeptide in a sample. LFIAs are a simple device intended to detect the presence (or absence) of PD1 in a sample. There are currently many LFIA tests used for medical diagnostics either for home testing, point of care testing, or laboratory use. LFIA tests are a form of immunoassay in which the test sample flows along a solid substrate via capillary action. After the sample is applied to the test strip it encounters a colored antibody reagent which mixes with the sample, and if bound to a portion of the sample, transits the substrate encountering lines or zones which have been pretreated with a second antibody reagent. Depending upon the level of PD1 present in the sample the colored antibody reagent can become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water samples etc. Strip tests are also known as dip stick test, the name bearing from the literal action of “dipping” the test strip into a fluid sample to be tested. LFIA strip test are easy to use, require minimum training and can easily be included as components of point-of-care test (POCT) diagnostics to be used on site in the field. LFIA tests can be operated as either competitive or sandwich assays. Sandwich LFIAs are similar to sandwich ELISA. The sample first encounters colored particles which are labeled with antibody reagents specific for a target (e.g., a PD1-specific antibody reagent). The test line will also contain antibody reagents (e.g., a PD1-specific antibody reagent). The test line will show as a colored band in positive samples. In some embodiments, the lateral flow immunoassay can be a double antibody sandwich assay, a competitive assay, a quantitative assay or variations thereof. There are a number of variations on lateral flow technology. It is also possible to apply multiple capture zones to create a multiplex test.

A typical test strip consists of the following components: (1) sample application area comprising an absorbent pad (i. e. the matrix or material) onto which the test sample is applied; (2) conjugate or reagent pad—this contains antibody reagent(s) specific to the target which can be conjugated to colored particles (usually colloidal gold particles, or latex microspheres); (3) test results area comprising a reaction membrane—typically a hydrophobic nitrocellulose or cellulose acetate membrane onto which antibody reagents are immobilized in a line across the membrane as a capture zone or test line (a control zone may also be present, containing antibodies specific for the antibody reagents conjugated to the particles or microspheres); and (4) optional wick or waste reservoir—a further absorbent pad designed to draw the sample across the reaction membrane by capillary action and collect it. The components of the strip are usually fixed to an inert backing material and may be presented in a simple dipstick format or within a plastic casing with a sample port and reaction window showing the capture and control zones. While not strictly necessary, most tests will incorporate a second line which contains an antibody that picks up free latex/gold in order to confirm the test has operated correctly.

The use of “dip sticks” or LFIA test strips and other solid supports has been described in the art in the context of an immunoassay for a number of antigen biomarkers. U.S. Pat. Nos. 4,943,522; 6,485,982; 6,187,598; 5,770,460; 5,622,871; 6,565,808, U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Three U.S. patents (U.S. Pat. No. 4,444,880, issued to H. Tom; U.S. Pat. No. 4,305,924, issued to R. N. Piasio; and U.S. Pat. No. 4,135,884, issued to J. T. Shen) describe the use of “dip stick” technology to detect soluble antigens via immunochemical assays. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teaching of these “dip stick” technologies as necessary for the detection of PD1 polypeptides. In some embodiments, the dip stick (or LFIA) can be suitable for use with urine samples. In some embodiments, a dip stick can be suitable for use with blood samples.

Immunochemistry is a family of techniques based on the use of a specific antibody, wherein antibodies are used to specifically target molecules inside or on the surface of cells. In some embodiments, immunohistochemistry (“IHC”) and immunocytochemistry (“ICC”) techniques can be used to detect or measure the levels of PD1 polypeptide. IHC is the application of immunochemistry to tissue sections, whereas ICC is the application of immunochemistry to cells or tissue imprints after they have undergone specific cytological preparations such as, for example, liquid-based preparations. In some instances, signal amplification may be integrated into the particular protocol, wherein a secondary antibody, that includes a label, follows the application of an antibody reagent specific for platelets or leukocytes. Typically, for immunohistochemistry, tissue obtained from a subject and fixed by a suitable fixing agent such as alcohol, acetone, and paraformaldehyde, is sectioned and reacted with an antibody. Conventional methods for immunohistochemistry are described in Buchwalow and Bocker (Eds) “Immunohistochemistry: Basics and Methods” Springer (2010): Lin and Prichard “Handbook of Practical Immunohistochemistry” Springer (2011); which are incorporated by reference herein in their entireties. In some embodiments, immunocytochemistry may be utilized where, in general, tissue or cells obtained from a subject are fixed by a suitable fixing agent such as alcohol, acetone, and paraformaldehyde, to which is reacted an antibody. Methods of immunocytological staining of human samples is known to those of skill in the art and described, for example, in Burry “Immunocytochemistry: A Practical Guide for Biomedical Research” Springer (2009); which is incorporated by reference herein in its entirety.

In some embodiments, one or more of the antibody reagents described herein can comprise a detectable label and/or comprise the ability to generate a detectable signal (e.g., by catalyzing a reaction converting a compound to a detectable product). Detectable labels can comprise, for example, a light-absorbing dye, a fluorescent dye, or a radioactive label. Detectable labels, methods of detecting them, and methods of incorporating them into an antibody reagent are well known in the art.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the antibody reagent. Alternatively, a detectable label can be linked such as by directly labeling a molecule that achieves binding to the antibody reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection antibody is labeled with a fluorescent compound. When the fluorescently labeled antibody is exposed to light of the proper wavelength, its presence can then be detected due to fluorescence. In some embodiments, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g., Cy3, Cy5 and Cy7 dyes; coumarins, e.g umbelliferone; benzimide dyes, e.g., Hoechst 33258; phenanthridine dyes, e.g., Texas Red; ethidium dyes; acridine dyes; carbazole dyes; phenoxazine dyes; porphyrin dyes; polymethine dyes, e.g., cyanine dyes such as Cy3, Cy5, etc; BODIPY dyes and quinoline dyes.

In some embodiments, a detectable label can be a radiolabel including, but not limited to ³H, ¹²⁵I, ³⁵S, ¹⁴C, ³²P, and ³³P.

In some embodiments, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase.

In some embodiments, a detectable label is a chemiluminescent label, including, but not limited to lucigenin, luminol, luciferin, isoluminol, theromatic acridinium ester, imidazole, acridinium salt and oxalate ester. In some embodiments, a detectable label can be a spectral colorimetric label including, but not limited to colloidal gold or colored glass or plastic (e.g., polystyrene, polypropylene, and latex) beads.

In some embodiments, antibodies can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e. g. from DAKO; Carpinteria, CA.

An antibody reagent can also be detectably labeled using fluorescence emitting metals such as ¹⁵²Eu, or others of the lanthanide series. These metals can be attached to the antibody reagent using such metal chelating groups as diethylenetriaminepentaacetic acid (DTPA) or ethylenediaminetetraacetic acid (EDTA).

The term “sample” or “test sample” as used herein denotes a sample taken or isolated from an organism, e.g., a urine sample from a subject. Exemplary biological samples include, but are not limited to, a biofluid sample; serum; plasma; urine; saliva; and/or joint fluid sample, etc. The term also includes a mixture of the above-mentioned samples. The term “test sample” also includes untreated or pretreated (or pre-processed) biological samples. In some embodiments, a test sample can comprise cells from a subject. As used herein, the term “biofluid” refers to any fluid obtained from a biological source and includes, but is not limited to, blood, urine, and bodily secretions.

The test sample can be obtained by removing a sample from a subject, but can also be accomplished by using a previously isolated sample (e.g., isolated at a prior timepoint and isolated by the same or another person). In addition, the test sample can be freshly collected or a previously collected sample.

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, prepared by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the level of PD1 as described herein.

In some embodiments, the methods, assays, and systems described herein can further comprise a step of obtaining a test sample from a subject. In some embodiments, the subject can be a human subject.

In some embodiments, the assay or method can further comprise the step of administering an anti-PD1 therapy. In some embodiments, the anti-PD1 therapy comprises an isolated antibody, antibody reagent, antigen-binding portion thereof, or CAR or CAR T-cell; nucleic acid; cell; or composition as described herein.

In one aspect of any of any of the embodiments, described herein is an antibody, antibody reagent, or antigen-binding portion thereof as described herein conjugated to or coupled to a detectable label.

In one aspect of any of any of the embodiments, described herein is a solid support comprising an antibody, antibody reagent, antigen-binding fragment thereof as described herein. In some embodiments of any of the aspects, the antibody, antibody reagent or antigen-binding fragment thereof is detectably labeled. In some embodiments of any of the aspects, the solid support comprises a particle, a bead, a polymer, or a substrate.

In one aspect of any of the embodiments, described herein is a molecular complex comprising at least one antibody, antibody reagent, antigen-binding fragment thereof, or CAR of as described herein bound to an PD1 polypeptide.

In one aspect, described herein is a kit comprising a composition as described herein, e.g., a composition comprising an antibody, antibody reagent, antigen-binding portion thereof, or CAR as described herein. A kit is any manufacture (e.g., a package or container) comprising at least one reagent, e.g., an antibody, the manufacture being promoted, distributed, or sold as a unit for performing the methods described herein. In some embodiments of any of the aspects, the antibody, antibody reagent, antigen-binding fragment thereof as described herein is immobilized on a solid support. In some embodiments of any of the aspects, the solid support comprises a particle, a bead, a polymer, or a substrate. In some embodiments of any of the aspects, the antibody, antibody reagent or antigen-binding fragment thereof is detectably labeled.

The kits described herein can optionally comprise additional components useful for performing the methods described herein. By way of example, the kit can comprise fluids (e.g., buffers) suitable for composition comprising an antibody, antigen-binding portion thereof, or CAR as described herein, an instructional material which describes performance of a method as described herein, and the like. A kit can further comprise devices and/or reagents for delivery of the composition as described herein. Additionally, the kit may comprise an instruction leaflet and/or may provide information as to the relevance of the obtained results.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell is typically still referred to as “engineered” even though the actual manipulation was performed on a prior entity.

In some embodiments, a nucleic acid encoding a polypeptide as described herein (e.g. an antibody or antibody reagent) is comprised by a vector. In some of the aspects described herein, a nucleic acid sequence encoding a given polypeptide as described herein, or any module thereof, is operably linked to a vector. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.” The terms “purified” or “substantially purified” refer to an isolated nucleic acid or polypeptide that is at least 95% by weight the subject nucleic acid or polypeptide, including, for example, at least 96%, at least 97%, at least 98%, at least 99% or more. In some embodiments, the antibody, antigen-binding portion thereof, or CAR described herein is isolated. In some embodiments, the antibody, antibody reagent, antigen-binding portion thereof, or CAR described herein is purified.

As used herein, “engineered” refers to the aspect of having been manipulated by the hand of man. For example, an antibody, antibody reagent, antigen-binding portion thereof, or CAR is considered to be “engineered” when the sequence of the antibody, antibody reagent, antigen-binding portion thereof, or CAR is manipulated by the hand of man to differ from the sequence of an antibody as it exists in nature. As is common practice and is understood by those in the art, progeny and copies of an engineered polynucleotide and/or polypeptide are typically still referred to as “engineered” even though the actual manipulation was performed on a prior entity.

As used herein, an “epitope” can be formed on a polypeptide both from contiguous amino acids, or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An “epitope” includes the unit of structure conventionally bound by an immunoglobulin VH/VL pair. Epitopes define the minimum binding site for an antibody, and thus represent the target of specificity of an antibody. In the case of a single domain antibody, an epitope represents the unit of structure bound by a variable domain in isolation. The terms “antigenic determinant” and “epitope” can also be used interchangeably herein. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics.

“Avidity” is the measure of the strength of binding between an antigen-binding molecule (such as an antibody or antigen-binding portion thereof described herein) and the pertinent antigen. Avidity is related to both the affinity between an antigenic determinant and its antigen binding site on the antigen-binding molecule, and the number of pertinent binding sites present on the antigen-binding molecule. Typically, antigen-binding proteins (such as an antibody or portion of an antibody as described herein) will bind to their cognate or specific antigen with a dissociation constant (KD of 10⁻⁵ to 10⁻¹² moles/liter or less, such as 10⁻⁷ to 10⁻¹² moles/liter or less, or 10⁻⁸ to 10⁻¹² moles/liter (i.e., with an association constant (KA) of 10⁵ to 10¹² liter/moles or more, such as 10⁷ to 10¹² liter/moles or 10⁸ to 10¹² liter/moles). Any KD value greater than 10⁻⁴ mol/liter (or any KA value lower than 10⁴ M⁻¹) is generally considered to indicate non-specific binding. The KD for biological interactions which are considered meaningful (e.g., specific) are typically in the range of 10⁻¹⁰ M (0.1 nM) to 10⁻⁵ M (10000 nM). The stronger an interaction, the lower is its KD. For example, a binding site on an antibody or portion thereof described herein will bind to the desired antigen with an affinity less than 500 nM, such as less than 200 nM, or less than 10 nM, such as less than 500 pM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art; as well as other techniques as mentioned herein.

Accordingly, as used herein, “selectively binds” or “specifically binds” refers to the ability of a peptide (e.g., an antibody, CAR or portion thereof) described herein to bind to a target, such as an antigen present on the cell-surface, with a KD 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay. A polypeptide specifically bound to a target is not displaced by a non-similar competitor. In certain embodiments, an antibody, antigen-binding portion thereof, or CAR is said to specifically bind an antigen when it preferentially recognizes its target antigen in a complex mixture of proteins and/or macromolecules.

In some embodiments, an antibody, antigen-binding portion thereof, and/or CAR as described herein binds to PD1 with a dissociation constant (K_{D}) of 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. In some embodiments, an antibody, antigen-binding portion thereof, and/or CAR as described herein binds to PD1 with a dissociation constant (K_{D}) of from about 10⁻⁵ M to 10⁻⁶ M. In some embodiments, an antibody, antigen-binding portion thereof, and/or CAR as described herein binds to PD1 with a dissociation constant (K_{D}) of from about 10⁻⁶ M to 10⁻⁷ M. In some embodiments, an antibody, antigen-binding portion thereof, and/or CAR as described herein binds to PD1 with a dissociation constant (K_{D}) of from about 10⁻⁷ M to 10⁻¹ M. In some embodiments, an antibody, antigen-binding portion thereof, and/or CAR as described herein binds to PD1 with a dissociation constant (K_{D}) of from about 10⁻⁸, M to 10⁻⁹ M. In some embodiments, an antibody, antigen-binding portion thereof, and/or CAR as described herein binds to PD1 with a dissociation constant (K_{D}) of from about 10⁻⁹ M to 10⁻¹⁰ M. In some embodiments, an antibody, antigen-binding portion thereof, and/or CAR as described herein binds to PD1 with a dissociation constant (K_{D}) of from about 10⁻¹⁰ M to 10⁻¹ M. In some embodiments, an antibody, antigen-binding portion thereof, and/or CAR as described herein binds to PD1 with a dissociation constant (K_{D}) of from about 10⁻¹¹ M to 10⁻¹² M. In some embodiments, an antibody, antigen-binding portion thereof, and/or CAR as described herein binds to PD1 with a dissociation constant (K_{D}) of less than 10⁻¹² M.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:

1. An antibody, antibody reagent, antigen-binding fragment thereof, or chimeric antigen receptor (CAR), that specifically binds an PD1 polypeptide, said antibody reagent, antigen-binding portion thereof, or CAR comprising at least one heavy or light chain complementarity determining region (CDR) selected from the group consisting of:
(a) a heavy chain CDR1 having the amino acid sequence of SEQ ID NO: 3;
(b) a heavy chain CDR2 having the amino acid sequence of SEQ ID NO: 4;
(c) a heavy chain CDR3 having the amino acid sequence of SEQ ID NO: 5;
(d) a light chain CDR1 having the amino acid sequence of SEQ ID NO: 6;
(e) a light chain CDR2 having the amino acid sequence of SEQ ID NO: 7; and
(f) a light chain CDR3 having the amino acid sequence of SEQ ID NO: 8; or
   selected from the group consisting of:
(a) a heavy chain CDR1 having the amino acid sequence of SEQ ID NO: 19;
(b) a heavy chain CDR2 having the amino acid sequence of SEQ ID NO: 20;
(c) a heavy chain CDR3 having the amino acid sequence of SEQ ID NO: 21;
(d) a light chain CDR1 having the amino acid sequence of SEQ ID NO: 22;
(e) a light chain CDR2 having the amino acid sequence of SEQ ID NO: 23; and
(f) a light chain CDR3 having the amino acid sequence of SEQ ID NO: 24; or
   a conservative substitution variant of one or more of (a)-(f).

2. The antibody, antibody reagent, antigen-binding portion thereof, or CAR of paragraph 1, which comprises heavy chain CDRs having the amino acid sequences of SEQ ID NOs: 3-5, or 19-21, or a conservative substitution variant of such amino acid sequence.

3. The antibody, antibody reagent, antigen-binding portion thereof, or CAR of any one of paragraphs 1-2, which comprises light chain CDRs having the amino acid sequences of SEQ ID NOs: 6-8, or 22-24, or a conservative substitution variant of such amino acid sequence.

4. The antibody, antibody reagent, antigen-binding portion thereof, or CAR of any one of paragraphs 1-3, which comprises:
heavy chain CDRs having the amino acid sequences of SEQ ID NOs: 3-5 and light chain CDRs having the amino acid sequences of SEQ ID NOs: 6-8 or a conservative substitution variant of such amino acid sequence; or
heavy chain CDRs having the amino acid sequences of SEQ ID NOs: 19-21 and light chain CDRs having the amino acid sequences of SEQ ID NOs: 22-24 or a conservative substitution variant of such amino acid sequence.

5. A first antibody, antibody reagent, antigen-binding portion thereof, or CAR that specifically binds an PD1 polypeptide, and can compete for binding of PD1 with a second antibody comprising:
heavy chain CDRs having the amino acid sequences of SEQ ID NOs: 3-5 and light chain CDRs having the amino acid sequences of SEQ ID NOs: 6-8 or a conservative substitution variant of such amino acid sequence; or
heavy chain CDRs having the amino acid sequences of SEQ ID NOs: 19-21 and light chain CDRs having the amino acid sequences of SEQ ID NOs: 22-24 or a conservative substitution variant of such amino acid sequence.

6. The first antibody, antibody reagent, antigen-binding fragment thereof, or chimeric antigen receptor (CAR) of paragraph 5, comprising at least one heavy or light chain complementarity determining region (CDR) selected from the group consisting of:
(a) a heavy chain CDR1 having the amino acid sequence of SEQ ID NO: 3;
(b) a heavy chain CDR2 having the amino acid sequence of SEQ ID NO: 4;
(c) a heavy chain CDR3 having the amino acid sequence of SEQ ID NO: 5;
(d) a light chain CDR1 having the amino acid sequence of SEQ ID NO: 6;
(e) a light chain CDR2 having the amino acid sequence of SEQ ID NO: 7; and
(f) a light chain CDR3 having the amino acid sequence of SEQ ID NO: 8; or
   selected from the group consisting of:
(a) a heavy chain CDR1 having the amino acid sequence of SEQ ID NO: 19;
(b) a heavy chain CDR2 having the amino acid sequence of SEQ ID NO: 20;
(c) a heavy chain CDR3 having the amino acid sequence of SEQ ID NO: 21;
(d) a light chain CDR1 having the amino acid sequence of SEQ ID NO: 22;
(e) a light chain CDR2 having the amino acid sequence of SEQ ID NO: 23; and
(f) a light chain CDR3 having the amino acid sequence of SEQ ID NO: 24; or
   a conservative substitution variant of one or more of (a)-(f).

7. The antibody, antibody reagent, antigen-binding portion thereof, or CAR of any one of paragraphs 5-6, which comprises heavy chain CDRs having the amino acid sequences of SEQ ID NOs: 3-5, or 19-21, or a conservative substitution variant of such amino acid sequence.

8. The antibody, antibody reagent, antigen-binding portion thereof, or CAR of any one of paragraphs 5-7, which comprises light chain CDRs having the amino acid sequences of SEQ ID NOs: 6-8, or 22-24, or a conservative substitution variant of such amino acid sequence.

9. The antibody, antibody reagent, antigen-binding portion thereof, or CAR of any one of paragraphs 5-8, which comprises:
heavy chain CDRs having the amino acid sequences of SEQ ID NOs: 3-5 and light chain CDRs having the amino acid sequences of SEQ ID NOs: 6-8 or a conservative substitution variant of such amino acid sequence, or
heavy chain CDRs having the amino acid sequences of SEQ ID NOs: 19-21 and light chain CDRs having the amino acid sequences of SEQ ID NOs: 22-24 or a conservative substitution variant of such amino acid sequence.

10. The antibody, antibody reagent, antigen-binding portion thereof, or CAR of any one of paragraphs 1-9, comprising the heavy chain variable region sequence of SEQ ID NO: 1 or 17.

11. The antibody, antibody reagent, antigen-binding portion thereof, or CAR of any one of paragraphs 1-10, comprising the light chain variable region sequence of SEQ ID NO: 2 or 18.

12. The antibody, antibody reagent, antigen-binding portion thereof, or CAR of any one of paragraphs 1-11, comprising:
the heavy chain variable region sequence of SEQ ID NO: 1 and the light chain variable region sequence of SEQ ID NO: 2; or
the heavy chain variable region sequence of SEQ ID NO: 17 and the light chain variable region sequence of SEQ ID NO: 18.

13. The antibody, antibody reagent, antigen-binding portion thereof, or CAR of any one of paragraphs 1-12, further comprising a conservative substitution in a sequence not comprised by a CDR.

14. The antibody, antibody reagent, antigen-binding portion thereof, or CAR of any one of paragraphs 1-13, wherein the antibody reagent or antigen-binding fragment thereof is fully human or fully humanized.

15. The antibody, antibody reagent, antigen-binding portion thereof, or CAR of any one of paragraphs 1-14, wherein the antibody reagent or antigen-binding fragment thereof is fully humanized except for the CDR sequences.

16. The antibody, antibody reagent, antigen-binding portion thereof, or CAR of any one of paragraphs 1-15, wherein the antibody reagent or antigen-binding fragment is selected from the group consisting of:
an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, and a bispecific antibody.

17. A composition, kit, or combination comprising:
(i) the antibody, antibody reagent, antigen-binding portion thereof, or CAR of any one of paragraphs 1-16 or an antibody, antibody reagent, antigen-binding portion thereof, or CAR comprising at least one heavy or light chain complementarity determining region (CDR) selected from the group consisting of:
   (a) a heavy chain CDR1 having the amino acid sequence of SEQ ID NO: 11;
   (b) a heavy chain CDR2 having the amino acid sequence of SEQ ID NO: 12;
   (c) a heavy chain CDR3 having the amino acid sequence of SEQ ID NO: 13;
   (d) a light chain CDR1 having the amino acid sequence of SEQ ID NO: 14;
   (e) a light chain CDR2 having the amino acid sequence of SEQ ID NO: 15; and
   (f) a light chain CDR3 having the amino acid sequence of SEQ ID NO: 16, or
selected from the group consisting of:
   (a) a heavy chain CDR1 having the amino acid sequence of SEQ ID NO: 27;
   (b) a heavy chain CDR2 having the amino acid sequence of SEQ ID NO: 28;
   (c) a heavy chain CDR3 having the amino acid sequence of SEQ ID NO: 29;
   (d) a light chain CDR1 having the amino acid sequence of SEQ ID NO: 30;
   (e) a light chain CDR2 having the amino acid sequence of SEQ ID NO: 31; and
   (f) a light chain CDR3 having the amino acid sequence of SEQ ID NO: 32, or
selected from the group consisting of:
   (a) a heavy chain CDR1 having the amino acid sequence of SEQ ID NO: 35;
   (b) a heavy chain CDR2 having the amino acid sequence of SEQ ID NO: 36;
   (c) a heavy chain CDR3 having the amino acid sequence of SEQ ID NO: 37;
   (d) a light chain CDR1 having the amino acid sequence of SEQ ID NO: 38;
   (e) a light chain CDR2 having the amino acid sequence of SEQ ID NO: 39; and
   (f) a light chain CDR3 having the amino acid sequence of SEQ ID NO: 40, or
selected from the group consisting of:
   (a) a heavy chain CDR1 having the amino acid sequence of SEQ ID NO: 43;
   (b) a heavy chain CDR2 having the amino acid sequence of SEQ ID NO: 44;
   (c) a heavy chain CDR3 having the amino acid sequence of SEQ ID NO: 45;
   (d) a light chain CDR1 having the amino acid sequence of SEQ ID NO: 46;
   (e) a light chain CDR2 having the amino acid sequence of SEQ ID NO: 47; and
   (f) a light chain CDR3 having the amino acid sequence of SEQ ID NO: 48, or
selected from the group consisting of:
   (a) a heavy chain CDR1 having the amino acid sequence of SEQ ID NO: 51;
   (b) a heavy chain CDR2 having the amino acid sequence of SEQ ID NO: 52;
   (c) a heavy chain CDR3 having the amino acid sequence of SEQ ID NO: 53;
   (d) a light chain CDR1 having the amino acid sequence of SEQ ID NO: 54;
   (e) a light chain CDR2 having the amino acid sequence of SEQ ID NO: 55; and
   (f) a light chain CDR3 having the amino acid sequence of SEQ ID NO: 56, or
selected from the group consisting of:
   (a) a heavy chain CDR1 having the amino acid sequence of SEQ ID NO: 59;
   (b) a heavy chain CDR2 having the amino acid sequence of SEQ ID NO: 60;
   (c) a heavy chain CDR3 having the amino acid sequence of SEQ ID NO: 61;
   (d) a light chain CDR1 having the amino acid sequence of SEQ ID NO: 62;
   (e) a light chain CDR2 having the amino acid sequence of SEQ ID NO: 63; and
   (f) a light chain CDR3 having the amino acid sequence of SEQ ID NO: 64, or
selected from the group consisting of:
   (a) a heavy chain CDR1 having the amino acid sequence of SEQ ID NO: 67;
   (b) a heavy chain CDR2 having the amino acid sequence of SEQ ID NO: 68;
   (c) a heavy chain CDR3 having the amino acid sequence of SEQ ID NO: 69;
   (d) a light chain CDR1 having the amino acid sequence of SEQ ID NO: 70;
   (e) a light chain CDR2 having the amino acid sequence of SEQ ID NO: 71; and
   (f) a light chain CDR3 having the amino acid sequence of SEQ ID NO: 72, or
selected from the group consisting of:
   (a) a heavy chain CDR1 having the amino acid sequence of SEQ ID NO: 75;
   (b) a heavy chain CDR2 having the amino acid sequence of SEQ ID NO: 76;
   (c) a heavy chain CDR3 having the amino acid sequence of SEQ ID NO: 77;
   (d) a light chain CDR1 having the amino acid sequence of SEQ ID NO: 78;
   (e) a light chain CDR2 having the amino acid sequence of SEQ ID NO: 79; and
   (f) a light chain CDR3 having the amino acid sequence of SEQ ID NO: 80, or
a conservative substitution variant of one or more of (a)-(f), or
an antibody, antibody reagent, or antigen-binding portion thereof comprising the heavy chain variable region sequence of SEQ ID NO: 9, or
an antibody, antibody reagent, or antigen-binding portion thereof comprising the light chain variable region sequence of SEQ ID NO: 10, or
an antibody, antibody reagent, or antigen-binding portion thereof comprising the heavy chain variable region sequence of SEQ ID NO: 25, or
an antibody, antibody reagent, or antigen-binding portion thereof comprising the light chain variable region sequence of SEQ ID NO: 26, or
an antibody, antibody reagent, or antigen-binding portion thereof comprising the heavy chain variable region sequence of SEQ ID NO: 33, or
an antibody, antibody reagent, or antigen-binding portion thereof comprising the light chain variable region sequence of SEQ ID NO: 34, or
an antibody, antibody reagent, or antigen-binding portion thereof comprising the heavy chain variable region sequence of SEQ ID NO: 41, or
an antibody, antibody reagent, or antigen-binding portion thereof comprising the light chain variable region sequence of SEQ ID NO: 42, or
an antibody, antibody reagent, or antigen-binding portion thereof comprising the heavy chain variable region sequence of SEQ ID NO: 49, or
an antibody, antibody reagent, or antigen-binding portion thereof comprising the light chain variable region sequence of SEQ ID NO: 50, or
an antibody, antibody reagent, or antigen-binding portion thereof comprising the heavy chain variable region sequence of SEQ ID NO: 57, or
an antibody, antibody reagent, or antigen-binding portion thereof comprising the light chain variable region sequence of SEQ ID NO: 58, or
an antibody, antibody reagent, or antigen-binding portion thereof comprising the heavy chain variable region sequence of SEQ ID NO: 65, or
an antibody, antibody reagent, or antigen-binding portion thereof comprising the light chain variable region sequence of SEQ ID NO: 66, or
an antibody, antibody reagent, or antigen-binding portion thereof comprising the heavy chain variable region sequence of SEQ ID NO: 73, or
an antibody, antibody reagent, or antigen-binding portion thereof comprising the light chain variable region sequence of SEQ ID NO: 74; and
(ii) an immunosuppressive agent.

18. The composition, kit, or combination of paragraph 17, wherein the antibody, antibody reagent, or antigen-binding portion thereof is conjugated to the immunosuppressive agent.

19. A nucleic acid sequence encoding the antibody, antibody reagent, antigen-binding fragment thereof, or CAR of any one of paragraphs 1-16.

20. A cell comprising the antibody, antibody reagent, antigen-binding fragment thereof, or CAR of any one of paragraphs 1-16 or the nucleic acid sequence of paragraph 19.

21. A pharmaceutical composition comprising the antibody, antibody reagent, antigen-binding fragment thereof, or CAR of any one of paragraphs 1-16; or the composition, kit, or combination of any one of paragraphs 17-18; or the cell of paragraph 20, and a pharmaceutically acceptable carrier.

22. A solid support comprising the antibody, antibody reagent, antigen-binding fragment thereof, or CAR of any one of paragraphs 1-16.

23. The solid support of paragraph 22, wherein the antibody, antibody reagent or antigen-binding fragment thereof is detectably labeled.

24. The solid support of any one of paragraphs 22-23, wherein the solid support comprises a particle, a bead, a polymer, or a substrate.

25. A kit for the detection of PD1 polypeptide in a sample, the kit comprising at least a first antibody, antibody reagent, antigen-binding fragment thereof, or CAR of any one of paragraphs 1-16 immobilized on a solid support and comprising a detectable label.

26. A molecular complex comprising at least one antibody, antibody reagent, antigen-binding fragment thereof, or CAR of any one of paragraphs 1-16 bound to an PD1 polypeptide.

27. A method of treating an autoimmune disorder or an auto-inflammatory disorder in a subject in need thereof, the method comprising administering
(i) the antibody, antibody reagent, antigen-binding portion thereof, or CAR of any one of paragraphs 1-16 or an antibody, antibody reagent, antigen-binding portion thereof, or CAR comprising at least one heavy or light chain complementarity determining region (CDR) selected from the group consisting of:
   (a) a heavy chain CDR1 having the amino acid sequence of SEQ ID NO: 11;
   (b) a heavy chain CDR2 having the amino acid sequence of SEQ ID NO: 12;
   (c) a heavy chain CDR3 having the amino acid sequence of SEQ ID NO: 13;
   (d) a light chain CDR1 having the amino acid sequence of SEQ ID NO: 14;
   (e) a light chain CDR2 having the amino acid sequence of SEQ ID NO: 15; and
   (f) a light chain CDR3 having the amino acid sequence of SEQ ID NO: 16, or
selected from the group consisting of:
   (a) a heavy chain CDR1 having the amino acid sequence of SEQ ID NO: 27;
   (b) a heavy chain CDR2 having the amino acid sequence of SEQ ID NO: 28;
   (c) a heavy chain CDR3 having the amino acid sequence of SEQ ID NO: 29;
   (d) a light chain CDR1 having the amino acid sequence of SEQ ID NO: 30;
   (e) a light chain CDR2 having the amino acid sequence of SEQ ID NO: 31; and
   (f) a light chain CDR3 having the amino acid sequence of SEQ ID NO: 32, or
selected from the group consisting of:
   (a) a heavy chain CDR1 having the amino acid sequence of SEQ ID NO: 35;
   (b) a heavy chain CDR2 having the amino acid sequence of SEQ ID NO: 36;
   (c) a heavy chain CDR3 having the amino acid sequence of SEQ ID NO: 37;
   (d) a light chain CDR1 having the amino acid sequence of SEQ ID NO: 38;
   (e) a light chain CDR2 having the amino acid sequence of SEQ ID NO: 39; and
   (f) a light chain CDR3 having the amino acid sequence of SEQ ID NO: 40, or
selected from the group consisting of:
   (a) a heavy chain CDR1 having the amino acid sequence of SEQ ID NO: 43;
   (b) a heavy chain CDR2 having the amino acid sequence of SEQ ID NO: 44;
   (c) a heavy chain CDR3 having the amino acid sequence of SEQ ID NO: 45;
   (d) a light chain CDR1 having the amino acid sequence of SEQ ID NO: 46;
   (e) a light chain CDR2 having the amino acid sequence of SEQ ID NO: 47; and
   (f) a light chain CDR3 having the amino acid sequence of SEQ ID NO: 48, or
selected from the group consisting of:
   (a) a heavy chain CDR1 having the amino acid sequence of SEQ ID NO: 51;
   (b) a heavy chain CDR2 having the amino acid sequence of SEQ ID NO: 52;
   (c) a heavy chain CDR3 having the amino acid sequence of SEQ ID NO: 53;
   (d) a light chain CDR1 having the amino acid sequence of SEQ ID NO: 54;
   (e) a light chain CDR2 having the amino acid sequence of SEQ ID NO: 55; and
   (f) a light chain CDR3 having the amino acid sequence of SEQ ID NO: 56, or
selected from the group consisting of:
   (a) a heavy chain CDR1 having the amino acid sequence of SEQ ID NO: 59;
   (b) a heavy chain CDR2 having the amino acid sequence of SEQ ID NO: 60;
   (c) a heavy chain CDR3 having the amino acid sequence of SEQ ID NO: 61;
   (d) a light chain CDR1 having the amino acid sequence of SEQ ID NO: 62;
   (e) a light chain CDR2 having the amino acid sequence of SEQ ID NO: 63; and
   (f) a light chain CDR3 having the amino acid sequence of SEQ ID NO: 64, or
selected from the group consisting of:
   (a) a heavy chain CDR1 having the amino acid sequence of SEQ ID NO: 67;
   (b) a heavy chain CDR2 having the amino acid sequence of SEQ ID NO: 68;
   (c) a heavy chain CDR3 having the amino acid sequence of SEQ ID NO: 69;
   (d) a light chain CDR1 having the amino acid sequence of SEQ ID NO: 70;
   (e) a light chain CDR2 having the amino acid sequence of SEQ ID NO: 71; and
   (f) a light chain CDR3 having the amino acid sequence of SEQ ID NO: 72, or
selected from the group consisting of:
   (a) a heavy chain CDR1 having the amino acid sequence of SEQ ID NO: 75;
   (b) a heavy chain CDR2 having the amino acid sequence of SEQ ID NO: 76;
   (c) a heavy chain CDR3 having the amino acid sequence of SEQ ID NO: 77;
   (d) a light chain CDR1 having the amino acid sequence of SEQ ID NO: 78;
   (e) a light chain CDR2 having the amino acid sequence of SEQ ID NO: 79; and
   (f) a light chain CDR3 having the amino acid sequence of SEQ ID NO: 80, or
a conservative substitution variant of one or more of (a)-(f), or
an antibody, antibody reagent, or antigen-binding portion thereof comprising the heavy chain variable region sequence of SEQ ID NO: 9, or
an antibody, antibody reagent, or antigen-binding portion thereof comprising the light chain variable region sequence of SEQ ID NO: 10, or
an antibody, antibody reagent, or antigen-binding portion thereof comprising the heavy chain variable region sequence of SEQ ID NO: 25, or
an antibody, antibody reagent, or antigen-binding portion thereof comprising the light chain variable region sequence of SEQ ID NO: 26, or
an antibody, antibody reagent, or antigen-binding portion thereof comprising the heavy chain variable region sequence of SEQ ID NO: 33, or
an antibody, antibody reagent, or antigen-binding portion thereof comprising the light chain variable region sequence of SEQ ID NO: 34, or
an antibody, antibody reagent, or antigen-binding portion thereof comprising the heavy chain variable region sequence of SEQ ID NO: 41, or
an antibody, antibody reagent, or antigen-binding portion thereof comprising the light chain variable region sequence of SEQ ID NO: 42, or
an antibody, antibody reagent, or antigen-binding portion thereof comprising the heavy chain variable region sequence of SEQ ID NO: 49, or
an antibody, antibody reagent, or antigen-binding portion thereof comprising the light chain variable region sequence of SEQ ID NO: 50, or
an antibody, antibody reagent, or antigen-binding portion thereof comprising the heavy chain variable region sequence of SEQ ID NO: 57, or
an antibody, antibody reagent, or antigen-binding portion thereof comprising the light chain variable region sequence of SEQ ID NO: 58, or
an antibody, antibody reagent, or antigen-binding portion thereof comprising the heavy chain variable region sequence of SEQ ID NO: 65, or
an antibody, antibody reagent, or antigen-binding portion thereof comprising the light chain variable region sequence of SEQ ID NO: 66, or
an antibody, antibody reagent, or antigen-binding portion thereof comprising the heavy chain variable region sequence of SEQ ID NO: 73, or
an antibody, antibody reagent, or antigen-binding portion thereof comprising the light chain variable region sequence of SEQ ID NO: 74; or
(ii) the composition kit, or combination of any one of paragraphs 17-18; or
(iii) the cell of paragraph 20, to the subject.

28. The method of paragraph 27, wherein the autoimmune disorder or the autoinflammatory disorder is a T-cell mediated disorder.

29. The method of any one of paragraph 27-28, wherein the autoimmune disorder or the autoinflammatory disorder is selected from the group consisting of:
type-1 diabetes, rheumatoid arthritis, multiple sclerosis, celiac disease, Rasmussen's encephalitis, hypothyroidism, Addison's disease, and amyotrophic lateral sclerosis (ALS).

30. The antibody, antibody reagent, antigen-binding fragment thereof, or CAR of any one of paragraphs 1-16 or an antibody, antibody reagent, antigen-binding portion thereof, or CAR comprising at least one heavy or light chain complementarity determining region (CDR) selected from the group consisting of:
(a) a heavy chain CDR1 having the amino acid sequence of SEQ ID NO: 11;
(b) a heavy chain CDR2 having the amino acid sequence of SEQ ID NO: 12;
(c) a heavy chain CDR3 having the amino acid sequence of SEQ ID NO: 13;
(d) a light chain CDR1 having the amino acid sequence of SEQ ID NO: 14;
(e) a light chain CDR2 having the amino acid sequence of SEQ ID NO: 15; and
(f) a light chain CDR3 having the amino acid sequence of SEQ ID NO: 16, or
   selected from the group consisting of:
(a) a heavy chain CDR1 having the amino acid sequence of SEQ ID NO: 27;
(b) a heavy chain CDR2 having the amino acid sequence of SEQ ID NO: 28;
(c) a heavy chain CDR3 having the amino acid sequence of SEQ ID NO: 29;
(d) a light chain CDR1 having the amino acid sequence of SEQ ID NO: 30;
(e) a light chain CDR2 having the amino acid sequence of SEQ ID NO: 31; and
(f) a light chain CDR3 having the amino acid sequence of SEQ ID NO: 32, or
   selected from the group consisting of:
(a) a heavy chain CDR1 having the amino acid sequence of SEQ ID NO: 35;
(b) a heavy chain CDR2 having the amino acid sequence of SEQ ID NO: 36;
(c) a heavy chain CDR3 having the amino acid sequence of SEQ ID NO: 37;
(d) a light chain CDR1 having the amino acid sequence of SEQ ID NO: 38;
(e) a light chain CDR2 having the amino acid sequence of SEQ ID NO: 39; and
(f) a light chain CDR3 having the amino acid sequence of SEQ ID NO: 40, or
   selected from the group consisting of:
(a) a heavy chain CDR1 having the amino acid sequence of SEQ ID NO: 43;
(b) a heavy chain CDR2 having the amino acid sequence of SEQ ID NO: 44;
(c) a heavy chain CDR3 having the amino acid sequence of SEQ ID NO: 45;
(d) a light chain CDR1 having the amino acid sequence of SEQ ID NO: 46;
(e) a light chain CDR2 having the amino acid sequence of SEQ ID NO: 47; and
(f) a light chain CDR3 having the amino acid sequence of SEQ ID NO: 48, or
   selected from the group consisting of:
(a) a heavy chain CDR1 having the amino acid sequence of SEQ ID NO: 51;
(b) a heavy chain CDR2 having the amino acid sequence of SEQ ID NO: 52;
(c) a heavy chain CDR3 having the amino acid sequence of SEQ ID NO: 53;
(d) a light chain CDR1 having the amino acid sequence of SEQ ID NO: 54;
(e) a light chain CDR2 having the amino acid sequence of SEQ ID NO: 55; and
(f) a light chain CDR3 having the amino acid sequence of SEQ ID NO: 56, or
   selected from the group consisting of:
(a) a heavy chain CDR1 having the amino acid sequence of SEQ ID NO: 59;
(b) a heavy chain CDR2 having the amino acid sequence of SEQ ID NO: 60;
(c) a heavy chain CDR3 having the amino acid sequence of SEQ ID NO: 61;
(d) a light chain CDR1 having the amino acid sequence of SEQ ID NO: 62;
(e) a light chain CDR2 having the amino acid sequence of SEQ ID NO: 63; and
(f) a light chain CDR3 having the amino acid sequence of SEQ ID NO: 64, or
   selected from the group consisting of:
(a) a heavy chain CDR1 having the amino acid sequence of SEQ ID NO: 67;
(b) a heavy chain CDR2 having the amino acid sequence of SEQ ID NO: 68;
(c) a heavy chain CDR3 having the amino acid sequence of SEQ ID NO: 69;
(d) a light chain CDR1 having the amino acid sequence of SEQ ID NO: 70;
(e) a light chain CDR2 having the amino acid sequence of SEQ ID NO: 71; and
(f) a light chain CDR3 having the amino acid sequence of SEQ ID NO: 72, or
   selected from the group consisting of:
(a) a heavy chain CDR1 having the amino acid sequence of SEQ ID NO: 75;
(b) a heavy chain CDR2 having the amino acid sequence of SEQ ID NO: 76;
(c) a heavy chain CDR3 having the amino acid sequence of SEQ ID NO: 77;
(d) a light chain CDR1 having the amino acid sequence of SEQ ID NO: 78;
(e) a light chain CDR2 having the amino acid sequence of SEQ ID NO: 79; and
(f) a light chain CDR3 having the amino acid sequence of SEQ ID NO: 80, or
a conservative substitution variant of one or more of (a)-(f); or
an antibody, antibody reagent, or antigen-binding portion thereof comprising the heavy chain variable region sequence of SEQ ID NO: 9, or
an antibody, antibody reagent, or antigen-binding portion thereof comprising the light chain variable region sequence of SEQ ID NO: 10, or
an antibody, antibody reagent, or antigen-binding portion thereof comprising the heavy chain variable region sequence of SEQ ID NO: 25, or
   an antibody, antibody reagent, or antigen-binding portion thereof comprising the light chain variable region sequence of SEQ ID NO: 26, or
an antibody, antibody reagent, or antigen-binding portion thereof comprising the heavy chain variable region sequence of SEQ ID NO: 33, or
an antibody, antibody reagent, or antigen-binding portion thereof comprising the light chain variable region sequence of SEQ ID NO: 34, or
an antibody, antibody reagent, or antigen-binding portion thereof comprising the heavy chain variable region sequence of SEQ ID NO: 41, or
an antibody, antibody reagent, or antigen-binding portion thereof comprising the light chain variable region sequence of SEQ ID NO: 42, or
an antibody, antibody reagent, or antigen-binding portion thereof comprising the heavy chain variable region sequence of SEQ ID NO: 49, or
an antibody, antibody reagent, or antigen-binding portion thereof comprising the light chain variable region sequence of SEQ ID NO: 50, or
an antibody, antibody reagent, or antigen-binding portion thereof comprising the heavy chain variable region sequence of SEQ ID NO: 57, or
an antibody, antibody reagent, or antigen-binding portion thereof comprising the light chain variable region sequence of SEQ ID NO: 58, or
an antibody, antibody reagent, or antigen-binding portion thereof comprising the heavy chain variable region sequence of SEQ ID NO: 65, or
an antibody, antibody reagent, or antigen-binding portion thereof comprising the light chain variable region sequence of SEQ ID NO: 66, or
an antibody, antibody reagent, or antigen-binding portion thereof comprising the heavy chain variable region sequence of SEQ ID NO: 73, or
an antibody, antibody reagent, or antigen-binding portion thereof comprising the light chain variable region sequence of SEQ ID NO: 74; or
the composition, kit, or combination of any one of paragraphs 17-18; or the cell of paragraph 20, for use in a method of treating an autoimmune disorder or an auto-inflammatory disorder in a subject in need thereof.

31. The composition of paragraph 30 wherein the autoimmune disorder or the autoinflammatory disorder is a T-cell mediated disorder.

32. The composition of any one of paragraphs 30-31, wherein the autoimmune disorder or the autoinflammatory disorder is selected from the group consisting of:
type-1 diabetes, rheumatoid arthritis, multiple sclerosis, celiac disease, Rasmussen's encephalitis, hypothyroidism, Addison's disease, and amyotrophic lateral sclerosis (ALS).

33. A method of suppressing an immune response or an inflammatory response in a subject in need thereof, the method comprising administering
(i) the antibody, antibody reagent, antigen-binding portion thereof, or CAR of any one of paragraphs 1-16 or an antibody, antibody reagent, antigen-binding portion thereof, or CAR comprising at least one heavy or light chain complementarity determining region (CDR) selected from the group consisting of:
   (a) a heavy chain CDR1 having the amino acid sequence of SEQ ID NO: 11;
   (b) a heavy chain CDR2 having the amino acid sequence of SEQ ID NO: 12;
   (c) a heavy chain CDR3 having the amino acid sequence of SEQ ID NO: 13;
   (d) a light chain CDR1 having the amino acid sequence of SEQ ID NO: 14;
   (e) a light chain CDR2 having the amino acid sequence of SEQ ID NO: 15; and
   (f) a light chain CDR3 having the amino acid sequence of SEQ ID NO: 16, or
selected from the group consisting of:
   (a) a heavy chain CDR1 having the amino acid sequence of SEQ ID NO: 27;
   (b) a heavy chain CDR2 having the amino acid sequence of SEQ ID NO: 28;
   (c) a heavy chain CDR3 having the amino acid sequence of SEQ ID NO: 29;
   (d) a light chain CDR1 having the amino acid sequence of SEQ ID NO: 30;
   (e) a light chain CDR2 having the amino acid sequence of SEQ ID NO: 31; and
   (f) a light chain CDR3 having the amino acid sequence of SEQ ID NO: 32, or
selected from the group consisting of:
   (a) a heavy chain CDR1 having the amino acid sequence of SEQ ID NO: 35;
   (b) a heavy chain CDR2 having the amino acid sequence of SEQ ID NO: 36;
   (c) a heavy chain CDR3 having the amino acid sequence of SEQ ID NO: 37;
   (d) a light chain CDR1 having the amino acid sequence of SEQ ID NO: 38;
   (e) a light chain CDR2 having the amino acid sequence of SEQ ID NO: 39; and
   (f) a light chain CDR3 having the amino acid sequence of SEQ ID NO: 40, or
selected from the group consisting of:
   (a) a heavy chain CDR1 having the amino acid sequence of SEQ ID NO: 43;
   (b) a heavy chain CDR2 having the amino acid sequence of SEQ ID NO: 44;
   (c) a heavy chain CDR3 having the amino acid sequence of SEQ ID NO: 45;
   (d) a light chain CDR1 having the amino acid sequence of SEQ ID NO: 46;
   (e) a light chain CDR2 having the amino acid sequence of SEQ ID NO: 47; and
   (f) a light chain CDR3 having the amino acid sequence of SEQ ID NO: 48, or
selected from the group consisting of:
   (a) a heavy chain CDR1 having the amino acid sequence of SEQ ID NO: 51;
   (b) a heavy chain CDR2 having the amino acid sequence of SEQ ID NO: 52;
   (c) a heavy chain CDR3 having the amino acid sequence of SEQ ID NO: 53;
   (d) a light chain CDR1 having the amino acid sequence of SEQ ID NO: 54;
   (e) a light chain CDR2 having the amino acid sequence of SEQ ID NO: 55; and
   (f) a light chain CDR3 having the amino acid sequence of SEQ ID NO: 56, or
selected from the group consisting of:
   (a) a heavy chain CDR1 having the amino acid sequence of SEQ ID NO: 59;
   (b) a heavy chain CDR2 having the amino acid sequence of SEQ ID NO: 60;
   (c) a heavy chain CDR3 having the amino acid sequence of SEQ ID NO: 61;
   (d) a light chain CDR1 having the amino acid sequence of SEQ ID NO: 62;
   (e) a light chain CDR2 having the amino acid sequence of SEQ ID NO: 63; and
   (f) a light chain CDR3 having the amino acid sequence of SEQ ID NO: 64, or
selected from the group consisting of:
   (a) a heavy chain CDR1 having the amino acid sequence of SEQ ID NO: 67;
   (b) a heavy chain CDR2 having the amino acid sequence of SEQ ID NO: 68;
   (c) a heavy chain CDR3 having the amino acid sequence of SEQ ID NO: 69;
   (d) a light chain CDR1 having the amino acid sequence of SEQ ID NO: 70;
   (e) a light chain CDR2 having the amino acid sequence of SEQ ID NO: 71; and
   (f) a light chain CDR3 having the amino acid sequence of SEQ ID NO: 72, or
selected from the group consisting of:
   (a) a heavy chain CDR1 having the amino acid sequence of SEQ ID NO: 75;
   (b) a heavy chain CDR2 having the amino acid sequence of SEQ ID NO: 76;
   (c) a heavy chain CDR3 having the amino acid sequence of SEQ ID NO: 77;
   (d) a light chain CDR1 having the amino acid sequence of SEQ ID NO: 78;
   (e) a light chain CDR2 having the amino acid sequence of SEQ ID NO: 79; and
   (f) a light chain CDR3 having the amino acid sequence of SEQ ID NO: 80, or
a conservative substitution variant of one or more of (a)-(f), or
an antibody, antibody reagent, or antigen-binding portion thereof comprising the heavy chain variable region sequence of SEQ ID NO: 9, or
an antibody, antibody reagent, or antigen-binding portion thereof comprising the light chain variable region sequence of SEQ ID NO: 10, or
an antibody, antibody reagent, or antigen-binding portion thereof comprising the heavy chain variable region sequence of SEQ ID NO: 25, or
an antibody, antibody reagent, or antigen-binding portion thereof comprising the light chain variable region sequence of SEQ ID NO: 26, or
an antibody, antibody reagent, or antigen-binding portion thereof comprising the heavy chain variable region sequence of SEQ ID NO: 33, or
an antibody, antibody reagent, or antigen-binding portion thereof comprising the light chain variable region sequence of SEQ ID NO: 34, or
an antibody, antibody reagent, or antigen-binding portion thereof comprising the heavy chain variable region sequence of SEQ ID NO: 41, or
an antibody, antibody reagent, or antigen-binding portion thereof comprising the light chain variable region sequence of SEQ ID NO: 42, or
an antibody, antibody reagent, or antigen-binding portion thereof comprising the heavy chain variable region sequence of SEQ ID NO: 49, or
an antibody, antibody reagent, or antigen-binding portion thereof comprising the light chain variable region sequence of SEQ ID NO: 50, or
an antibody, antibody reagent, or antigen-binding portion thereof comprising the heavy chain variable region sequence of SEQ ID NO: 57, or
an antibody, antibody reagent, or antigen-binding portion thereof comprising the light chain variable region sequence of SEQ ID NO: 58, or
an antibody, antibody reagent, or antigen-binding portion thereof comprising the heavy chain variable region sequence of SEQ ID NO: 65, or
an antibody, antibody reagent, or antigen-binding portion thereof comprising the light chain variable region sequence of SEQ ID NO: 66, or
an antibody, antibody reagent, or antigen-binding portion thereof comprising the heavy chain variable region sequence of SEQ ID NO: 73, or
an antibody, antibody reagent, or antigen-binding portion thereof comprising the light chain variable region sequence of SEQ ID NO: 74; or
(ii) the composition kit, or combination of any one of paragraphs 17-18; or
(iii) the cell of paragraph 20, to the subject.

34. The method of paragraph 33, wherein the immune response or the inflammatory response is a T-cell mediated response.

35. The method of any one of paragraph 33-34, wherein the immune response or the inflammatory response is associated with the group consisting of:
type-1 diabetes, rheumatoid arthritis, multiple sclerosis, celiac disease, Rasmussen's encephalitis, hypothyroidism, Addison's disease, and amyotrophic lateral sclerosis (ALS).

36. The antibody, antibody reagent, antigen-binding fragment thereof, or CAR of any one of paragraphs 1-16 or an antibody, antibody reagent, antigen-binding portion thereof, or CAR comprising at least one heavy or light chain complementarity determining region (CDR) selected from the group consisting of:
(a) a heavy chain CDR1 having the amino acid sequence of SEQ ID NO: 11;
(b) a heavy chain CDR2 having the amino acid sequence of SEQ ID NO: 12;
(c) a heavy chain CDR3 having the amino acid sequence of SEQ ID NO: 13;
(d) a light chain CDR1 having the amino acid sequence of SEQ ID NO: 14;
(e) a light chain CDR2 having the amino acid sequence of SEQ ID NO: 15; and
(f) a light chain CDR3 having the amino acid sequence of SEQ ID NO: 16, or
   selected from the group consisting of:
(a) a heavy chain CDR1 having the amino acid sequence of SEQ ID NO: 27;
(b) a heavy chain CDR2 having the amino acid sequence of SEQ ID NO: 28;
(c) a heavy chain CDR3 having the amino acid sequence of SEQ ID NO: 29;
(d) a light chain CDR1 having the amino acid sequence of SEQ ID NO: 30;
(e) a light chain CDR2 having the amino acid sequence of SEQ ID NO: 31; and
(f) a light chain CDR3 having the amino acid sequence of SEQ ID NO: 32, or
   selected from the group consisting of:
(a) a heavy chain CDR1 having the amino acid sequence of SEQ ID NO: 35;
(b) a heavy chain CDR2 having the amino acid sequence of SEQ ID NO: 36;
(c) a heavy chain CDR3 having the amino acid sequence of SEQ ID NO: 37;
(d) a light chain CDR1 having the amino acid sequence of SEQ ID NO: 38;
(e) a light chain CDR2 having the amino acid sequence of SEQ ID NO: 39; and
(f) a light chain CDR3 having the amino acid sequence of SEQ ID NO: 40, or
   selected from the group consisting of:
(a) a heavy chain CDR1 having the amino acid sequence of SEQ ID NO: 43;
(b) a heavy chain CDR2 having the amino acid sequence of SEQ ID NO: 44;
(c) a heavy chain CDR3 having the amino acid sequence of SEQ ID NO: 45;
(d) a light chain CDR1 having the amino acid sequence of SEQ ID NO: 46;
(e) a light chain CDR2 having the amino acid sequence of SEQ ID NO: 47; and
(f) a light chain CDR3 having the amino acid sequence of SEQ ID NO: 48, or
   selected from the group consisting of:
(a) a heavy chain CDR1 having the amino acid sequence of SEQ ID NO: 51;
(b) a heavy chain CDR2 having the amino acid sequence of SEQ ID NO: 52;
(c) a heavy chain CDR3 having the amino acid sequence of SEQ ID NO: 53;
(d) a light chain CDR1 having the amino acid sequence of SEQ ID NO: 54;
(e) a light chain CDR2 having the amino acid sequence of SEQ ID NO: 55; and
(f) a light chain CDR3 having the amino acid sequence of SEQ ID NO: 56, or
   selected from the group consisting of:
(a) a heavy chain CDR1 having the amino acid sequence of SEQ ID NO: 59;
(b) a heavy chain CDR2 having the amino acid sequence of SEQ ID NO: 60;
(c) a heavy chain CDR3 having the amino acid sequence of SEQ ID NO: 61;
(d) a light chain CDR1 having the amino acid sequence of SEQ ID NO: 62;
(e) a light chain CDR2 having the amino acid sequence of SEQ ID NO: 63; and
(f) a light chain CDR3 having the amino acid sequence of SEQ ID NO: 64, or
   selected from the group consisting of:
(a) a heavy chain CDR1 having the amino acid sequence of SEQ ID NO: 67;
(b) a heavy chain CDR2 having the amino acid sequence of SEQ ID NO: 68;
(c) a heavy chain CDR3 having the amino acid sequence of SEQ ID NO: 69;
(d) a light chain CDR1 having the amino acid sequence of SEQ ID NO: 70;
(e) a light chain CDR2 having the amino acid sequence of SEQ ID NO: 71; and
(f) a light chain CDR3 having the amino acid sequence of SEQ ID NO: 72, or
   selected from the group consisting of:
(a) a heavy chain CDR1 having the amino acid sequence of SEQ ID NO: 75;
(b) a heavy chain CDR2 having the amino acid sequence of SEQ ID NO: 76;
(c) a heavy chain CDR3 having the amino acid sequence of SEQ ID NO: 77;
(d) a light chain CDR1 having the amino acid sequence of SEQ ID NO: 78;
(e) a light chain CDR2 having the amino acid sequence of SEQ ID NO: 79; and
(f) a light chain CDR3 having the amino acid sequence of SEQ ID NO: 80, or
a conservative substitution variant of one or more of (a)-(f); or
an antibody, antibody reagent, or antigen-binding portion thereof comprising the heavy chain variable region sequence of SEQ ID NO: 9, or
an antibody, antibody reagent, or antigen-binding portion thereof comprising the light chain variable region sequence of SEQ ID NO: 10, or
an antibody, antibody reagent, or antigen-binding portion thereof comprising the heavy chain variable region sequence of SEQ ID NO: 25, or
an antibody, antibody reagent, or antigen-binding portion thereof comprising the light chain variable region sequence of SEQ ID NO: 26, or
an antibody, antibody reagent, or antigen-binding portion thereof comprising the heavy chain variable region sequence of SEQ ID NO: 33, or
an antibody, antibody reagent, or antigen-binding portion thereof comprising the light chain variable region sequence of SEQ ID NO: 34, or
an antibody, antibody reagent, or antigen-binding portion thereof comprising the heavy chain variable region sequence of SEQ ID NO: 41, or
an antibody, antibody reagent, or antigen-binding portion thereof comprising the light chain variable region sequence of SEQ ID NO: 42, or
an antibody, antibody reagent, or antigen-binding portion thereof comprising the heavy chain variable region sequence of SEQ ID NO: 49, or
an antibody, antibody reagent, or antigen-binding portion thereof comprising the light chain variable region sequence of SEQ ID NO: 50, or
an antibody, antibody reagent, or antigen-binding portion thereof comprising the heavy chain variable region sequence of SEQ ID NO: 57, or
an antibody, antibody reagent, or antigen-binding portion thereof comprising the light chain variable region sequence of SEQ ID NO: 58, or
an antibody, antibody reagent, or antigen-binding portion thereof comprising the heavy chain variable region sequence of SEQ ID NO: 65, or
an antibody, antibody reagent, or antigen-binding portion thereof comprising the light chain variable region sequence of SEQ ID NO: 66, or
an antibody, antibody reagent, or antigen-binding portion thereof comprising the heavy chain variable region sequence of SEQ ID NO: 73, or
an antibody, antibody reagent, or antigen-binding portion thereof comprising the light chain variable region sequence of SEQ ID NO: 74; or
the composition, kit, or combination of any one of paragraphs 17-18; or the cell of paragraph 20, for use in a method suppressing an immune response or an inflammatory response in a subject in need thereof.

37. The composition of paragraph 36 wherein the immune response or the inflammatory response is a T-cell mediated response.

38. The composition of any one of paragraphs 36-37, wherein the immune response or the inflammatory response is associated with the group consisting of:
type-1 diabetes, rheumatoid arthritis, multiple sclerosis, celiac disease, Rasmussen's encephalitis, hypothyroidism, Addison's disease, and amyotrophic lateral sclerosis (ALS).

39. An antibody, antibody reagent, antigen-binding fragment thereof, or chimaeric antigen receptor (CAR), that specifically binds an PD1 polypeptide, said antibody reagent, antigen-binding portion thereof, or CAR comprising at least one heavy or light chain complementarity determining region (CDR) selected from the group consisting of:
(a) a heavy chain CDR1 having the amino acid sequence of SEQ ID NO: 83;
(b) a heavy chain CDR2 having the amino acid sequence of SEQ ID NO: 84;
(c) a heavy chain CDR3 having the amino acid sequence of SEQ ID NO: 85;
(d) a light chain CDR1 having the amino acid sequence of SEQ ID NO: 86;
(e) a light chain CDR2 having the amino acid sequence of SEQ ID NO: 87; and
(f) a light chain CDR3 having the amino acid sequence of SEQ ID NO: 88, or
   a conservative substitution variant of one or more of (a)-(f).

40. The antibody, antibody reagent, antigen-binding portion thereof, or CAR of paragraph 39, which comprises heavy chain CDRs having the amino acid sequences of SEQ ID NOs: 83-85, or a conservative substitution variant of such amino acid sequence.

41. The antibody, antibody reagent, antigen-binding portion thereof, or CAR of any of paragraphs 39-40, which comprises light chain CDRs having the amino acid sequences of SEQ ID NOs: 86-88 or a conservative substitution variant of such amino acid sequence.

42. The antibody, antibody reagent, antigen-binding portion thereof, or CAR of any of paragraphs 39-41, which comprises: heavy chain CDRs having the amino acid sequences of SEQ ID NOs: 83-85 and light chain CDRs having the amino acid sequences of SEQ ID NOs: 86-88 or a conservative substitution variant of such amino acid sequence.

43. A first antibody, antibody reagent, antigen-binding portion thereof, or CAR that specifically binds an PD1 polypeptide, and can compete for binding of PD1 with a second antibody comprising: heavy chain CDRs having the amino acid sequences of SEQ ID NOs: 83-85 and light chain CDRs having the amino acid sequences of SEQ ID NOs: 86-88 or a conservative substitution variant of such amino acid sequence.

44. The first antibody, antibody reagent, antigen-binding fragment thereof, or chimaeric antigen receptor (CAR) of paragraph 43, comprising at least one heavy or light chain complementarity determining region (CDR) selected from the group consisting of:
(a) a heavy chain CDR1 having the amino acid sequence of SEQ ID NO: 83;
(b) a heavy chain CDR2 having the amino acid sequence of SEQ ID NO: 84;
(c) a heavy chain CDR3 having the amino acid sequence of SEQ ID NO: 85;
(d) a light chain CDR1 having the amino acid sequence of SEQ ID NO: 86;
(e) a light chain CDR2 having the amino acid sequence of SEQ ID NO: 87; and
(f) a light chain CDR3 having the amino acid sequence of SEQ ID NO: 88, or
   a conservative substitution variant of one or more of (a)-(f).

45. The antibody, antibody reagent, antigen-binding portion thereof, or CAR of paragraph 43 or 44, which comprises heavy chain CDRs having the amino acid sequences of SEQ ID NOs: 83-85, or a conservative substitution variant of such amino acid sequence.

46. The antibody, antibody reagent, antigen-binding portion thereof, or CAR of any of paragraphs 43-45, which comprises light chain CDRs having the amino acid sequences of SEQ ID NOs: 86-88, or a conservative substitution variant of such amino acid sequence.

47. The antibody, antibody reagent, antigen-binding portion thereof, or CAR of any of paragraphs 43-46, which comprises: heavy chain CDRs having the amino acid sequences of SEQ ID NOs: 83-85 and light chain CDRs having the amino acid sequences of SEQ ID NOs: 86-88 or a conservative substitution variant of such amino acid sequence.

48. The antibody, antibody reagent, antigen-binding portion thereof, or CAR of any of paragraphs 39-47, comprising the heavy chain sequence of SEQ ID NO: 81.

49. The antibody, antibody reagent, antigen-binding portion thereof, or CAR of any of paragraphs 39-48, comprising the light chain sequence of SEQ ID NO: 82.

50. The antibody, antibody reagent, antigen-binding portion thereof, or CAR of any of paragraphs 39-49, comprising: the heavy chain variable region sequence of SEQ ID NO: 81 and the light chain variable region sequence of SEQ ID NO: 82.

51. The antibody, antibody reagent, antigen-binding portion thereof, or CAR of any of paragraphs 39-50, further comprising a conservative substitution in a sequence not comprised by a CDR.

52. The antibody, antibody reagent, antigen-binding portion thereof, or CAR of any of paragraphs 39-51, wherein the antibody reagent or antigen-binding fragment thereof is fully human or fully humanized.

53. The antibody, antibody reagent, antigen-binding portion thereof, or CAR of any of paragraphs 39-52, wherein the antibody reagent or antigen-binding fragment thereof is fully humanized except for the CDR sequences.

54. The antibody, antibody reagent, antigen-binding portion thereof, or CAR of any of paragraphs 39-53, wherein the reagent or fragment is selected from the group consisting of:
an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, and a bispecific antibody.

55. A composition comprising the antibody, antibody reagent, antigen-binding portion thereof, or CAR of any one of paragraphs 39-54, and a chemotherapeutic agent.

56. The composition of paragraph 55, wherein the antibody, antibody reagent, or antigen-binding portion thereof is conjugated to the chemotherapeutic agent.

57. A nucleic acid sequence encoding the antibody, antibody reagent, antigen-binding fragment thereof, or CAR of any of paragraphs 39-54.

58. A cell comprising the antibody, antibody reagent, antigen-binding fragment thereof, or CAR of any of paragraphs 49-54 or the nucleic acid sequence of paragraph 57.

59. A pharmaceutical composition comprising the antibody, antibody reagent, antigen-binding fragment thereof, or CAR of any of paragraphs 39-54; or the composition of any of paragraphs 55-56; or the cell of paragraph 58, and a pharmaceutically acceptable carrier.

60. A solid support comprising the antibody, antibody reagent, antigen-binding fragment thereof, or CAR of any of paragraphs 39-54.

61. The solid support of paragraph 60, wherein the antibody, antibody reagent or antigen-binding fragment thereof is detectably labeled.

62. The solid support of any of paragraphs 60-61, wherein the solid support comprises a particle, a bead, a polymer, or a substrate.

63. A kit for the detection of PD1 polypeptide in a sample, the kit comprising at least a first antibody, antibody reagent, antigen-binding fragment thereof, or CAR of any of paragraphs 39-54 immobilized on a solid support and comprising a detectable label.

64. A molecular complex comprising at least one antibody, antibody reagent, antigen-binding fragment thereof, or CAR of any of paragraphs 39-54 bound to an PD1 polypeptide.

65. A method of treating cancer in a subject in need thereof, the method comprising administering the antibody, antibody reagent, antigen-binding fragment thereof, or CAR of any of paragraphs 39-54; or the composition of any of paragraphs 55-56; or the cell of paragraph 58, to the subject. 66. The method of paragraph 65, wherein the cancer is selected from the group consisting of:
Non-small cell lung cancer; melanoma; metastatic melanoma; renal cell carcinoma; squamous cell carcinoma of the head and neck; Hodgkin lymphoma; classical Hodgkin lymphoma; and urothelial carcinoma.

67. The antibody, antibody reagent, antigen-binding fragment thereof, or CAR of any of paragraphs 39-54; or the composition of any of paragraphs 55-56; or the cell of paragraph 58, for use in a method of treating cancer in a subject in need thereof.

68. The composition of paragraph 67, wherein the cancer is selected from the group consisting of:
Non-small cell lung cancer; melanoma; metastatic melanoma; renal cell carcinoma; squamous cell carcinoma of the head and neck; Hodgkin lymphoma; classical Hodgkin lymphoma; and urothelial carcinoma.

### EXAMPLES

V(D)J recombination generates mature B cells that express huge repertoires of primary antibodies as diverse immunoglobulin heavy (IgH) and light chains (IgL) of their B cell antigen receptors (BCRs). Cognate antigen binding to BCR variable region domains activates B cells into the germinal center (GC) reaction in which somatic hypermutation (SHM) modifies primary variable region-encoding sequences, with subsequent selection for mutations that improve antigen-binding affinity, ultimately leading to antibody affinity maturation. Based on these principles, a humanized mouse model approach was developed to diversify an anti-PD1 therapeutic antibody, which allows isolation of variants with novel properties. In this approach, component Ig gene segments of the anti-PD1 antibody underwent de novo V(D)J recombination to diversify the anti-PD1 antibody in the primary antibody repertoire in the mouse models. Immunization of these mouse models further modifies the anti-PD1 antibodies through SHM. Known anti-PD1 antibodies block interaction of PD1 with its ligands to alleviate PD1-mediated T cell suppression, thereby boosting anti-tumor T cell responses. By diversifying one such anti-PD1 antibody, many new anti-PD1 antibodies were derived, including anti-PD1 antibodies with the opposite activity of enhancing PD1/ligand interaction. Such antibodies may suppress deleterious T cell activities in autoimmune diseases.

### Example 1

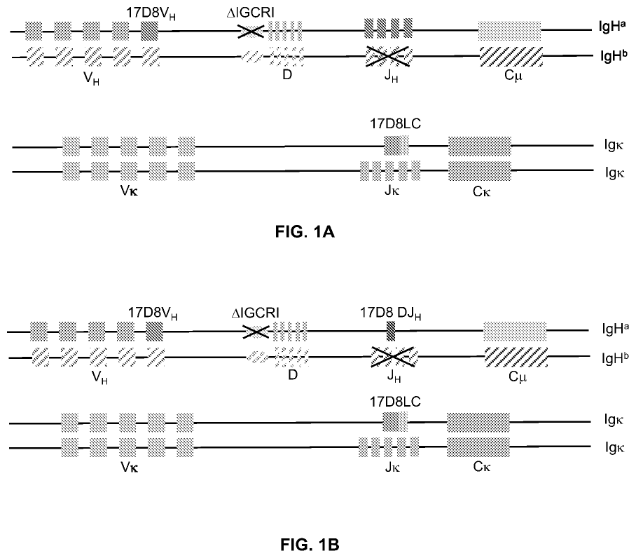

Referring generally to FIGS. 1A-1B, diagrams of the IgH and Igk loci of the mouse models that were used to diversify anti-PD1 antibody, 17D8, are illustrated, according to some implementations of the present disclosure. The disclosed anti-PD1 antibodies were isolated with a method developed to diversify existing therapeutic antibodies. The immunoglobulin heavy chain variable region of therapeutic antibody was separated into V_{H} and DJ_{H} components, and the gene segments were incorporated into the mouse IgH locus (FIG. 1A). During B-cell maturation, V_{H} and DJ_{H} segments is rejoined via V(D)J recombination; during this process, the loss or gain of random nucleotides at the recombination junction diversifies an important part of the antigen-binding site, CDR H3. As a result, the mouse produces a library of antibodies with subtle differences in antigen-binding properties. Immunization of the mice with target antigen selects out best binders for the antigen, and induces further affinity maturation of the antibodies.

The method disclosed above was tested by diversifying an existing anti-PD1 antibody, 17D8 (U.S. Pat. No. 8,008,449 B2), which was developed by Bristol-Myers Squibb and Ono Pharmaceutical for the treatment of melanoma. The antibody is closely related to Nivolumab, the anti-PD1 antibody that is currently used in the clinic.

As shown in FIG. 1A, in mouse model 1, the 17D8VH replaces mouse VH81X segment. Deletion of IGCRI increases the rearrangement of 17D8V_{H}. The other IgH allele was inactivated by deletion of the J_{H} region. A pre-rearranged 17D8LC variable region exon was integrated into one of Jk alleles. To diversify the 17D8 antibody, its heavy chain (HC) variable region was divided into 17D8VH and DJH segments. The 17D8VH segment was used to replace the mouse VH81X segment in mice (mouse model 1, FIG. 1). Relative to other VH segments, VH81X is used preferentially for V(D)J recombination; deletion of a regulatory element between the VH and D segments, IGCRI, further accentuates the bias (FIG. 1A). The 17D8VH segment, in place of VH81X, also dominates IgH rearrangement in the mouse model. More important, the 17D8VH segment recombines with random mouse D and JH segments, thereby creating a diverse range of CDR H3s.

As shown in FIG. 1B, mouse model 2 is the same as mouse model 1 (FIG. 1A), except for the integration of the DJ_{H} segment of 17D8 HC. In another version of the mouse model (mouse model 2, FIG. 1B), the 17D8 DJH segment was also incorporated into the mouse JH locus. In this configuration, the 17D8VH segment recombines with 17D8 DJH segment to diversify the junction between VH and DJH segments. Mouse model 2 generates antibodies that are more closely related to the original 17D8 antibody than mouse model 1.

To complement 17D8HC expression, a pre-rearranged 17D8 light chain (LC) was integrated into the mouse Jκ locus in both mouse models (FIGS. 1A and 1B). These genetic modifications were carried out in mouse embryonic stem (ES) cells, and the ES clones were injected into Rag2 deficient blastocysts to generate chimeric mice. Because Rag2 is essential for V(D)J recombination, all the B- and T-cells in the chimeric mice are derived from the injected ES cells; this method obviates the expensive and time-consuming process of mice breeding.

The mouse model 1 and the mouse model 2 were immunized; and multiple novel anti-PD1 antibodies were isolated. Referring now to FIG. 2, the effect of various antibodies on the interaction between PD1 and PD-L1 is illustrated in five graphs, according to some implementations of the present disclosure. In this example, a PD1 expressing cell line was incubated with PD-L1 or PD-L1 plus various antibodies. The binding activity of PD-L1 was displayed as histograms, and the binding profiles for PD-L1 (blue) and PD-L1 plus antibody (red) were overlaid. The x-axis represents the level of PD-L1 associated with PD1 on cell surface; the y-axis represents the relative cell number, and highest peak was set as 100%. 319-9-8 is an antibody that retained the CDR H3 of 17D8, and this antibody inhibits the binding of PD-L1 to PD1, as the original 17D8 antibody. 319-9-27, 319-9-34 and 397-27-25 are antibodies that have distinct CDR H3s from 17D8, and these antibodies enhance the interaction between PD-L1 and PD1.

The 17D8 antibody, like the antibodies in clinical application (Nivolumab and Perbrolizomab), blocks the interaction between PD-1 and its ligand PD-L1. By contrast, some of the disclosed anti-PD1 antibodies (397-27-25, 319-9-27 and 319-9-34) enhance the binding of PD-1 to PD-L1 (FIG. 2). Such type of antibodies can potentially be used to suppress deleterious T cell activities, for example, in autoimmune diseases. Furthermore, this result shows that the disclosed antibody diversification method can derive novel antibodies with distinct functions from the prototype.

### Example 2

The immune system is capable of producing enormous varieties of antibodies to counter pathogenic infections. In this example, a method was devised to harness the power of the immune system to produce potentially therapeutic antibodies. This example was used to generate antibodies against the human PD1 molecule, a negative regulator of T cell activity. Available anti-PD1 therapeutic antibodies inhibit the function of PD1, thereby boosting T cell activity against tumor cells. Through this example, one such anti-PD1 antibody was diversified in the mouse models. From this example, multiple new anti-PD1 antibodies were obtained, some of which can stimulate, rather than inhibit, PD1 function in vitro. Such PD1 stimulatory antibodies may be used to suppress pathogenic T cell activities in autoimmune diseases.

An in vivo method for antibody diversification and optimization was utilized in this example, which exploits antibody diversification mechanisms during B cell development and activation in mice. At the progenitor B cell stage, V(D)J recombination joins Ig V_{H}, D and J_{H} gene segments into complete exons that encode IgH variable regions of antibodies and similarly joins the V_{L} and J_{L} segments that encode the variable regions of IgL chains of antibodies. A major portion of antibody diversity comes from mechanisms that diversify the junctions of V, D, and J segments during V(D)J recombination. Thus, as the portions of the V_{H}-D and D-J_{H} junctions of antibody IgH chain variable regions or the V_{L}-J_{L} junctions of IgL chain are part of the antigen-contact complementarity-determining region 3 (CDR3) of IgH and IgL chains, junctional diversification generates enormous varieties of primary antigen-binding B cell receptors (BCRs) for any given combination of germline encoded V, D and J segments. Unique BCRs are displayed on the surface of each clonally generated primary B cell, which then migrate to peripheral lymphoid tissues. There, antigen binding to a cognate B cell receptor stimulates the corresponding B cells, which ultimately can participate in germinal center (GC) reactions. The Ig variable regions of GC B cells accumulate SHMs that can further diversify IgH and IgL CDR3 sequences, as well as the two other antigen-contact CDR1 and CDR2 encoded in germline V_{H} and V_{L} gene segments. Some SHMs increase antigen-binding affinity of the B cell receptor, and the germinal center microenvironment selects for B cells with increased antigen-binding affinity. Repeated cycles of mutation and selection can lead to antibody affinity maturation.

For this example, mice that predominantly assemble the IgH V(D)J exon of an existing therapeutic antibody were generated by V(D)J recombination during B cells development, creating vast repertoires of primary B cells expressing different variations of the antibody due to junctional diversification of the IgH variable region CDR3. Then these mice were immunized with the cognate antigen for the therapeutic antibody to further diversify the primary antibody sequences by SHM and affinity maturation in the GC. Relative to in vitro antibody development platforms, such as phage display or yeast display, this example in vivo approach yields some antibodies that are more suitable for clinical applications. For example, B cell developmental checkpoints can eliminate poly-reactive antibodies. Moreover, B cell survival depends on functional B cell receptors; this requirement selects in vivo for antibodies with stable and normal conformations. For clinical application, antibodies are usually produced in mammalian cells. When antibodies are expressed in mouse B cells, functional properties selected in mice are more likely to be preserved than those for in vitro evolved antibodies from bacteria or yeast.

The ability of the disclosed in vivo antibody diversification approach was tested to generate new versions of anti-PD1 antibody from an existing prototype. PD1 is a surface receptor on activated T cells. Interaction of PD1 with its ligands represses T cell activity. By blocking the association between PD1 and its ligands, antibodies against PD1 or its ligand can neutralize the PD1-dependent negative regulatory pathway, thereby boosting T cell activity. Based on this function, anti-PD1 antibodies have proved effective in cancer immunotherapy. Further diversification of existing anti-PD1 antibodies may potentiate their efficacy for current uses or alter their activity for new applications. Based on the in vivo diversification approach, a set of new anti-PD1 antibodies was generated, several of which actually enhance, rather than block, PD1 interaction with its ligand.

In this example, an anti-PD1 antibody 17D8 was chosen to produce variant antibodies with new properties. Since the IgH or IgL variable region sequences of the 17D8 antibody are close to the germline versions of the component Ig gene segments, the antigen-recognition specificity and affinity of this antibody may be further modified by SHM. To rapidly generate a mouse model system to test the 17D8 in vivo antibody diversification approach, genetic modifications described herein were introduced into mouse embryonic stem (ES) cells and then injected the engineered ES cells into Recombination Activating Gene-2 (RAG-2) deficient blastocysts to generate chimeric mice, in which all mature lymphocytes develop from the injected ES cells with the entire set of in vitro-introduced genetic modifications. With this RAG-2 deficient blastocyst complementation approach, the chimeric mice can be used directly for immunization experiments, thereby saving the expense and time associated with conventional germline breeding.

To diversify the 17D8 antibody via junctional diversification during progenitor B cell development, its IgH variable exon was separated into its component V_{H} and recombined DJ_{H} segments, derived from human V_{H}3-33, D1-1 and J_{H}4 segments, respectively. The 17D8 V_{H} Segment was then substituted for the most proximal mouse V_{H}81X gene segment in ES cells, in which the IGCRI regulatory region was deleted to render the integrated human 17D8 V_{H} segment by far the most utilized V_{H} in developing B cells of RDBC chimeras derived from these ES cells (FIG. 1A). To further enrich the representation of the 17D8 V_{H} and DJ_{H} segments in primary antibody repertoire, the J_{H} region of the other IgH allele was also deleted in the targeted ES cells (FIG. 1A). To reconstitute the complete 17D8 HC variable region during V(D)J recombination in developing RDBC progenitor B cells, the mouse DQ52-J_{H} region in the modified ES cells was replaced with the recombined 17D8 DJ_{H} segment. A recombination signal sequence (RSS), upstream of the 17D8 DJ_{H} segment, enables the joining of the 17D8 DJ_{H} segment with upstream V_{H} segments, including the 17D8 V_{H} Segment in developing mouse progenitor B cells (FIG. 1A). Because the RSS of the assembled 17D8 DJ_{H} segment was derived from a mouse D segment, the 12/23 rule of V(D)J recombination restricts the recombination of the 17D8 DJ_{H} segment RSS to upstream V_{H} RSS, but is not the incompatible D RSS for joining.

After V(D)J recombination, the V_{H}-DJ_{H} junction can gain or lose random nucleotides; such junctional diversification can create tremendous variations in CDR H3. Thus, V(D)J recombination of 17D8 V_{H} with DJ_{H} in vivo does not simply regenerate the original 17D8 antibody heavy chain (HC); the process will produce a large library of related HCs that differ in CDR H3 length and/or sequence. Some of the new CDR H3s may recognize PD1 with equal or higher affinity than the original CDR H3 in 17D8 or even target different epitopes. Mice generated with this diversification strategy is referred to as PD1 Diversification Mouse Model 1.

In a variation of this strategy, referred to as PD1 Diversification Mouse Model 2, the 17D8 DJ_{H} segment was not incorporated into the mouse IgH locus (FIG. 1B), allowing the 17D8 V_{H} segment to undergo V(D)J recombination with mouse D and J_{H} segments, with junctional diversification occurring at both V_{H}-D and D-J_{H} joints. As a result, the CDR H3 diversity in this model should be even higher than that in PD1 Diversification Mouse Model 1 with the 17D8 pre-rearranged DJ_{H} Segment. CDR H3 often retains minimal remnants of the D segment, and mouse J_{H}s are homologous to human counterparts. Furthermore, due to junctional diversification, human antibodies also contain highly heterogenous CDR H3s. So, mouse CDR H3s may not pose a major problem for human applications.

To complement 17D8 HC expression, a pre-rearranged (V_{κκ}) variable region (V) exon for the 17D8 light chain (LC) was integrated into the mouse Jκ locus (FIGS. 1A and 1B) in ES cells, which already harbored the modified IgH locus for PD1 Diversification Mouse Model 1 or 2. The resultant ES clones, now engineered to express both diversified 17D8 HCs and a CDR L3-prefixed 17D8 LC were used to generate chimeric mice by RDBC. CDR L3 is much less diverse than CDR H3, because CDR L3 lacks a D segment and involves only V_{L}-J_{L} junctions.

In addition, in some implementations, CDR H3s are diversified via terminal deoxynucleotide transferase (TdT)-mediated N region additions to recombination junctions; while CDR L3s in mouse IgL variable regions are not diversified by N-nucleotide addition, due to the absence of TdT expression in mouse precursor-B cells that undergo V_{L} to J_{L} joining. For these reasons, we did not aim to diversify the 17D8 CDR L3 through V(D)J recombination in this set of mouse models. In addition, one advantage of expressing a pre-rearranged human V exon for the 17D8 LC is that, due to negative feedback regulation from the expressed rearranged knock-in IgL chain, most naïve B cells in the mouse model should express the 17D8 LC. Thus, in conjunction with the dominance of the diverse 17D8-derived HCs, the majority of B cells in these two mouse models should express 17D8-related antibodies with a diverse range of CDR H3s.

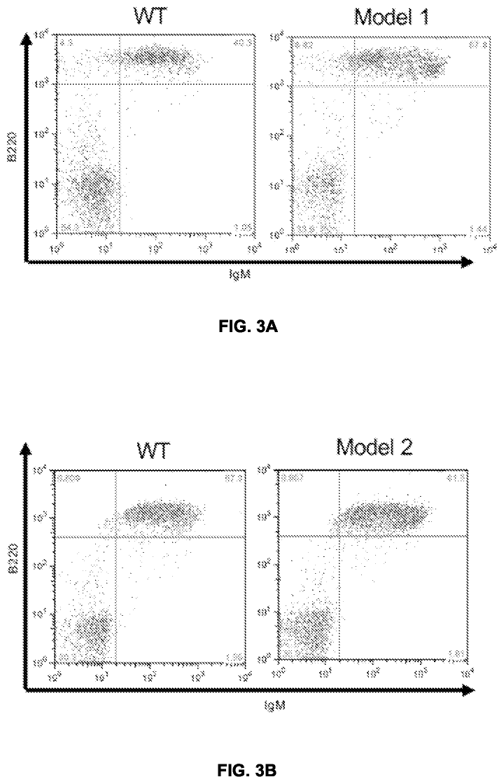

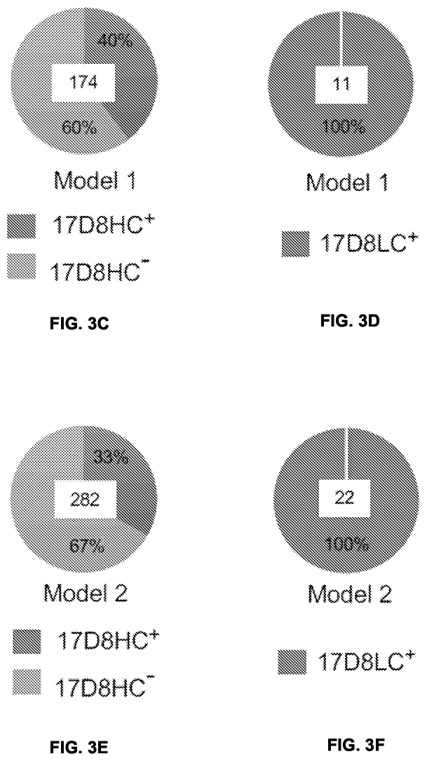

Referring to FIGS. 3A-3F, analyses of the mouse models to diversify an anti-PD1 antibody, 17D8, were shown. FIG. 3A depicts FACS analysis of B cells in blood from wild-type 129/Sv mouse and mouse model 1. The surface markers detected by the FACS staining are indicated next to the axis. FIG. 3B depicts FACS analysis of B cells in blood from wild-type 129/Sv mouse and mouse model 2. The surface markers detected by the FACS staining are indicated next to the axis. FIG. 3C depicts Hybridomas were generated from splenic B cells of mouse model 1. IgH rearrangements involving the 17D8 V_{H} and DJ_{H} segments in each hybridoma clone were detected with PCR. In total, 174 hybridoma clones, the number at the center of pie chart, were analyzed, and 40% of the clones were positive for 17D8 V_{H}(D)J_{H} recombination products (17D8HC⁺). FIG. 3D depicts the fraction of Igk transcripts corresponding to 17D8LC in mouse model 1. 5′RACE was performed from Ck, using RNA from splenocytes of mouse model 1. 11 PCR products, the number shown at the center of the pie chart, were sequenced, and all of them corresponded to the 17D8LC. FIG. 3E depicts the percentage of IgH rearrangements involving the 17D8 V_{H}, mouse D and J_{H} segments in mouse model 2. IgH rearrangements involving the 17D8 V_{H}, mouse D and J_{H} segments in each hybridoma clone were detected with PCR. In total, 282 hybridoma clones, the number at the center of pie chart, were analyzed, and 33% of the clones contained rearrangements involving the 17D8 VH. FIG. 3F depicts the fraction of Igk transcripts corresponding to 17D8LC in mouse model 2. 5′RACE was performed from Ck, using RNA from splenocytes of mouse model 1. 22 PCR products, the number shown at the center of the pie chart, were sequenced, and all of them corresponded to the 17D8LC.

Based on FACS analysis of blood lymphocytes, the RDBC chimeric mice for both mouse models had comparable B cell populations as wild-type 129/Sv mice (FIGS. 3A and 3B). As described herein, a large fraction of B cells should express HCs containing the 17D8 V_{H} and DJ_{H} segments in PD1 Diversification Mouse Model 1, or 17D8 V_{H} segment in association with mouse D and J_{H} segments in PD1 Diversification Mouse Model 2. To confirm this expectation, hybridomas from splenic B cells of these mice were generated. Indeed, 40% of B cell hybridomas from PD1 Diversification Mouse Model 1 contained 17D8 V_{H}-DJ_{H} recombination products (FIG. 3C). Since the JH region has been deleted from the other IgH allele, all the 17D8 HC rearrangements are productive and support B cell survival. Similarly, in PD1 Diversification Mouse Model 2, the 17D8 V_{H} segment recombined with mouse Ds and J_{H}s in approximately 33% of B cell hybridomas (FIG. 3C). Due to feedback regulation from the knock-in 17D8 variable exon-encoded IgL chain, rearrangement of the endogenous Igκ locus was inhibited in developing B cells in the RDBC chimeric mice, and all the Igκ transcripts from the splenic B cells of both mouse models corresponded to the 17D8 LC (FIGS. 3D and 3F).

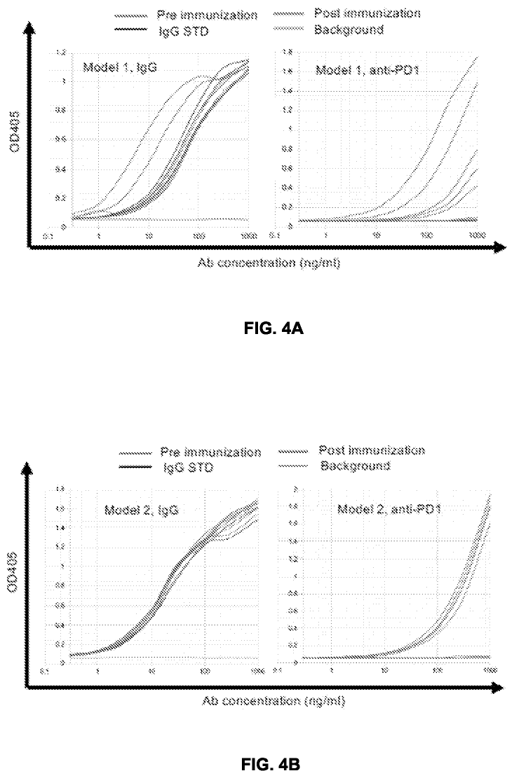

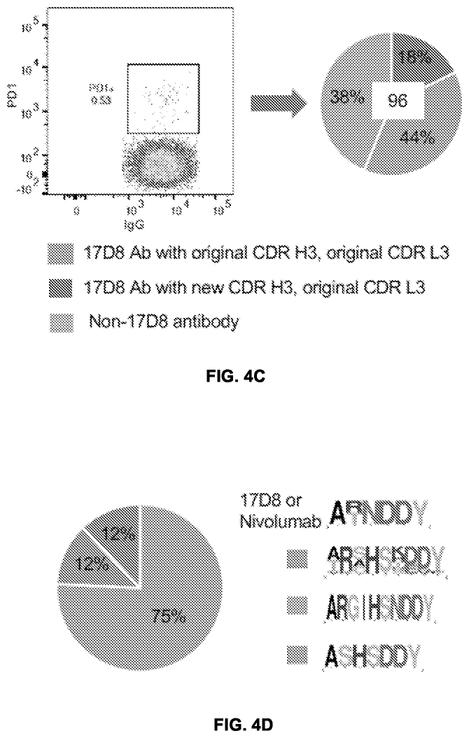

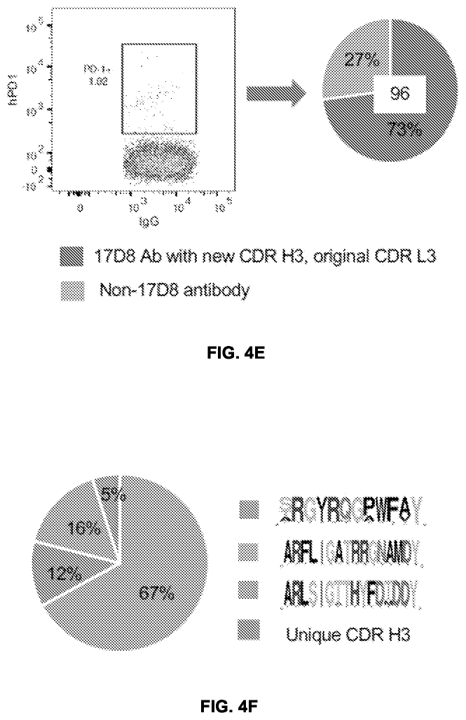

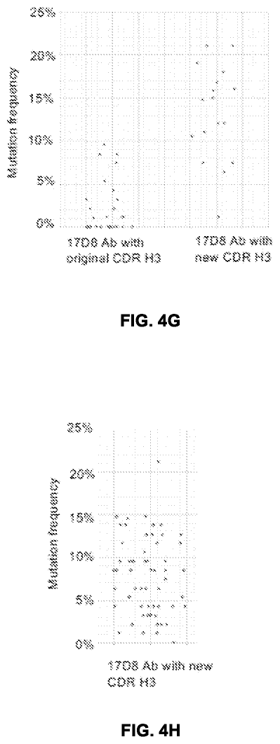

In both PD1 Diversification Mouse Models, V(D)J recombination diversifies the CDR H3 of the 17D8 antibody. At least a subset of the new CDR H3s may be able to interact with PD1, but with different affinities or targeting distinct epitopes from the original CDR H3 in the 17D8 antibody. Immunization of these mouse models with PD1 can activate naïve B cells that express new anti-PD1 antibodies. Some of the activated B cells will undergo SHM that might further influence their PD1 binding affinity. For immunization of these mouse models, a fusion protein that consists of the extracellular domain of the human PD1 and Glutathione-S-transferase (GST) was used. The GST portion facilitates affinity purification of the protein and may provide extra epitopes for helper T cells, which are critical for affinity maturation during GC reaction. In this regard, thymic tolerance mechanisms should purge T cells that recognize mouse PD1 epitopes, as well as homologous epitopes in human PD1. The exogenous GST can compensate for the potential paucity of helper T cells that recognize antigenic peptides from the human PD1. Immunization with PD1-GST can induce antibodies against both the PD1 and GST portion of the fusion protein. To specifically detect anti-PD1 antibodies in ELISA, PD1 fused to human IgG4 fragment crystallizable (Fc) region was used; this fusion protein lacks the GST portion of the immunogen and should only interact with anti-PD1 antibodies from the immunized mice. Serum from unimmunized mice did not contain detectable PD1 binding activity in ELISA (FIGS. 4A and 4B). Two rounds of immunization with PD1-GST induced robust anti-PD1-binding IgG in plasma from both mouse models (FIGS. 4A and 4B).

FIG. 4A depicts ELISA detection of anti-PD1 IgG in plasma after immunization of mouse model 1. The plot on the left shows IgG concentrations in mouse plasma, collected two weeks after the second immunization; the plot on the right shows anti-PD1 IgG levels in the same set of plasma samples as in the IgG plot. The x-axis represents plasma concentration; the highest plasma concentrations of different samples were adjusted to have about 1 μg/ml IgG, as defined by the IgG standard (IgG STD, black curve). The blue and red curves represent titration of from pre- or post-immune plasmas, respectively. Each curve is the result of analysis of plasma sample from one mouse. The background (green curve) was defined by buffer. FIG. 4B depicts ELISA detection of anti-PD1 IgG in plasma after immunization of mouse model 2. The plots are results of the same type of analysis as shown in FIG. 4A.

FIG. 4C depicts sorting of PD1-binding B cells from immunized mouse model 1 and cloning of 17D8 antibody or its variants. Splenic B cells from immunized mouse model 1 were first enriched for memory B cells with MACS purification kit. B220⁺IgG⁺PD1⁺ B cells, shown in the PD1⁺ gate with frequency, were sorted as single cells into 96 well plate. The HCs and LCs of the single cells were amplified with primers specific to the 17D8 V_{H}/Cγ or 17D8 V_{L}/Ck. Antibodies with both 17D8 VH and V_{L} were defined as 17D8 antibodies (17D8 Ab), which were further separated into two categories, based on their CDR H3s. The pie chart shows the distribution of the three types of antibodies from the sorted single B cells; in total, 96 cells, the number at the center of the pie chart, were analyzed.

For DNA sequence analyses of anti-PD1 antibodies, fluorophore-conjugated PD1-Fc protein was used as a probe to sort IgG+PD1-specific splenic B cells from the immunized Mouse Model 1 (FIG. 4C) and cloned paired HC and LC from individual B cells with single-cell RT-PCR. Primers specific to the 17D8 HCs and LCs were used for nucleotide sequence analysis of IgG+PD1-specific splenic B cells isolated from both mouse models. From the spleen of one immunized PD1 Diversification Mouse Model 1, 62% of 96 sorted single B cells expressed both the 17D8 HC and LC (blue and orange portion in the pie chart, FIG. 4C); the other 38% sorted B cells (grey portion in the pie chart, FIG. 4C) did not yield any PCR products with the 17D8 HC-specific primer, presumably because these B cells expressed mouse IgH variable regions. Among the 62% of sorted B cells, which expressed both 17D8 HCs and LCs, 44% of the HCs had the same CDR H3 as the original 17D8 HC (orange portion in the pie chart, FIG. 4C); in these cases, the joining of 17D8 VH segment to the DJ_{H} segment restored the original CDR H3 of the 17D8 HC, which was then selected into the pool of IgG+ activated B cells by PD1 immunization.

Recovery of the original 17D8 HC, subsequent to its variable region exon assembly by V(D)J recombination, confirmed that the immunization and antibody isolation procedures were highly specific for anti-PD1 antibodies. The other 18% of the HCs had CDR H3s that diverged from the 17D8 CDR H3 both in sequence and length, due to diversification at the VH-DJH junction (the blue portion of the pie chart, FIG. 4C).

Based on length and sequence, the new PD1 specific CDR H3s can be divided into three groups, which may have derived from clonal expansion of three main precursors (FIG. 4D). FIG. 4D depicts profiles of new CDR H3s in 17D8 antibody variants from mouse model 1. The logo plots show the CDR H3s, from top to bottom, in the original 17D8 antibody and related Nivolumab and three new types of novel anti-PD1 antibodies derived from 17D8. The pie chart shows the distribution of the three new types of CDR H3s. Most of the new CDR H3s shared amino acid sequences at the N-terminal (“AR”) and C-terminal regions (“DDY”) with the original 17D8 CDR H3, because these residues were encoded by the VH and the assembled DJH segments, respectively. The Asn (“N”) amino acid residue at the upstream end of the DJH segment of the 17D8 CDR H3 was prone to be altered during VH-DJH joining, as shown in the new CDR H3s (FIG. 4D). All the LCs paired with 17D8 HCs corresponded to the pre-rearranged 17D8 LC (FIG. 4C), which was expressed in most naïve B cells (FIGS. 3D and 3F).

The same DNA sequence analysis of the anti-PD1 antibodies was performed by sorting IgG+PD1 specific B cells from immunized Mouse Model 2 (FIG. 4E). FIG. 4E depicts sorting of PD1-binding B cells from immunized mouse model 2 and cloning of 17D8 antibody variants. This experiment was performed in the same manner as that shown in FIG. 4C. From the spleen of one immunized Mouse Model 2, 73% of 96 sorted single B cells expressed both the 17D8 HC and LC (the blue portion of the pie chart, FIG. 4E). The remaining 27% of sorted B cells did not yield positive signals for 17D8 HC in single-cell PCR and presumably expressed mouse IgH variable regions (the grey portion in the pie chart, FIG. 4E).

In the PD1 Diversification Mouse Model 2, the 17D8 VH recombined with mouse Ds and JHs, and all HCs had distinct CDR H3s from the 17D8 antibody. Based on CDR H3 sequence, most of the antibodies belonged to three groups (FIG. 4F). FIG. 4E depicts profiles of new CDR H3s in 17D8 antibody variants from mouse model 2. The panel is organized in the same way as in FIG. 4D. Each group likely arose from clonal expansion of a founder B cell that expressed a primary antibody with the primordial CDR H3. Again, all the LCs paired with 17D8 HCs corresponded to the pre-rearranged 17D8 LC (FIG. 4E).

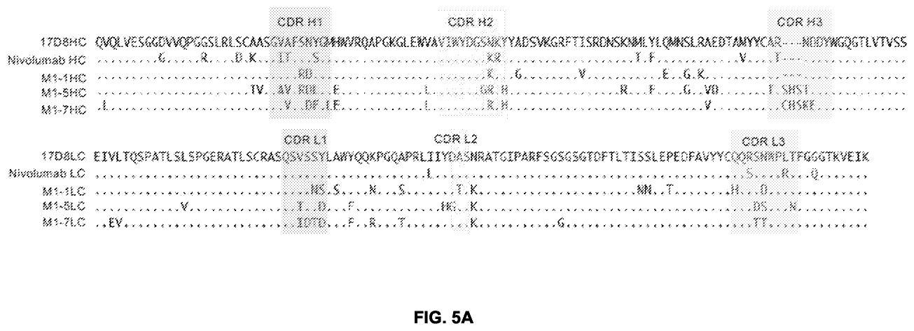

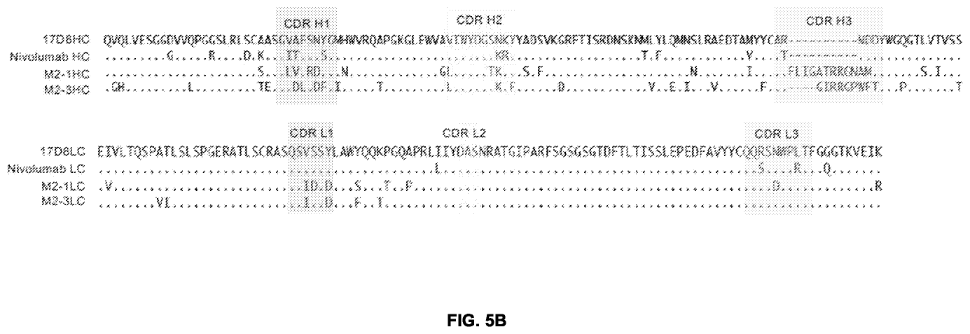

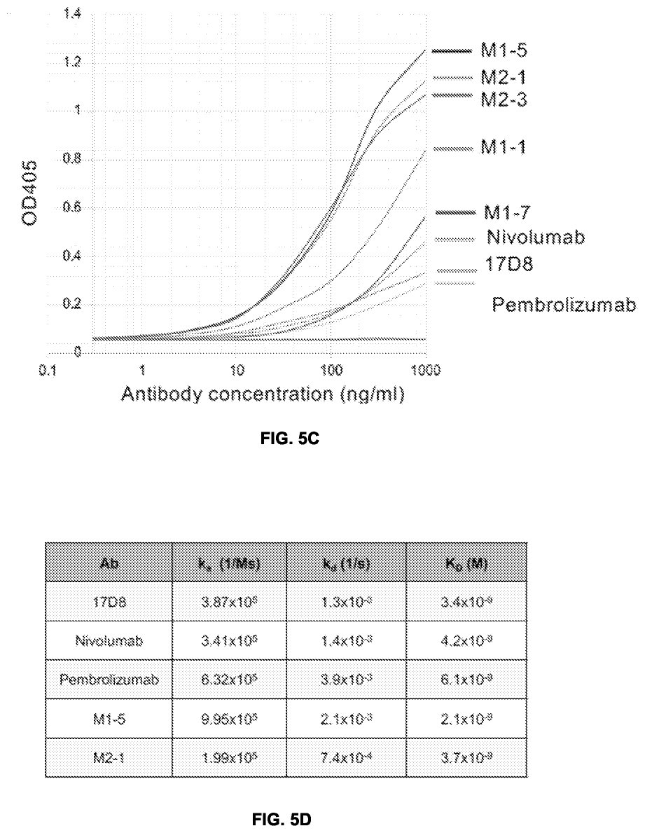

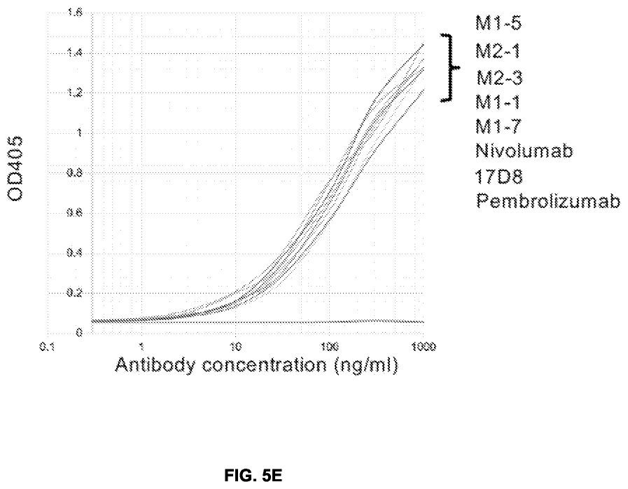

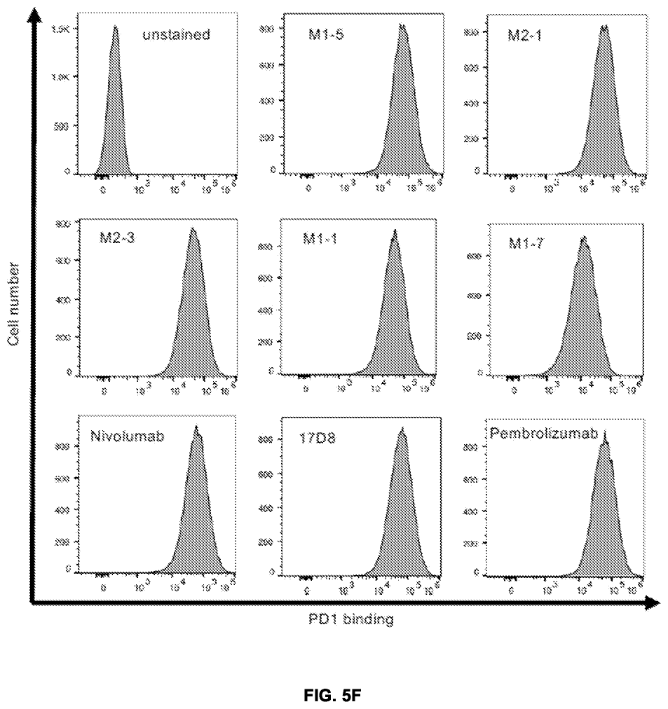

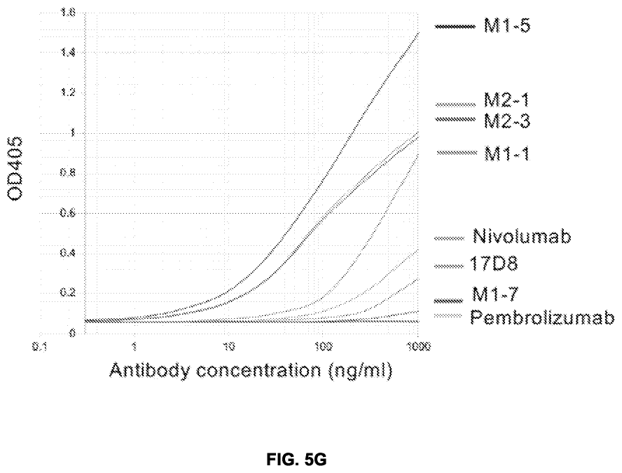

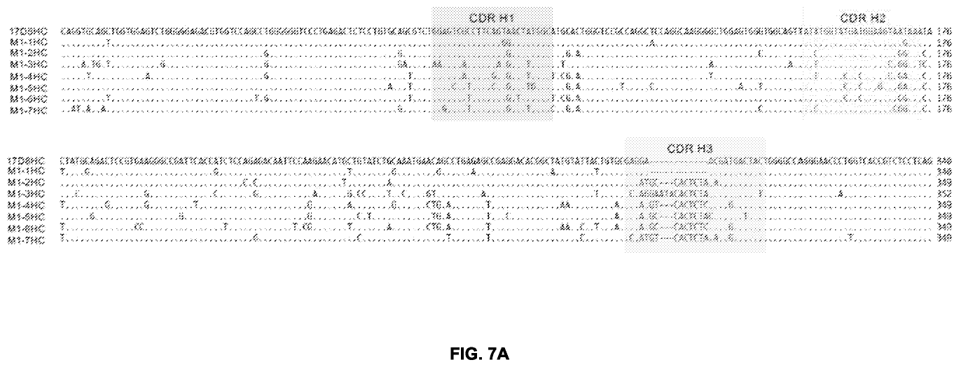

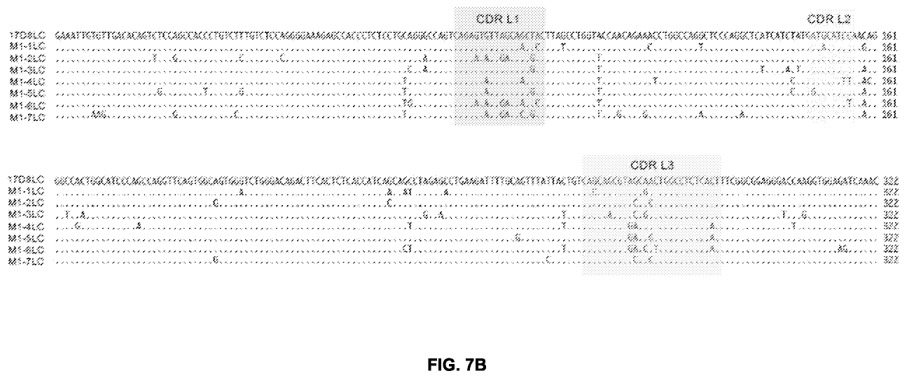

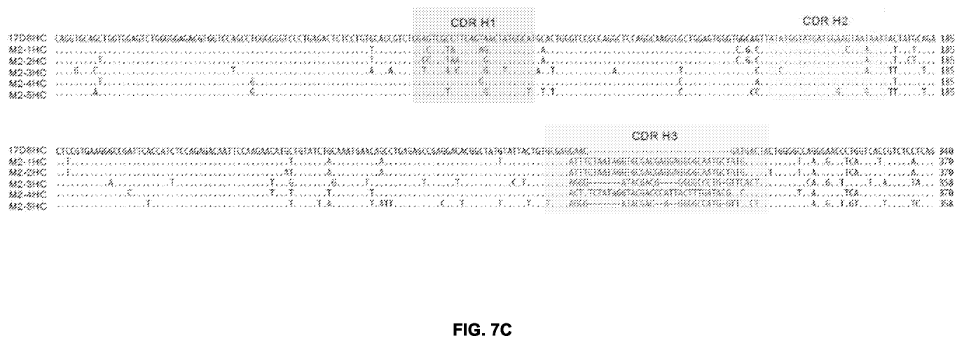

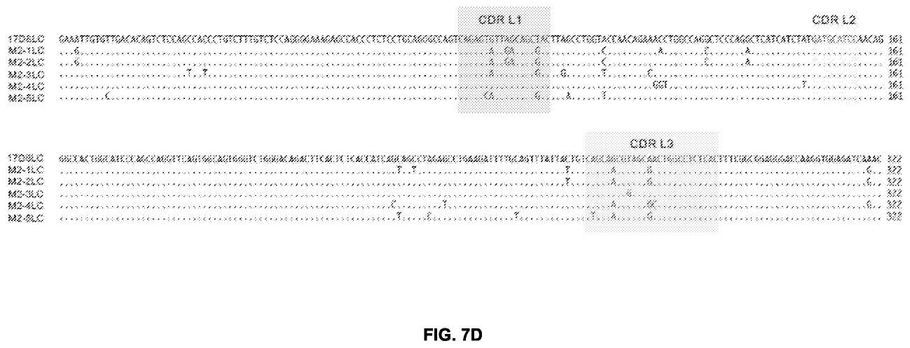

In addition to CDR H3 diversification in PD-1 Diversification Mouse Model 1, SHM occurred throughout the reassembled 17D8 HC and the knock-in 17D8 LC variable region exons (FIGS. 7A and 7B, FIG. 5A and FIG. 8A). FIG. 5A depicts sequence comparisons of three new anti-PD1 antibodies from Mouse Model 1 with the 17D8 antibody and Nivolumab. The top and bottom parts of this section show the alignments of the amino acid sequences of IgH (HC) and IgL (LC) variable regions, respectively. In the alignment, only amino acid residues that differ from the 17D8 sequence are shown; “.” Indicates identity; “-” represents gap in alignment. The CDRs are shaded with colors. FIG. 7A depicts alignment of M1-1 to M1-7HCs with the 17D8HC. The sequence of the 17D8HC serves as the template for alignment; only SHMs in new anti-PD1 antibodies are shown below, “.” Indicates identity, “-” indicates gap in alignment. The CDRs are shaded with color. FIGS. 5B-5D are organized in the same way as FIG. 5A. FIG. 7B depicts alignment of M1-1 to M1-7LCs with the 17D8LC.

Overall, the PD1 Diversification Mouse Model 1 HCs with new CDR H3s had higher frequencies of amino acid changes, due to SHM, than the original 17D8 HC (FIG. 4G). FIG. 4G depicts frequencies of amino acid changes in the 17D8 antibody heavy chains with the original CDR H3 and new CDR H3s from mouse model 1. Each dot represents one antibody heavy chain. The difference may be attributable to the initial PD1 binding strength of their respective primary antibodies. Since the 17D8 antibody already binds strongly to PD1, SHM may not increase antigen-binding affinity substantially to confer selective advantage during the GC reaction. In contrast, primary antibodies with new CDR H3s may bind PD1 less stably than the 17D8 antibody, consequently allowing strong selection for mutations that increased PD-1 binding affinity. FIG. 4H depicts frequencies of amino acid changes in the heavy chains of 17D8 antibodies with new CDR H3s in mouse model 2.

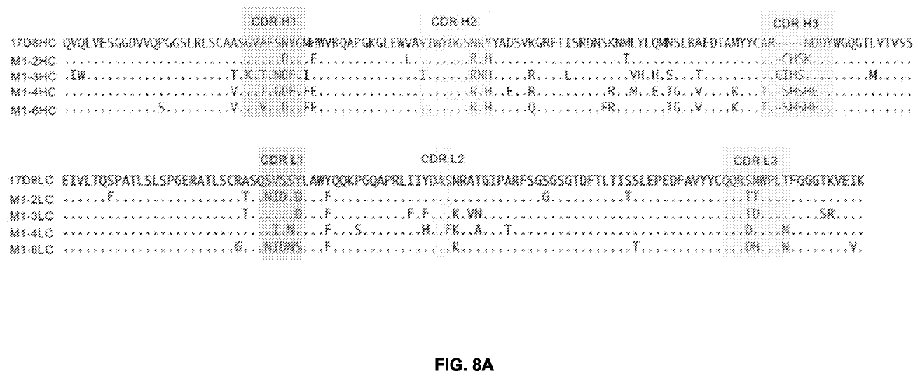

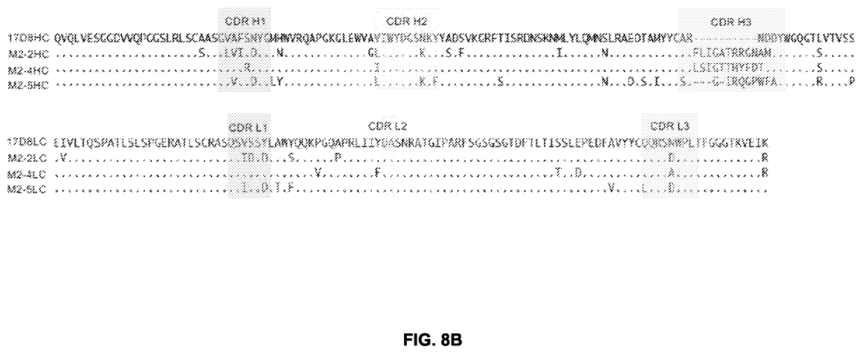

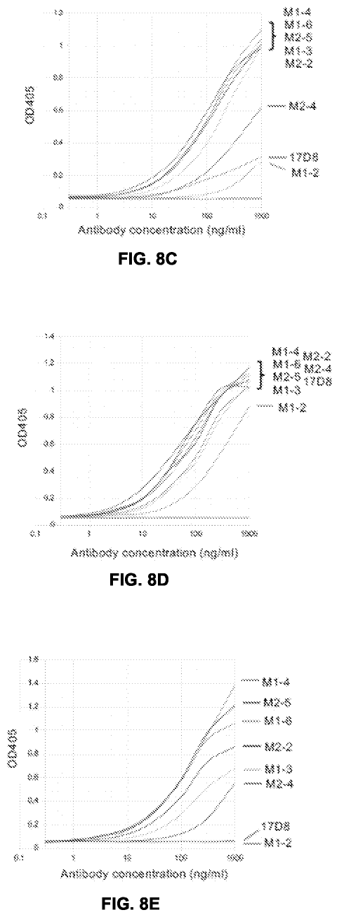

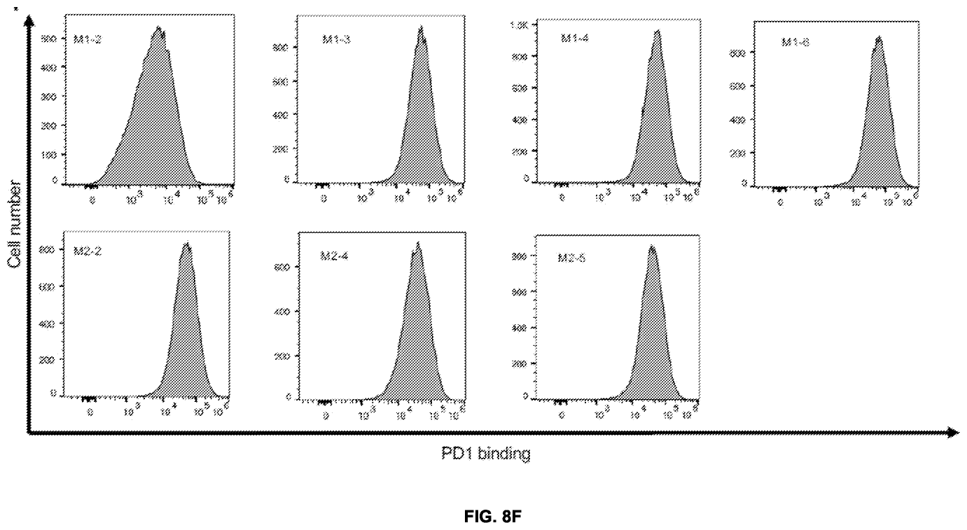

As a sign of antigenic selection, some of the amino acid changes (FIG. 5A and FIG. 8A), as well as the underlying SHMs (FIG. 7A), were recurrent among different antibodies, and acquired mutations appeared more concentrated in CDRs (e.g. the N to D mutation in CDR H1 and N to K or R mutation in CDR H2, FIG. 5A and FIG. 7A). Like antibodies with new CDR H3s from Model 1, most of the antibodies from Model 2 contained substantial levels of SHMs (FIGS. 7C and 7D) and corresponding amino acid changes (FIG. 4H, FIG. 5B and FIG. 8B). As hypothesized above, the primary antibodies for these new anti-PD1 antibodies probably interacted with PD-1 weakly, leaving ample room for affinity maturation by SHM.

To test PD1 binding activity of the new antibodies, some of the cloned HC and LC pairs were expressed as recombinant antibodies. Twelve antibodies representing different CDR H3 groups were chosen from the two mouse models (FIGS. 5A and 5B, FIGS. 7A-7D, FIGS. 8A and 8B). The 7 antibodies isolated from PD1 Diversification Mouse Model 1 were referred to as M1-1 to M1-7; and the 5 antibodies isolated from PD1 Diversification Mouse Model 2 were referred to as M2-1 to M2-5 (FIGS. 5A-5B, FIGS. 7A-7D, FIGS. 8A-8B). For comparison, the 17D8 antibody, Nivolumab and Pembrolizumab were produced; the latter two antibodies are used in cancer immunotherapy.

FIG. 5B depicts sequence comparisons of two new anti-PD1 antibodies from Mouse Model 2 with the 17D8 antibody and Nivolumab. The top and bottom parts of FIG. 5B show the alignments of the amino acid sequences of IgH (HC) and IgL (LC) variable regions, respectively. FIGS. 7A-7D depict DNA Sequence alignments of the Ig variable regions of new anti-PD1 antibodies with the 17D8HC.

ELISA was used to compare the PD1 binding activities of the new and previous anti-PD1 antibodies. The ELISA coating antigen was a recombinant protein of the extracellular domain of PD1, without fusion to other proteins so that the ELISA measured binding activities specific to PD1. Based on this assay, these antibodies exhibited a wide range of PD1 binding activities, and some of the new anti-PD1 antibodies (e.g. M1-5, M2-1 and M2-3 in FIG. 5C; M1-4, 1-6, M2-5, M1-3, M2-2 in FIG. 8C) outperformed, by far, the 17D8 antibody, Nivolumab and Pembrolizumab (FIG. 5C and FIG. 8C). Two of the strongest new anti-PD1 antibodies (M1-5 and M2-1 in FIG. 5C) were chosen to quantify their PD1 binding affinity with Biacore, in side-by-side comparison with the 17D8 antibody, Nivolumab and Pembrolizumab (FIG. 5D).

FIG. 5C depicts ELISA analysis of PD1-binding activities of 17D8 antibody, Nivolumab, Pembrolizumab and five new anti-PD1 antibodies isolated from Mouse Models 1 and 2. In this ELISA experiment, PD1 extracellular domain, without Fc fusion, was coated on the ELISA plate. The x-axis represents antibody concentration in log 10 scale; the y-axis displays OD405, which correlates with the levels of antibody binding to immobilized PD1. The titration curves were marked with the corresponding antibodies to the right of the plot; the order, from top to bottom, indicates the relative binding activities of the antibodies.

FIG. 5D depicts Biacore analysis of binding kinetics of select anti-PD1 antibodies. In this assay, antibodies were immobilized on sensor chips, and PD1 extracellular domain, the same protein used in the ELISA experiment in FIG. 5C, flowed through sensor chip. The kinetics of PD1/antibody interaction was measured in real time. The table lists the association rate constant (kₐ), dissociation rate constant (k_{d}) and equilibrium dissociation constant (K_{D}) for each antibody.

Contrary to the ELISA results, all five antibodies showed similar PD1 binding affinities, with KD values in the nM range, which is generally considered high affinity antibody/antigen interaction. Biacore analysis is expected to be a more reliable measure of antibody binding affinity than ELISA. In the Biacore assay, PD1 was in solution phase, and all regions of the PD1 molecule should be accessible to antibodies. In contrast, immobilization of PD1 molecules on ELISA plate may occlude certain regions from antibody recognition. Under this condition, antibody binding activity will be influenced by epitope accessibility, and the apparent different PD1 binding activities of the antibodies in ELISA (FIG. 5C) may reflect distinct epitope specificities. For example, antibodies M1-5, M2-1 and M2-3 may target epitopes that are readily accessible on immobilized PD1 molecules in ELISA, whereas the PD1 epitopes for the 17D8 antibody, Nivolumab and Pembrolizumab may be partially obstructed by the ELISA plate.

To test this hypothesis, ELISA plates were coated with PD1-Fc, which was used initially to screen serum response of immunized mouse models and showed robust interaction with anti-PD1 IgG from the plasma (FIGS. 4A-4B). PD1-Fc may attach to ELISA plates in a way that renders PD1 more accessible to antibody interaction. Indeed, with PD1-Fc as the coating antigen, all antibodies showed similar PD1 binding activities in ELISA (FIG. 5E and FIG. 8E). As another assay for PD1 binding activities, the binding of these antibodies to PD1 expressed on the surface of NS1 cell, a mouse plasmacytoma cell line, was tested; this assay provides a more physiological condition for PD1 recognition than ELISA, and all regions of the PD1 extracellular domain should be readily accessible for antibody interaction.

FIG. 5E depicts ELISA analysis of the same antibodies in FIG. 5C, but with PD1-Fc fusion as the coating antigen on ELISA plate. The ELISA plots are labeled the same way as in FIG. 5C. Since the binding curves of all the antibodies largely overlapped in this ELISA experiment, their binding activities are similar in this assay, and the order of antibodies to the right of the plot does not indicate their relative binding activities.

As in the PD1-Fc ELISA (FIG. 5E and FIG. 8E), most of the antibodies analyzed in this experiment showed comparable levels of binding to surface-expressed PD1 (FIG. 5F and FIG. 8F); only two antibodies, M1-7 and M1-2, showed appreciably weaker binding activity than the other antibodies (FIG. 5F and FIG. 8F). FIG. 5F depicts FACS analysis of binding of anti-PD1 antibodies to PD1 expressed on cell surface. The x-axis of the FACS plots represent binding levels of the antibodies; the y-axis of the plots represents cell number.

Thus, the results from the Biacore measurement (FIG. 5D), PD1-Fc ELISA (FIG. 5E and FIG. 8D), and surface PD1-binding assay (FIG. 5F and FIG. 8F) all suggested that the new anti-PD1 antibodies have similar binding affinities to PD1 as the original 17D8 antibody. Nonetheless, these antibodies appear to recognize different PD1 epitopes, which have different levels of accessibility on immobilized PD1 molecule, and the apparent binding activities of the anti-PD1 antibodies in the PD1 ELISA experiment (FIG. 5C) may actually correlate with the accessibility of their respective epitopes on immobilized PD1.

The new anti-PD1 antibodies originated from primary antibodies that had the same CDR H1 and CDR H2 in the 17D8 VH segment. The structure of the 17D8 antibody and PD1 complex has not been reported. However, given the homology between the 17D8 antibody and Nivolumab, the structure of Nivolumab and PD1 complex can serve as a guide to infer the roles of CDRs of 17D8 and related variant antibodies in PD1 interaction. In the case of Nivolumab, the CDR H1 and CDR H2 contact the N-terminal loop of PD1. Thus, the 17D8 VH containing primary antibodies in both PD1 Diversification Mouse Models may be predisposed to interact with the N-terminal loop of PD1.

The main distinction between the new and the original 17D8 antibodies lie in CDR H3. The CDR H3 of Nivolumab, and potentially the homologous CDR H3 of 17D8, interacts with the FG loop of PD1. The unrelated CDR H3s in the new anti-PD1 antibodies may target other regions of PD1. In some cases, the new CDR H3s may have evolved in the context of strengthening the interaction between the N-terminal loop and the CDR H1 and H2 in the 17D8 VH segment; such antibodies would be focused on the N-terminal loop. Since the N-terminal loop is a linear epitope, it may be more accessible than conformational epitopes, such as the epitope for the 17D8 antibody that involves two separate regions of PD1. This difference may underlie the more robust binding activities of certain new antibodies (e.g. M1-5, M2-1 and M2-3) in PD1 ELISA than the 17D8 antibody (FIG. 5C).

To test this hypothesis, the N-terminal loop of PD1 was appended to GST protein and tested the binding of the anti-PD1 antibodies to this fusion protein in ELISA. The new anti-PD1 antibodies exhibited a range of binding activities to the PD1 N-terminal loop-GST fusion (FIG. 5G and FIG. 7E). FIG. 5G depicts ELISA analysis of the same antibodies in FIG. 5C, but with PD1 N-terminal-GST fusion protein as the coating antigen on ELISA plate. The ELISA plots are labeled the same way as in FIG. 5C.

Some antibodies (e.g. M1-5 in FIG. 5G; M1-4, M2-5, M1-6 in FIG. 8E) showed strong binding activity in this ELISA, suggesting that the N-terminal loop may constitute their principle epitope. At the other end of the spectrum, some antibodies showed no detectable binding to the PD1 N-terminal loop-GST fusion protein (e.g. M1-7 in FIG. 5G; M1-2 in FIG. 8E), suggesting that their epitopes may involve other regions of PD1. Pembrolizumab is a representative of this type of antibody (FIG. 5G), as the antibody does not contact the N-terminal loop of PD1. The 17D8 antibody and Nivolumab also bound poorly to the PD1 N-terminal loop-GST fusion (FIG. 5G), because these antibodies require additional regions of PD1 (e.g. the FG loop) for stable interaction.

Between the two extremes, the other antibodies showed intermediate levels of binding to the PD1 N-terminal loop (e.g. M2-1, M2-3 and M1-1 in FIG. 5G; M2-2, M1-3, M2-4 in FIG. 8E); for these antibodies, the N-terminal loop may be part of their epitopes. Examining these antibodies as a whole, the binding to the PD1 N-terminal loop-GST fusion protein correlates generally with the binding to unfused PD1 in ELISA (compare FIG. 5G with 3C and FIG. 8E with 8C), presumably because the N-terminal loop of immobilized PD1 was readily accessible for antibody interaction. This experiment helped to resolve the discrepancies of PD1 binding activities of some antibodies in different assays (FIGS. 5C-5F). More importantly, the comparison of PD1 binding activities of new anti-PD1 antibodies versus the original 17D8 antibody in different assays revealed epitope diversification of the prototype in both Mouse Models.

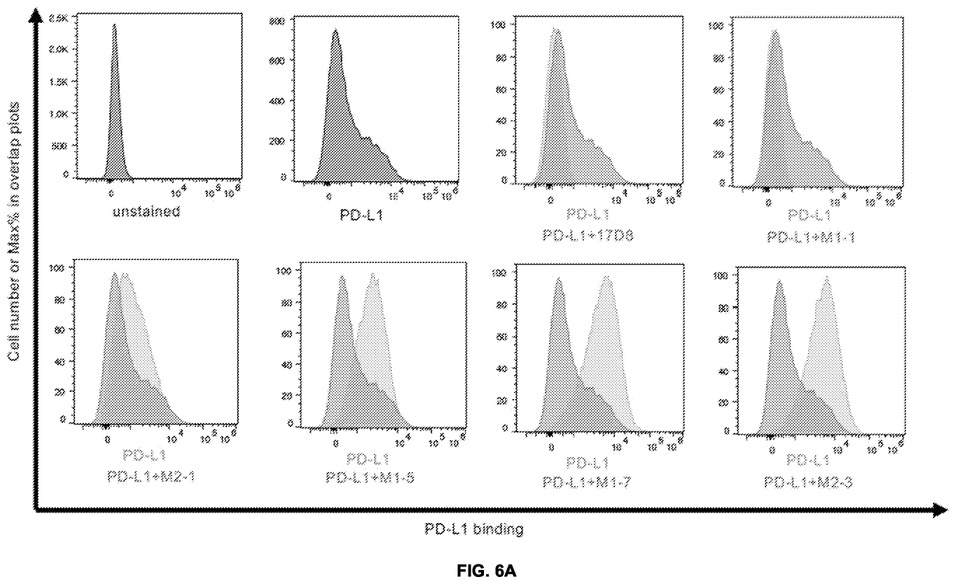

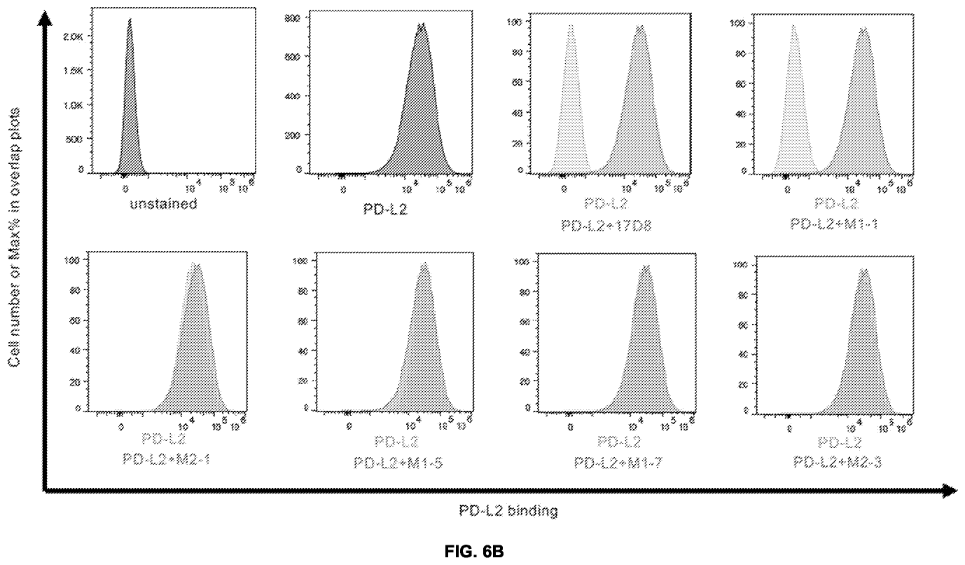

Antibodies targeting different PD1 epitopes may exert distinct effects on PD1 interaction with its ligands. A cell-based assay was used to test this possibility. In this experiment, the binding of the two PD1 ligands, PD-L1 or PD-L2, to PD-1 expressed on the NS1 cell surface was measured. Although both PD-L1 and PD-L2 bound to surface expressed PD1 in this assay, PD-L1 exhibited lower binding activity than PD-L2 (compare PD-L1 in FIG. 6A with PD-L2 in FIG. 6B), in agreement with biochemical measurements of PD1/ligand interaction. As expected from its homology to Nivolumab, addition of the 17D8 antibody abrogated PD1 interaction with PD-L1 (FIG. 6A) or PD-L2 (FIG. 6B). One new anti-PD1 antibody, M1-1, had the same inhibitory effect on PD1/ligand interaction (FIGS. 6A and 6B).

FIG. 6A depicts FACS analysis of the effects of anti-PD1 antibodies on PD1/PD-L1 interaction. The x-axis of the plots represents the levels of PD-L1 binding to PD1 expressed on NS1 cell surface; the y-axis represents relative cell number, with the highest peak set at 100% (modal mode). The addition of PD-L1 and various anti-PD1 antibodies are indicated underneath the plots. In the plot “PD-L1”, only PD-L1 was added to the binding reaction, and this data was used in all the subsequent overlay plots, as represented by the red histogram; the blue histograms show PD-L1 binding to PD1 in the presence of various antibodies. FIG. 6B depicts FACS analysis of the effects of anti-PD1 antibodies on PD1/PD-L2 interaction. The plots in this section is labeled in the same manner as in FIG. 6A.

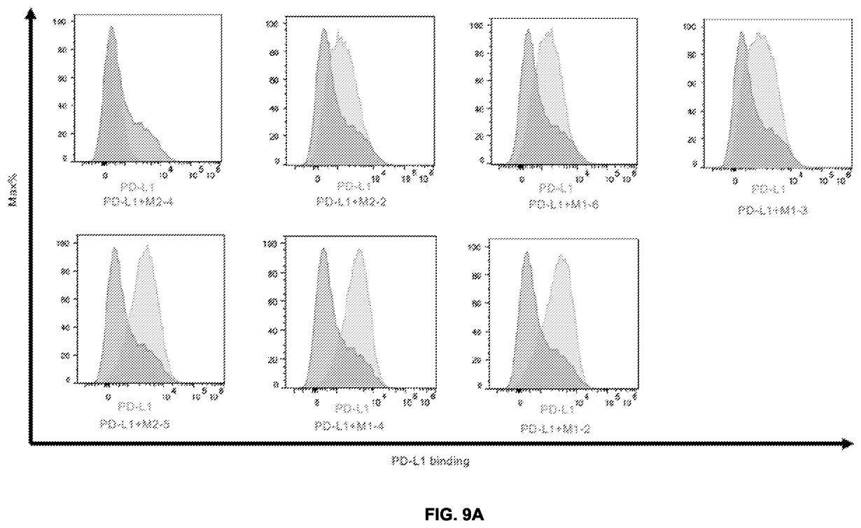

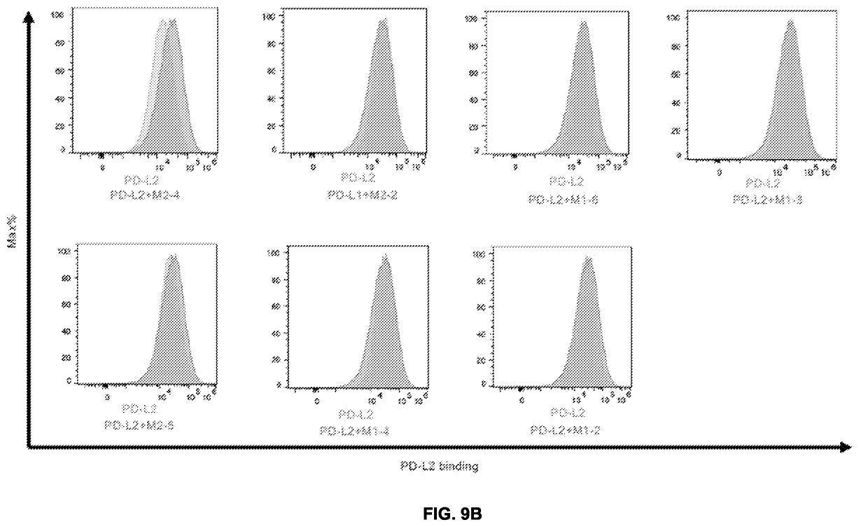

By contrast, the other 4 new antibodies (M2-1, M1-5, M1-7 and M2-3) enhanced PD1/PD-L1 interaction to varying degrees, with antibodies M1-7 and M2-3 exhibiting the strongest effects (FIG. 6A). On the other hand, these 4 antibodies (M2-1, M1-5, M1-7 and M2-3) did not affect PD1/PD-L2 interaction (FIG. 6B). Additional new anti-PD1 antibodies exhibited similar activities: enhancing PD1/PD-L1 interaction but having no effect on PD1/PD-L2 interaction (FIGS. 9A and 9B). One new antibody, M2-4, inhibited PD1/PD-L1 interaction, but had minimal effect on PD1/PD-L2 interaction (FIGS. 9A and 9B). These results showed that diversification of the 17D8 antibody produced variants with different activities: antibody M1-1 can inhibit both PD1/PD-L1 (FIG. 6A) and PD1/PD-L2 (FIG. 6B) interaction, antibody M2-4 can block PD1/PD-L1 (FIG. 9A) but not PD1/PD-L2 (FIG. 9B) interaction, and the other 10 antibodies can stimulate PD1/PD-L1 interaction to varying degrees (FIGS. 6A and 9A) but do not affect PD1/PD-L2 (FIGS. 6B and 9B) interaction.

PD1 blocking antibodies work by a competition mechanism. Based on structural studies, Nivolumab binding to PD1 clashes with PD1/ligand interaction, and the homologous 17D8 antibody likely functions similarly. As shown above, the new anti-PD1 antibody M1-1 also blocked PD1/ligand interaction. Among the 12 new antibodies analyzed in the PD1/ligand interaction assay (FIGS. 6A-6B and FIGS. 9A-9B), the M1-1 antibody was the only one that preserved the CDR H3 of the 17D8 antibody (FIG. 5A). Because of the CDR H3 conservation, the M1-1 antibody may interact with PD1 in a similar manner as the 17D8 antibody to inhibit PD1/ligand interaction. The other new anti-PD1 antibodies contained different CDR H3s from the 17D8 antibody (FIGS. 5A and 5B, FIGS. 7A-7D, FIGS. 8A and 8B).

In addition, some of these new antibodies also accumulated substantial levels of SHM throughout both the IgH and IgL variable regions (FIGS. 5A and 5B, FIGS. 7A-7D, FIGS. 8A and 8B). Altogether, these sequence changes, especially those in CDRs, can alter the way these antibodies contact PD1; for example, some of the new antibodies (e.g. M1-5, M2-1 and M2-3 in FIG. 5G) targeted primarily the N-terminal loop of PD1, whereas the 17D8 antibody contacted additional regions of PD1. The N-terminal loop specificity cannot account for the stimulatory effect of the new anti-PD1 antibodies. One of the best stimulatory antibodies, M1-7 (FIG. 6A), barely associated with the PD1 N-terminal loop in ELISA (FIG. 5G); by contrast, the M2-1 antibody bound strongly to the PD1 N-terminal loop (FIG. 5G), but this antibody had minimal stimulatory effect on PD1/PD-L1 interaction (FIG. 6A).

Structural studies of the antibody/PD1/PD-L1 complex would help to elucidate the mechanism that underlies the stimulatory effect of some of the new anti-PD1 antibodies. In such structural studies, comparison of the M1-7 and M2-3 antibodies can be particularly informative, as these two antibodies showed comparable stimulatory effects on PD1/PD-L1 interaction; but they appeared to target different epitopes in PD1, based on their distinct binding activities to PD1 and PD1 N-terminal loop in ELISA. Another unresolved question is why, with the exception of the M1-1 antibody, none of the new antibodies inhibit PD1/PD-L2 interaction. PD-L1 and PD-L2 bind to PD1 in a similar fashion; however, PD-L2 has higher affinity for PD1 than PD-L1 perhaps rendering the PD1/PD-L2 association less susceptible to modulation by antibodies.

In summary, this example served as a proof-of-concept validation of our antibody diversification strategy to derive variant antibodies from a prototype. Moreover, this study yielded a panel of new anti-PD1 antibodies that deserve further investigation for potential therapeutic applications. For example, among the new anti-PD1 antibodies, M1-7 and M2-3 exerted the most obvious stimulatory effects on PD1/PD-L1 interaction (FIG. 6A). If these in vitro stimulatory effects are translatable to in vivo situations, these antibodies (M1-7 and M2-3) can potentiate PD1-dependent inhibitory pathway to suppress deleterious T cell activities in autoimmune diseases. In this regard, PD1/PD-L1 interaction plays an important role in preventing autoimmunity.

These antibodies do not affect PD1/PD-L2 interaction. PD-L2 is primarily expressed on professional antigen presenting cells, whereas PD-L1 has a broader expression pattern. Thus, these antibodies may not affect T cell priming by professional antigen-presenting cells, and their primary impacts would be on effector T cell activity toward PD-L1 target cells. The specificity can be exploited to dampen autoimmune T cell attack on host tissues, without general immune suppression. These antibodies can also inhibit T cell activity independently of PD-L1. IgG isoforms of the antibodies can attach to cell surface via Fey receptors and engage PD1 directly on T cells to down-regulate T cell activity. All these potential applications will require functional evaluation in vivo.

The genetic modifications for the mouse models were introduced into mouse embryonic stem (ES) cells. For replacement of the mouse V_{H}81X segment with the 17D8 V_{H} segment, a homologous recombination construct was generated, which contains homology arms that flank the mouse V_{H}81X segment. The homologous recombination construct was transfected into a previously established ES cell line that was used to generate a mouse model for testing HIV-1 vaccine candidates. In this ES cell line, the mouse V_{H}81X segment was replaced with the human V_{H}1-2 gene segment. Moreover, the IGCRI regulatory element was deleted to increase the usage of the human V_{H}1-2 or any human gene segment in place of the mouse V_{H}81X segment. After transfection, stable genomic integration of the recombination construct into ES cells was selected with G418, based on a neomycin resistance gene in the construct. The stable clones were screened, with Southern hybridization, for correct integration of the human 17D8 V_{H} segment into the V_{H}81X locus. The neomycin resistance gene was removed via deletion by flanking loxP sites by transduction of the ES clone with Adenovirus-cre. All the genetic modifications involved in the mouse models, including 17D8 DJ_{H} and 17D8 LC KIs, were introduced into the respective loci, as diagramed in FIGS. 1A and 1B, in the same manner. The ES clones with the complete set of genetic modifications for mouse model 1 or model 2 were injected into Rag2 deficient blastocysts to generate chimeric mice. All the immunization experiments in this study were carried out with such chimeric mice.

To determine B cell reconstitution in chimeric mice, blood samples were collected from the chimeric mice. B cells were stained with APC-anti-B220 antibody and FITC-anti-IgM antibody (FIGS. 3A and 3B). The FACS reaction was analyzed on a BD FACS Calibur machine, and the data were plotted with FlowJo software. To analyze the representation of 17D8 HC among B cells in the chimeric mice (FIGS. 3C and 3E), splenic B cells from the mouse models were fused with NS1 plasmacytoma cells to generate hybridomas. V(D)J recombination products involving the 17D8 V_{H} segment was detected by PCR amplification with a primer specific to the 17D8 V_{H} Segment and a primer downstream of the J_{H} region. To verify the expression of 17D8 LC in B cells, RNA was purified from splenocytes of the mouse models. 5′RACE for total Igκ cDNA was initiated with a primer specific to Cκ, and the PCR products were cloned into shuttle vector. Individual clones were sequenced, and all corresponded to the 17D8 LC (FIGS. 3D and 3F).

The immunogen, PD1-GST fusion protein, consisted of the extracellular domain of human PD1 fused at the C-terminus to GST. The fusion protein was expressed in E. coli and purified with glutathione agarose. Each mouse was immunized with 50 μg of PD1-GST protein plus Inject-Alum by intraperitoneal injection. The mouse model 1, from which the 319-9-x antibodies were isolated, was immunized 5 times altogether; the mouse model 2, from which the 397-27-x antibodies were isolated, was immunized 4 times in total.

To monitor the induction of anti-PD1 antibody by immunization, blood samples were collected before and after the second immunization. Plasma was separated from the cellular fraction by centrifugation. IgG concentrations in the plasma was measured with ELISA; in this assay, unlabeled goat anti-mouse IgG antibody was used for capture, alkaline phosphatase (AP) conjugated goat anti-mouse IgG served as secondary antibody for detection, and purified mouse IgG was the standard for quantification. Based on this initial measurement, the plasma concentrations were adjusted to the same IgG concentrations, which were verified in ELISA (FIGS. 4A and 4B). The levels of anti-PD1 IgG in the same plasma samples were measured with ELISA (FIGS. 4A and 4B). For ELISA detection of anti-PD1 antibodies (FIGS. 4A and 4B), ELISA plates were coated with PD1-Fc fusion protein, which is composed of the extracellular domain of human PD1 and the human IgG4 Fc region. The use of PD1-Fc fusion avoided detection of anti-GST antibodies that were induced by PD1-GST immunization. The PD1-Fc fusion was expressed in 293F cells and purified via a C-terminal His tag on Ni-column. The secondary antibody for the ELISA was AP-goat anti-mouse IgG (FIGS. 4A and 4B).

To isolate PD1-specific B cells, spleen was dissected 5 days after the last immunization. IgG⁺ B cells were enriched with a memory B cell purification kit. The IgG⁺ B cell preparation was stained with Alexa488-PD1-mFc, PE-anti-B220 and Sytox blue, and the B cells were already stained with APC-anti-IgG antibodies from the memory B cell purification kit. PD1-mFc fusion protein consisted of the extracellular domain of human PD1 and mouse IgG1 Fc region. The fusion protein was expressed in 293F cells, purified via C-terminal His tag on Ni-column and labeled with Alexa488. The use of PD1-mFc fusion selected for PD1-specific B cells, instead of GST-specific B cells, which should also be present in the IgG⁺ B cell population. PD1-specific IgG+ B cells were sorted as single cells into 96 well plates on FACS Arias (FIGS. 4C and 4D). The Ig HCs and LCs of the single cells were amplified with primers specific for the 17D8 V_{H}/mouse Cγ1, 2a, 2b or 17D8 VW/mouse Cκ, respectively. The cells that were positive for both the 17D8 HC and LC PCRs were counted as expressing 17D8 related antibodies, as shown in the pie chart (FIGS. 4C and 4D). The PCR products for the HC or the LC were sequenced, and their sequences were aligned with the 17D8 HC or LC sequences (FIGS. 7A-7D) with MegaAllign software, and mutation frequency (FIGS. 4G and 4H) was based on substitution ratios of the VH segment. CDR H3 logo plots (FIGS. 4E and 4F) were generated with WebLogo.

For the ELISA experiment in FIG. 5A, anti-PD1 antibodies were produced by transient transfection of expression constructs into 293F cells or Expi293 cells; all antibodies were expressed as human IgG4/κ antibodies. These antibodies contained a 6×His tag (SEQ ID NO: 99) at the C-terminus of the Fc region and were purified from culture supernatant on Ni-column. For measurement of PD1 binding activity, ELISA plates were coated with PD1-Fc fusion protein. Antibodies were added to the plates; in the dilution series, the highest concentration of all the antibodies were 1p g/ml, as confirmed by ELISA quantification of IgG concentration. After washing, antibodies retained on the plates were detected with AP-anti-human kappa antibody. For the ELISA experiment in FIGS. 5A-5G, ELISA plates were coated with PD1 N-terminal loop-GST fusion protein; the N-terminal loop peptide is: LDSPDRPWNP (SEQ ID NO: 100). The N-terminal loop-GST protein was produced in E. coli and purified on glutathione agarose. The same anti-PD1 antibodies as those used for the PD1-Fc fusion ELISA was analyzed in the PD1 N-terminal loop-GST ELISA, and AP-anti-human kappa antibody served as the secondary antibody for detection.

To test the binding of anti-PD1 antibodies to surface expressed PD1, full-length human PD1 in mouse plasmacytoma cell line, NS1, was expressed. As confirmed by FACS staining, the parental NS1 cells did not exhibit appreciable cross-reactivity with any of the anti-PD1 antibodies in this study (FIGS. 8A-8F). A construct that expresses the full length human PD1 was transfected into NS1 cells, and clones with stable integration of the expression construct were obtained. One clone was chosen, referred to as PD1-NS1, for the FACS analysis in FIG. 5B and FIGS. 6A-6B. For the FACS analysis in FIG. 5B, the PD1-NS1 cells were incubated with anti-PD1 antibodies. The binding of the PD1 antibodies were revealed with PE-anti-human kappa antibody. The FACS staining reaction was analyzed on Attune NXT FACS machine, and the data were plotted with FlowJo 10 software.

Biacore analysis of the kinetics of the interaction between anti-PD1 antibodies and PD1 was performed by Affina Biotechnologies. Given the quantitative and sensitive nature of the assay, non-physiological modifications of the antibodies and PD1 were minimized. Thus, the antibodies did not contain C-terminal His tag; these antibodies were expressed as human IgG4/k antibodies in Expi293 cells and purified on Protein A column. The PD1-Fc fusion protein contains a TEV protease cleavage site. After TEV digest, the PD1 portion was separated from the Fc region. Under physiological conditions, PD1 exists as a monomer. However, in the context of the PD-Fc fusion, PD1 is dimerized through the Fc region and may engage in bivalent interactions with IgG antibodies. The avidity effect can cause major deviations from the actual binding affinity between monomeric PD1 and single antigen-binding site. Separation of PD1 from the Fc portion prevented this error. In the biacore assay, the antibodies were immobilized on sensor chips and PD1 was passed over the immobilized antibodies. Association and dissociation of antibodies and PD1 were detected in real time. Based on these data, the on-rate and off-rate of antibody/PD1 interaction were determined, and the KD value was derived by dividing the off-rate with on-rate.

PD-L1 and PD-L2 were expressed as fusion proteins with human IgG4 Fc regions in Expi293 cells, purified from the culture supernatant via C-terminal His tag on Ni-column and biotinylated on Avi-tag at the C-terminus. For PD1/ligand interaction experiments in FIGS. 6A-6B, the biotinylated PD-L1 or PD-L2 were incubated with PD1-NS1 cells with or without anti-PD1 antibodies. After washing, PD-L1 or PD-L2 retained on cell surface were revealed with PE-Streptavidin. The binding reaction was analyzed on Attune NXT flow cytometer, and the data were plotted with Flowjo software.

The sequences encode the Ig variable regions of the new anti-PD1 antibodies that were used in the experiments in FIGS. 5A-5G, FIGS. 6A-6B, FIGS. 8A-8F, and FIGS. 9A-9B. In the expression constructs, the human V_{H}3-33 leader sequence and the human Vk3-11 leader sequence were appended to the 5′ end of the variable region sequences of the HCs and LCs, respectively; human V_{H}3-33 and Vk3-11 segments are germline components of the 17D8 antibody heavy and light chains, respectively. The IgH variable regions were expressed in association with the human IgG4 constant region, and the IgL variable regions were expressed in association with the human kappa constant region. The complete cDNAs for the HCs and LCs were cloned into the pcDNA expression vector and expressed in 293F or Expi293 cells. The Ig variable sequences of the new anti-PD1 antibodies that were used are listed as follows.

M1-1HC
(SEQ ID NO: 101)
CAGGTGCAGTTGGTGGAGTCTGGGGGAGACGTGGTCCAGCCTGGGGGGTCCCTGAGACT
CTCCTGTGCAGCGTCTGGAGTCGCCTTCAGGGACTATGGCATGCACTGGGTCCGCCAGGC
ACCAGGCAAGGGGCTGGAGTGGGTGGCAGTTATATGGTATGATGGAAGTAAGAAATATT
ATGGAGACTCCGTGAAGGGCCGATTCACCGTCTCCAGAGACAATTCCAAGAACATGTTG
TATCTGGAAATGAACGGCCTGAAAGCCGAGGACACGGCAATGTATTATTGTGCGAGGAA
CGATGACTACTGGGGCCAGGGAACCCTGGTCACCGTCTCCTCAG
M1-2HC
(SEQ ID NO: 102)
CAGGTGCAGCTGGTGGAGTCTGGGGGAGACGTGGTCCAGCCGGGGGGGTCCCTGAGACT
CTCCTGTGCGGCGTCTGGAGTCGCCTTCAGTGACTATGGCATGGAATGGGTCCGCCAGGC
TCCAGGCAAGGGGCTGGAGTGGCTGGCAGTTATCTGGTATGATGGAAGTAGGAAACACT
ATGCAGACTCCGTGAAGGGCCGATTCACCATCTCCCGCGACAATTCCAAGAACATTCTGT
ATCTACAAATGAACAGCCTGAGAGCCGAGGACACGGCTATGTATTACTGTGCGAGATGC
CACTCTAAAGATGACTACTGGGGCCAGGGAACCCTGGTCACCGTCTCCTCAG
M1-3HC
(SEQ ID NO: 103)
CAGGAGTGGTTGGTGGAGTCGGGGGGAGACGTGGTCCAGCCGGGGGGGTCCCTGAGACT
CTCCTGTGCGACGTCTAAAGTCACCTTCAATGACTTTGGCATTCACTGGGTCCGCCAGGC
TCCAGGCAAGGGACTGGAGTGGGTGGCAATTATTTGGTATGATGGAAGCAGGAATCACT
ACGCAGACTCCGTGAGGGGCCGATTCACCCTCTCCAGGGACAATTCCAAAAACATGGTC
CATCTTCACATGAGTAGCCTGAGAACCGAGGACACGGCTATGTATTATTGTGCGCGAGG
AATACACTCTAACGATGACTATTGGGGCCAGGGAACCATGGTCACCGTCTCCTCAG
M1-4HC
(SEQ ID NO: 104)
CAGGTTCAGCTGGTGGAATCTGGGGGAGACGTGGTCCAGCCGGGGGGGTCCCTGAGACT
CTCCTGTGCAGTGTCTGGAGTCACCTTCGGTGACTTTGGCTTCGAATGGGTCCGCCAGGC
TCCAGGCAAGGGTCTGGAGTGGGTGGCAGTTATTTGGTACGACGGAAGCAGAAAACATT
ATGCAGAGTCCGTGAGGGGCCGATTTACCATCTCCAGAGACAATTCCAGGAACATGATG
TATCTGGAAATGACTGGACTGAGAGTCGAGGACACGGCTAAATATTACTGTACGAGAAG
TCACTCTCACGAGGACTACTGGGGCCAGGGAACCCTGGTCACCGTCTCCTCAG
M1-5HC
(SEQ ID NO: 105)
CAGGTGCAGCTGGTGGAGTCTGGGGGAGACGTGGTCCAGCCTGGGGGGTCCCTGAGACT
CTCCTGTACAGTGTCTGGAGCCGTCTTCCGTGACTTGGGCATGGAATGGGTCCGTCAGGC
CCCAGGCAAGGGGCTGGAATGGTTGGCAGTTATATGGTACGACGGAGGTAGAAAACACT
ATGCGGACTCCGTGAAGGGCCGGTTCACCATCTCCAGAGACAATTCCAGGAACATGCTCT
TTCTGCAAATGAATGGACTGAGAGTCGACGACACGGCTATGTATTACTGTACGAGAAGC
CACTCTACCGATGATTACTGGGGCCAGGGAACCCTGGTCACCGTCTCCTCAG
M1-6HC
(SEQ ID NO: 106)
CAGGTGCAGTTGGTGGAGTCTGGGGGAGACGTGGTCCAGTCGGGGGGGTCCCTGAGACT
CTCCTGTGCAGTGTCTGGAGTCGTCTTCAGTGATTATGGCTTCGAATGGGTCCGCCAGGC
TCCAGGCAAGGGGCTGGAGTGGGTGGCAGTTATATGGTACGACGGAAGTAGGAAACATT
ATGCAGACTCCGTCCAGGGCCGATTCACCATTTCCAGAGACAATTTCCGGAACATGTTGT
ATCTACAAATGACTGGACTGAGAGTCGAGGACACGGCTAAATACTATTGTACGAGAAGC
CACTCTCACGAGGACTACTGGGGCCAGGGAACCCTGGTCACCGTCTCCTCAG
M1-7HC
(SEQ ID NO: 107)
CAATTACAACTGGTGGAGTCTGGGGGAGACGTGGTCCAGCCTGGGGGGTCCCTGAGACT
CTCCTGTGCGGCGTCTGGGGTCGTCTTCAGTGACTTTGGCCTGGAATGGGTCCGCCAGGC
TCCAGGCAAGGGGCTGGAGTGGCTGGCAGTTATCTGGTATGATGGAAGTCGGAAACATT
ATGCAGACTCCGTGAAGGGCCGATTCACCATCTCCAGGGACAATTCCAAGAACATGCTCT
ATCTGCAAATGAACAGTCTGAGAGTCGAGGACACGGCTATGTACTACTGTGCGCGATGT
CACTCTAAAGAGGACTACTGGGGCCAGGGAACCCTTGTCACCGTCTCCTCAG
M2-1HC
(SEQ D NO: 108)
CAGGTGCAGCTGGTGGAGTCTGGGGGAGACGTGGTCCAGCCTGGGGGGTCCCTGAGACT
CTCCTGTTCAGCGTCTGGACTCGTATTCAGAGACTATGGCATGAACTGGGTCCGCCAGGC
TCCAGGCAAGGGGCTGGAGTGGGTCGGACTTATATGGTATGATGGAACTAAAAAATATT
ATTCAGACTTCGTGAAGGGCCGATTCACCATCTCCAGAGACAATTCCAAGAACATGTTGT
ATCTACAAATGAACAACCTGAGAGCCGAGGACACGGCTATTTATTACTGTGCGAGATTTC
TAATAGGTGCGACGAGGAGGGGCAATGCTATGGACTACTGGGGTCAAGGGACCTCAGTC
ATCGTCTCATCAG
M2-2HC
(SEQ ID NO: 109)
CAGGTGCAGTTGGTGGAGTCTGGGGGAGACGTGGTCCAGCCTGGGGGGTCCCTGAGACT
CTCCTGTTCAGCGTCTGGGCTCGTAATCAGTGACTATGGCATGAACTGGGTCCGCCAGGC
TCCAGGCAAGGGGCTGGAGTGGGTCGGACTTATATGGTATGATGGAAGTAAAAAATATT
ACTCAGACTTCGTGAAGGGCCGATTCACCATCTCCAGAGACAATTCCAAGAACATATTGT
ATCTACAAATGAACAACCTGAGAGCCGAGGACACGGCTATGTATTACTGTGCGAGATTT
CTAATAGGTGCGACGAGGAGGGGCAATGCTATGGACTATTGGGGTCAAGGAACCTCAGT
CACCGTCTCATCAG
M2-3HC
(SEQ ID NO: 110)
CAGGGGCACCTGGTGGAGTCTGGGGGAGACGTGGTCCTGCCTGGGGGGTCCCTGAGACT
CTCCTGTACAGAGTCTGGTGTCGACCTCAGTGACTTTGGCATACATTGGGTCCGCCAGAC
TCCAGGCAAGGGTCTGGAGTGGGTGGCACTTATCTGGTATGATGGAAGTAAAAAATTTT
ATGCTGACTCCGTGAAGGACCGATTCACCATTTCCAGAGACAATTCCAAGAATATGGTGT
ATCTGGAAATGATCAGCCTGAGAGTCGAGGATACGGCTATGTACTTCTGTGCGAGAGGG
ATACGACGGGGGCCCTGGTTCACTTACTGGGGCCCAGGGACTCTGGTTACAGTCTCTACA
G
M2-4HC
(SEQ ID NO: 111)
CAGGTGCAGTTGGTGGAGTCTGGGGGAGACGTGGTCCAGCCGGGGGGGTCCCTGAGACT
CTCCTGTGCAGCGTCTGGAGTCGCCTTCAGGAACTATGGCATGCACTGGGTCCGCCAGGC
TCCAGGCAAGGGCCTGGAGTGGGTGGCAATTATATGGTATGATGGAAGTAATAAATATT
ATGCAGACTCCGTGAAGGGCCGCTTCACCATCTCCAGAGACAATTCCAAGAATATGTTGT
ATCTTCAAATGAATAGCCTGAGAGCCGAGGACACGGCTATGTATTACTGTGCGAGACTCT
CTATAGGTACGACCCATTACTTTGATACGGACGACTACTGGGGTCAAGGAACCTCAGTCA
CCGTCTCCTCAG
M2-5HC
(SEQ ID NO: 112)
CAGGTGCAACTGGTGGAGTCTGGGGGAGACGTGGTCCAGCCGGGGGGGTCCCTGAGACT
CTCCTGTGCAGCGTCTGGAGTCGTCTTCAGTGACTATGGCTTGTATTGGGTCCGCCAGGC
TCCAGGCAAGGGCCTGGAGTGGGTGGCCCTTATATGGTATGATGGGAGTAAGAAATTTT
ATGCTGACTCCGTGAAGGGCCGATTCTCCATCTCCAGAGACAATTCCAAGAACATGTTGT
ATTTACAAATGAATAATTTGAGAGCCGACGACTCGGCTATTTATTATTGTTCGAGAGGGA
TACGACAGGGGCCATGGTTTGCTTACTGGGGCCAAGGGACTCGTGTCACTGTCTCTCCAG
M1-1LC
(SEQ ID NO: 113)
GAAATTGTGTTGACACAGTCTCCAGCCACCCTGTCTTTGTCTCCAGGGGAAAGAGCCACC
CTCTCCTGCAGGGCCAGTCAGAGTGTTAGCAACTCCTTATCCTGGTACCAACAGAACCCT
GGCCAGTCTCCCAGGCTCATCATCTATGATACATCCAAGAGGGCCACTGGCATCCCAGCC
AGGTTCAGTGGCAGTGGATCTGGGACAGACTTCACTCTCACCATCAACAATCTAGAGACT
GAAGATTTTGCAGTTTATTACTGTCACCAGCGTAGCGACTGGCCTCTCACTTTCGGCGGA
GGGACCAAGGTGGAGATCAAAC
M1-2LC
(SEQ ID NO: 114)
GAAATTGTGTTGACACAGTTTCCGGCCACCCTGTCTCTGTCTCCCGGGGAAAGAGCCACC
CTCTCCTGCAGGACCAGTCAGAATATTGACAGCGACTTAGCCTGGTTCCAACAGAAACCT
GGCCAGGCTCCCAGGCTCATCATCTATGATGCATCCAACAGGGCCACTGGCATCCCAGCC
AGGTTCAGTGGCGGTGGGTCTGGGACAGACTTCACTCTCACCATCACCAGCCTAGAGCCT
GAAGATTTTGCAGTTTATTACTGTCAGCAGCGTACCACCTGGCCTCTCACTTTCGGCGGA
GGGACCAAGGTGGAGATCAAAC
M1-3LC
(SEQ ID NO: 115)
GAAATTGTGTTGACACAGTCTCCAGCCACCCTGTCTTTGTCTCCAGGGGAAAGAGCCACC
CTCTCCTGCCGGACCAGTCAGAGTGTTAGCAGCGACTTAGCCTGGTTCCAACAGAAACCT
GGCCAGGCTCCCAGGCTCTTCATATTTGATGCATCCAAAAGGGTCAATGGCATCCCAGCC
AGGTTCAGTGGCAGTGGGTCTGGGACAGACTTCACTCTCACCATCAGCAGCCTGGAACCT
GAAGATTTTGCAGTTTATTATTGTCAGCAACGTACCGACTGGCCTCTCACTTTCGGCGGA
GGGTCCAGGGTGGAGATCAAAC
M1-4LC
(SEQ ID NO: 116)
GAAATTGTGTTGACACAGTCTCCAGCCACCCTGTCTTTGTCTCCAGGGGAAAGAGCCACC
CTCTCCTGTAGGGCCAGTCAGAGTATTAGCAACTACTTAGCCTGGTTCCAACAGAAATCT
GGCCAGGCTCCCAGGCTCATCATCCATGATGCATTTAAACGGGCCGCTGGCATCCCAACC
AGGTTCAGTGGCAGTGGGTCTGGGACAGACTTCACTCTCACCATCAGCAGTCTAGAGCCT
GAAGATTTTGCAGTTTATTATTGTCAGCAGCGTGACAACTGGCCTCTCAATTTCGGCGGA
GGGACTAAGGTGGAGATCAAAC
M1-5LC
(SEQ ID NO: 117)
GAAATTGTGTTGACACAGTCGCCAGCCACTCTGTCTGTGTCTCCAGGGGAAAGAGCCACC
CTCTCCTGTAGGGCCAGTCAGAGTATTAGCAGCGACTTAGCCTGGTTCCAACAGAAACCT
GGCCAGGCTCCCAGGCTCATCATCCATGGTGCATCCAAAAGGGCCACTGGCATCCCAGC
CAGGTTCAGTGGCAGTGGGTCTGGGACAGACTTCACTCTCACCATCAGCAGCCTAGAGCC
TGAAGATTTTGCGGTTTATTACTGTCAGCAGCGTGACAGCTGGCCTCTCAATTTCGGCGG
AGGGACCAAGGTGGAGATCAAAC
M1-6LC
(SEQ ID NO: 118)
GAAATTGTGTTGACACAGTCTCCAGCCACCCTGTCTTTGTCTCCAGGGGAAAGAGCCACC
CTCTCCTGTGGGGCCAGTCAGAATATTGACAACTCCTTAGCCTGGTTCCAACAGAAACCT
GGCCAGGCTCCCAGGCTCATCATCTATGATGCATCTAAAAGGGCCACTGGCATCCCAGCC
AGGTTCAGTGGCAGTGGGTCTGGGACAGACTTCACTCTCACCATCAGCACTCTAGAGCCT
GAAGATTTTGCAGTTTATTATTGTCAGCAGCGTGACCATTGGCCTCTCAATTTCGGCGGA
GGGACCAAGGTGGAAGTCAAAC
M1-7LC
(SEQ ID NO: 119)
GAAATTGAAGTGACACAGTCTCCGGCCACCCTGTCCTTGTCTCCAGGGGAAAGAGCCAC
CCTCTCCTGTAGGGCCAGTCAGAGTATTGACACCGACTTAGCCTGGTTCCAGCAGAGACC
TGGCCAGACTCCCAGACTCATCATCTATGATGCATCCAAAAGGGCCACTGGCATCCCAGC
CAGGTTCAGTGGCGGTGGGTCTGGGACAGACTTCACTCTCACCATCAGCAGCCTAGAGCC
TGAAGATTTTGCAGTTTACTACTGTCAGCAGCGTACCACCTGGCCTCTCACTTTCGGCGG
AGGGACCAAGGTGGAGATCAAAC
M2-1LC
(SEQ ID NO: 120)
GAAGTTGTGTTGACACAGTCTCCAGCCACCCTGTCTTTGTCTCCAGGGGAAAGAGCCACC
CTCTCCTGCAGGGCCAGTCAGAGTATTGACAGCGACTTAGCCTGGTCCCAACAGAAAACT
GGCCAGCCTCCCAGACTCATCATCTATGATGCATCCAACAGGGCCACTGGCATCCCAGCC
AGGTTCAGTGGCAGTGGGTCTGGGACAGACTTCACTCTCACCATCAGTAGTCTAGAGCCT
GAAGATTTTGCAGTTTATTATTGTCAGCAACGTAGCGACTGGCCTCTCACTTTCGGCGGA
GGGACCAAGGTGGAGATCAGAC
M2-2LC
(SEQ ID NO: 121)
GAAGTTGTGTTGACACAGTCTCCAGCCACCCTGTCTTTGTCTCCAGGGGAAAGAGCCACC
CTCTCCTGCAGGGCCAGTCAGAGTATTGACAGCGACTTAGCCTGGTCCCAACAGAAACCT
GGCCAGCCTCCCAGACTCATCATCTATGATGCATCCAACAGGGCCACTGGCATCCCAGCC
AGGTTCAGTGGCAGTGGGTCTGGGACAGACTTCACTCTCACCATCAGCAGCCTAGAGCCT
GAAGATTTTGCAGTTTATTATTGTCAGCAACGTAGCGACTGGCCTCTCACTTTCGGCGGA
GGGACCAAGGTGGAGATCAGAC
M2-3LC
(SEQ ID NO: 122)
GAAATTGTGTTGACACAGTCTCCAGTCATCCTGTCTTTGTCTCCAGGGGAAAGAGCCACC
CTCTCCTGCAGGGCCAGTCAGAGTATTAGCAGCGACTTGGCCTGGTTCCAACAGACACCT
GGCCAGGCTCCCAGGCTCATCATCTATGATGCATCCAACAGGGCCACTGGCATCCCAGCC
AGGTTCAGTGGCAGTGGGTCTGGGACAGACTTCACTCTCACCATCAGCAGCCTAGAGCCT
GAAGATTTTGCAGTTTATTACTGTCAGCAGCGGAGCAACTGGCCTCTCACTTTCGGCGGA
GGGACCAAGGTGGAGATCAAAC
M2-4LC
(SEQ ID NO: 123)
GAAATTGTGTTGACACAGTCTCCAGCCACCCTGTCTTTGTCTCCAGGGGAAAGAGCCACC
CTCTCCTGCAGGGCCAGTCAGAGTGTTAGCAGCTACTTAGCCTGGTACCAACAGAAGGTT
GGCCAGGCTCCCAGGCTCATCATCTTTGATGCATCCAACAGGGCCACTGGCATCCCAGCC
AGGTTCAGTGGCAGTGGGTCTGGGACAGACTTCACTCTCACCATCACCAGCCTAGATCCT
GAAGATTTTGCAGTTTATTACTGTCAGCAACGTAGCGCCTGGCCTCTCACTTTCGGCGGA
GGGACCAAGGTGGAGATCAGAC
M2-5LC
(SEQ ID NO: 124)
GAAATTGTGCTGACACAGTCTCCAGCCACCCTGTCTTTGTCTCCAGGGGAAAGAGCCACC
CTCTCCTGCAGGGCCAGTCAGAGCATTAGCAGCGACTTAACCTGGTTCCAACAGAAACCT
GGCCAGGCTCCCAGGCTCATCATCTATGATGCATCCAACAGGGCCACTGGCATCCCAGCC
AGGTTCAGTGGCAGTGGGTCTGGGACAGACTTCACTCTCACCATCAGTAGCCTCGAGCCT
GAAGATTTTGTAGTTTATTACTGTCTGCAACGTAGCGACTGGCCTCTCACTTTCGGCGGA
GGGACCAAGGTGGAGATCAAAC

Antibody sequences. The protein sequences of variable regions, heavy chain (HC), and light chain (LC) for the antibodies are listed below in Table 1:

| | |
|---|---|
| Sequence | SEQ ID |

Antibody: 397-27-25 (M2-3)

| | | |
|---|---|---|
| Heavy Chain Variable | QGHLVESGGDVVLPGGSLRLSCTESGVDLSDFGIHWVR | 1 |
| Region | QTPGKGLEWVALIWYDGSKKFYADSVKDRFTISRDNSK | |
| | NMVYLEMISLRVEDTAMYFCARGIRRGPWFTYWGPGT | |
| | LVTVST | |
| Light Chain Variable | EIVLTQSPVILSLSPGERATLSCRASQSISSDLAWFQQTPG | 2 |
| Region | QAPRLIIYDASNRATGIPARFSGSGSGTDFTLTISSLEPED | |
| | FAVYYCQQRSNWPLTFGGGTKVEIK | |
| Heavy Chain CDR1 | GVDLSDFG | 3 |
| Heavy Chain CDR2 | IWYDGSKK | 4 |
| Heavy Chain CDR2 | LIWYDGSKKF | 89 |
| Heavy Chain CDR3 | ARGIRRGPWFTY | 5 |
| Light Chain CDR1 | QSISSD | 6 |
| Light Chain CDR2 | DAS | |
| Light Chain CDR3 | QQRSNWPLT | 8 |

Antibody: 319-9-75 (M1-7)

| | | |
|---|---|---|
| Heavy Chain Variable | QLQLVESGGDVVQPGGSLRLSCAASGVVFSDFGLEWVR | 9 |
| Region | QAPGKGLEWLAVIWYDGSRKHYADSVKGRFTISRDNSK | |
| | NMLYLQMNSLRVEDTAMYYCARCHSKEDYWGQGTLV | |
| | TVSS | |
| Light Chain Variable | EIEVTQSPATLSLSPGERATLSCRASQSIDTDLAWFQQRP | 10 |
| Region | GQTPRLIIYDASKRATGIPARFSGGGSGTDFTLTISSLEPE | |
| | DFAVYYCQQRTTWPLTFGGGTKVEIK | |
| Heavy Chain CDR1 | GVVFSDFG | 11 |
| Heavy Chain CDR2 | IWYDGSRK | 12 |
| Heavy Chain CDR2 | VIWYDGSRKH | 90 |
| Heavy Chain CDR3 | ARCHSKEDY | 13 |
| Light Chain CDR1 | QSIDTD | 14 |
| Light Chain CDR2 | DAS | |
| Light Chain CDR3 | QQRTTWPLT | 16 |

Antibody: 319-9-15 (M1-2)

| | | |
|---|---|---|
| Heavy Chain Variable | QVQLVESGGDVVQPGGSLRLSCAASGVAFSDYGMEWV | 17 |
| Region | RQAPGKGLEWLAVIWYDGSRKHYADSVKGRFTISRDNS | |
| | KNILYLQMNSLRAEDTAMYYCARCHSKDDYWGQGTLV | |
| | TVSS | |
| Light Chain Variable | EIVLTQFPATLSLSPGERATLSCRTSQNIDSDLAWFQQKP | 18 |
| Region | GQAPRLIIYDASNRATGIPARFSGGGSGTDFTLTITSLEPE | |
| | DFAVYYCQQRTTWPLTFGGGTKVEIK | |
| Heavy Chain CDR1 | GVAFSDYG | 19 |
| Heavy Chain CDR2 | VIWYDGSRKH | 20 |
| Heavy Chain CDR3 | ARCHSKDDY | 21 |
| Light Chain CDR1 | QNIDSD | 22 |
| Light Chain CDR2 | DAS | |
| Light Chain CDR3 | QQRTTWPLT | 24 |

Antibody: 319-9-27 (M1-4)

| | | |
|---|---|---|
| Heavy Chain Variable | QVQLVESGGDVVQPGGSLRLSCAVSGVTFGDFGFEWVR | 25 |
| Region | QAPGKGLEWVAVIWYDGSRKHYAESVRGRFTISRDNSR | |
| | NMMYLEMTGLRVEDTAKYYCTRSHSHEDYWGQGTLV | |
| | TVSS | |
| Light Chain Variable | EIVLTQSPATLSLSPGERATLSCRASQSISNYLAWFQQKS | 26 |
| Region | GQAPRLIIHDAFKRAAGIPTRFSGSGSGTDFTLTISSLEPE | |
| | DFAVYYCQQRDNWPLNFGGGTKVEIK | |
| Heavy Chain CDR1 | GVTFGDFG | 27 |
| Heavy Chain CDR2 | IWYDGSRK | 28 |
| Heavy Chain CDR2 | VIWYDGSRKH | 91 |
| Heavy Chain CDR3 | TRSHSHEDY | 29 |
| Light Chain CDR1 | QSISNY | 30 |
| Light Chain CDR2 | DAF | |
| Light Chain CDR3 | QQRDNWPLN | 32 |

Antibody: 397-27-93 (M2-5)

| | | |
|---|---|---|
| Heavy Chain Variable | QVQLVESGGDVVQPGGSLRLSCAASGVVFSDYGLYWV | 33 |
| Region | RQAPGKGLEWVALIWYDGSKKFYADSVKGRFSISRDNS | |
| | KNMLYLQMNNLRADDSAIYYCSRGIRQGPWFAYWGQG | |
| | TRVTVSP | |
| Light Chain Variable | EIVLTQSPATLSLSPGERATLSCRASQSISSDLTWFQQKP | 34 |
| Region | GQAPRLIIYDASNRATGIPARFSGSGSGTDFTLTISSLEPE | |
| | DFVVYYCLQRSDWPLTFGGGTKVEIK | |
| Heavy Chain CDR1 | GVVFSDYG | 35 |
| Heavy Chain CDR2 | IWYDGSKK | 36 |
| Heavy Chain CDR2 | LIWYDGSKKF | 92 |
| Heavy Chain CDR3 | SRGIRQGPWFAY | 37 |
| Light Chain CDR1 | QSISSD | 38 |
| Light Chain CDR2 | DAS | |
| Light Chain CDR3 | LQRSDWPLT | 40 |

Antibody: 319-9-34 (M1-5)

| | | |
|---|---|---|
| Heavy Chain Variable | QVQLVESGGDVVQPGGSLRLSCTVSGAVFRDLGMEWV | 41 |
| Region | RQAPGKGLEWLAVIWYDGGRKHYADSVKGRFTISRDN | |
| | SRNMLFLQMNGLRVDDTAMYYCTRSHSTDDYWGQGT | |
| | LVTVSS | |
| Light Chain Variable | EIVLTQSPATLSVSPGERATLSCRASQSISSDLAWFQQKP | 42 |
| Region | GQAPRLIIHGASKRATGIPARFSGSGSGTDFTLTISSLEPE | |
| | DFAVYYCQQRDSWPLNFGGGTKVEIK | |
| Heavy Chain CDR1 | GAVFRDLG | 43 |
| Heavy Chain CDR2 | IWYDGGRK | 44 |
| Heavy Chain CDR2 | VIWYDGGRKH | 93 |
| Heavy Chain CDR3 | TRSHSTDDY | 45 |
| Light Chain CDR1 | QSISSD | 46 |
| Light Chain CDR2 | GAS | |
| Light Chain CDR3 | QQRDSWPLN | 48 |

Antibody: 319-9-17 (M1-3)

| | | |
|---|---|---|
| Heavy Chain Variable | QEWLVESGGDVVQPGGSLRLSCATSKVTFNDFGIHWVR | 49 |
| Region | QAPGKGLEWVAIIWYDGSRNHYADSVRGRFTLSRDNSK | |
| | NMVHLHMSSLRTEDTAMYYCARGIHSNDDYWGQGTM | |
| | VTVSS | |
| Light Chain Variable | EIVLTQSPATLSLSPGERATLSCRTSQSVSSDLAWFQQKP | 50 |
| Region | GQAPRLFIFDASKRVNGIPARFSGSGSGTDFTLTISSLEPE | |
| | DFAVYYCQQRTDWPLTFGGGSRVEIK | |
| Heavy Chain CDR1 | KVTFNDFG | 51 |
| Heavy Chain CDR2 | IWYDGSRN | 52 |
| Heavy Chain CDR2 | IIWYDGSRNH | 94 |
| Heavy Chain CDR3 | ARGIHSNDDY | 53 |
| Light Chain CDR1 | QSVSSD | 54 |
| Light Chain CDR2 | DAS | |
| Light Chain CDR3 | QQRTDWPLT | 56 |

Antibody: 319-9-53 (M1-6)

| | | |
|---|---|---|
| Heavy Chain Variable | QVQLVESGGDVVQSGGSLRLSCAVSGVVFSDYGFEWV | 57 |
| Region | RQAPGKGLEWVAVIWYDGSRKHYADSVQGRFTISRDNF | |
| | RNMLYLQMTGLRVEDTAKYYCTRSHSHEDYWGQGTL | |
| | VTVSS | |
| Light Chain Variable | EIVLTQSPATLSLSPGERATLSCGASQNIDNSLAWFQQKP | 58 |
| Region | GQAPRLIIYDASKRATGIPARFSGSGSGTDFTLTISTLEPE | |
| | DFAVYYCQQRDHWPLNFGGGTKVEVK | |
| Heavy Chain CDR1 | GVVFSDYG | 59 |
| Heavy Chain CDR2 | IWYDGSRK | 60 |
| Heavy Chain CDR2 | VIWYDGSRKH | 95 |
| Heavy Chain CDR3 | TRSHSHEDY | 61 |
| Light Chain CDR1 | QNIDNS | 62 |
| Light Chain CDR2 | DAS | |
| Light Chain CDR3 | QQRDHWPLN | 64 |

Antibody: 397-27-23 (M2-2)

| | | |
|---|---|---|
| Heavy Chain Variable | QVQLVESGGDVVQPGGSLRLSCSASGLVISDYGMNWVR | 65 |
| Region | QAPGKGLEWVGLIWYDGSKKYYSDFVKGRFTISRDNSK | |
| | NILYLQMNNLRAEDTAMYYCARFLIGATRRGNAMDYW | |
| | GQGTSVTVSS | |
| Light Chain Variable | EVVLTQSPATLSLSPGERATLSCRASQSIDSDLAWSQQK | 66 |
| Region | PGQPPRLIIYDASNRATGIPARFSGSGSGTDFTLTISSLEPE | |
| | DFAVYYCQQRSDWPLTFGGGTKVEIR | |
| Heavy Chain CDR1 | GLVISDYG | 67 |
| Heavy Chain CDR2 | IWYDGSKK | 68 |
| Heavy Chain CDR2 | LIWYDGSKKY | 96 |
| Heavy Chain CDR3 | ARFLIGATRRGNAMDY | 69 |
| Light Chain CDR1 | QSIDSD | 70 |
| Light Chain CDR2 | DAS | |
| Light Chain CDR3 | QQRSDWPLT | 72 |

Antibody: 397-27-3 (M2-1)

| | | |
|---|---|---|
| Heavy Chain Variable | QVQLVESGGDVVQPGGSLRLSCSASGLVFRDYGMNWV | 73 |
| Region | RQAPGKGLEWVGLIWYDGTKKYYSDFVKGRFTISRDNS | |
| | KNMLYLQMNNLRAEDTAIYYCARFLIGATRRGNAMDY | |
| | WGQGTSVIVSS | |
| Light Chain Variable | EVVLTQSPATLSLSPGERATLSCRASQSIDSDLAWSQQK | 74 |
| Region | TGQPPRLIIYDASNRATGIPARFSGSGSGTDFTLTISSLEPE | |
| | DFAVYYCQQRSDWPLTFGGGTKVEIR | |
| Heavy Chain CDR1 | GLVFRDYG | 75 |
| Heavy Chain CDR2 | IWYDGTKK | 76 |
| Heavy Chain CDR2 | LIWYDGTKKY | 97 |
| Heavy Chain CDR3 | ARFLIGATRRGNAMDY | 77 |
| Light Chain CDR1 | QSIDSD | 78 |
| Light Chain CDR2 | DAS | |
| Light Chain CDR3 | QQRSDWPLT | 80 |

Antibody: 319-9-8 (M1-1)

| | | |
|---|---|---|
| Heavy Chain Variable | QVQLVESGGDVVQPGGSLRLSCAASGVAFRDYGMHWV | 81 |
| Region | RQAPGKGLEWVAVIWYDGSKKYYGDSVKGRFTVSRDN | |
| | SKNMLYLEMNGLKAEDTAMYYCARNDDYWGQGTLVT | |
| | VSS | |
| Light Chain Variable | EIVLTQSPATLSLSPGERATLSCRASQSVSNSLSWYQQNP | 82 |
| Region | GQSPRLIIYDTSKRATGIPARFSGSGSGTDFTLTINNLETE | |
| | DFAVYYCHQRSDWPLTFGGGTKVEIK | |
| Heavy Chain CDR1 | GVAFRDYG | 83 |
| Heavy Chain CDR2 | VIWYDGSKKY | 84 |
| Heavy Chain CDR3 | ARNDDY | 85 |
| Light Chain CDR1 | QSVSNS | 86 |
| Light Chain CDR2 | DTS | |
| Light Chain CDR3 | HQRSDWPLT | 88 |

1. An antibody, antibody reagent, antigen-binding fragment thereof, or chimeric antigen receptor (CAR), that specifically binds an PD1 polypeptide, said antibody reagent, antigen-binding portion thereof, or CAR comprising six complementarity determining regions (CDRs) comprising:
(a) a heavy chain CDR1 having the amino acid sequence of SEQ ID NO: 3;
(b) a heavy chain CDR2 having the amino acid sequence of SEQ ID NO: 4;
(c) a heavy chain CDR3 having the amino acid sequence of SEQ ID NO: 5;
(d) a light chain CDR1 having the amino acid sequence of SEQ ID NO: 6;
(e) a light chain CDR2 having the amino acid sequence of SEQ ID NO: 7; and
(f) a light chain CDR3 having the amino acid sequence of SEQ ID NO: 8; or
six complementarity determining regions (CDRs) comprising:
(a) a heavy chain CDR1 having the amino acid sequence of SEQ ID NO: 19;
(b) a heavy chain CDR2 having the amino acid sequence of SEQ ID NO: 20;
(c) a heavy chain CDR3 having the amino acid sequence of SEQ ID NO: 21;
(d) a light chain CDR1 having the amino acid sequence of SEQ ID NO: 22;
(e) a light chain CDR2 having the amino acid sequence of SEQ ID NO: 23; and
(f) a light chain CDR3 having the amino acid sequence of SEQ ID NO: 24.

2.-9. (canceled)

10. The antibody, antibody reagent, antigen-binding portion thereof, or CAR of claim 1, comprising the heavy chain variable region sequence of SEQ ID NO: 1 or 17 or comprising the light chain variable region sequence of SEQ ID NO: 2 or 18.

11. (canceled)

12. The antibody, antibody reagent, antigen-binding portion thereof, or CAR of claim 1, comprising:
the heavy chain variable region sequence of SEQ ID NO: 1 and the light chain variable region sequence of SEQ ID NO: 2; or
the heavy chain variable region sequence of SEQ ID NO: 17 and the light chain variable region sequence of SEQ ID NO: 18.

13. The antibody, antibody reagent, antigen-binding portion thereof, or CAR of claim 12, further comprising a conservative substitution in a sequence not comprised by a CDR.

14. The antibody, antibody reagent, antigen-binding portion thereof, or CAR of claim 1, wherein the antibody reagent or antigen-binding fragment thereof is fully human or fully humanized.

15. The antibody, antibody reagent, antigen-binding portion thereof, or CAR of claim 1, wherein the antibody reagent or antigen-binding fragment thereof is fully humanized except for the CDR sequences.

16. The antibody, antibody reagent, antigen-binding portion thereof, or CAR of claim 1, wherein the antibody reagent or antigen-binding fragment is selected from the group consisting of:
an immunoglobulin molecule, a monoclonal antibody, a chimeric antibody, a CDR-grafted antibody, a humanized antibody, a Fab, a Fab′, a F(ab′)2, a Fv, a disulfide linked Fv, a scFv, a single domain antibody, a diabody, a multispecific antibody, a dual specific antibody, an anti-idiotypic antibody, and a bispecific antibody.

17. A composition, kit, or combination comprising:
(i) the antibody, antibody reagent, antigen-binding portion thereof, or CAR of claim 1 or an antibody, antibody reagent, antigen-binding portion thereof, or CAR comprising six complementarity determining regions (CDRs) comprising:
(a) a heavy chain CDR1 having the amino acid sequence of SEQ ID NO: 11;
(b) a heavy chain CDR2 having the amino acid sequence of SEQ ID NO: 12;
(c) a heavy chain CDR3 having the amino acid sequence of SEQ ID NO: 13;
(d) a light chain CDR1 having the amino acid sequence of SEQ ID NO: 14;
(e) a light chain CDR2 having the amino acid sequence of SEQ ID NO: 15; and
(f) a light chain CDR3 having the amino acid sequence of SEQ ID NO: 16, or
six complementarity determining regions (CDRs) comprising:
(a) a heavy chain CDR1 having the amino acid sequence of SEQ ID NO: 27;
(b) a heavy chain CDR2 having the amino acid sequence of SEQ ID NO: 28;
(c) a heavy chain CDR3 having the amino acid sequence of SEQ ID NO: 29;
(d) a light chain CDR1 having the amino acid sequence of SEQ ID NO: 30;
(e) a light chain CDR2 having the amino acid sequence of SEQ ID NO: 31; and
(f) a light chain CDR3 having the amino acid sequence of SEQ ID NO: 32, or
six complementarity determining regions (CDRs) comprising:
(a) a heavy chain CDR1 having the amino acid sequence of SEQ ID NO: 35;
(b) a heavy chain CDR2 having the amino acid sequence of SEQ ID NO: 36;
(c) a heavy chain CDR3 having the amino acid sequence of SEQ ID NO: 37;
(d) a light chain CDR1 having the amino acid sequence of SEQ ID NO: 38;
(e) a light chain CDR2 having the amino acid sequence of SEQ ID NO: 39; and
(f) a light chain CDR3 having the amino acid sequence of SEQ ID NO: 40, or
six complementarity determining regions (CDRs) comprising:
(a) a heavy chain CDR1 having the amino acid sequence of SEQ ID NO: 43;
(b) a heavy chain CDR2 having the amino acid sequence of SEQ ID NO: 44;
(c) a heavy chain CDR3 having the amino acid sequence of SEQ ID NO: 45;
(d) a light chain CDR1 having the amino acid sequence of SEQ ID NO: 46;
(e) a light chain CDR2 having the amino acid sequence of SEQ ID NO: 47; and
(f) a light chain CDR3 having the amino acid sequence of SEQ ID NO: 48, or
six complementarity determining regions (CDRs) comprising:
(a) a heavy chain CDR1 having the amino acid sequence of SEQ ID NO: 51;
(b) a heavy chain CDR2 having the amino acid sequence of SEQ ID NO: 52;
(c) a heavy chain CDR3 having the amino acid sequence of SEQ ID NO: 53;
(d) a light chain CDR1 having the amino acid sequence of SEQ ID NO: 54;
(e) a light chain CDR2 having the amino acid sequence of SEQ ID NO: 55; and
(f) a light chain CDR3 having the amino acid sequence of SEQ ID NO: 56, or
six complementarity determining regions (CDRs) comprising:
(a) a heavy chain CDR1 having the amino acid sequence of SEQ ID NO: 59;
(b) a heavy chain CDR2 having the amino acid sequence of SEQ ID NO: 60;
(c) a heavy chain CDR3 having the amino acid sequence of SEQ ID NO: 61;
(d) a light chain CDR1 having the amino acid sequence of SEQ ID NO: 62;
(e) a light chain CDR2 having the amino acid sequence of SEQ ID NO: 63; and
(f) a light chain CDR3 having the amino acid sequence of SEQ ID NO: 64, or
six complementarity determining regions (CDRs) comprising:
(a) a heavy chain CDR1 having the amino acid sequence of SEQ ID NO: 67;
(b) a heavy chain CDR2 having the amino acid sequence of SEQ ID NO: 68;
(c) a heavy chain CDR3 having the amino acid sequence of SEQ ID NO: 69;
(d) a light chain CDR1 having the amino acid sequence of SEQ ID NO: 70;
(e) a light chain CDR2 having the amino acid sequence of SEQ ID NO: 71; and
(f) a light chain CDR3 having the amino acid sequence of SEQ ID NO: 72, or
six complementarity determining regions (CDRs) comprising:
(a) a heavy chain CDR1 having the amino acid sequence of SEQ ID NO: 75;
(b) a heavy chain CDR2 having the amino acid sequence of SEQ ID NO: 76;
(c) a heavy chain CDR3 having the amino acid sequence of SEQ ID NO: 77;
(d) a light chain CDR1 having the amino acid sequence of SEQ ID NO: 78;
(e) a light chain CDR2 having the amino acid sequence of SEQ ID NO: 79; and
(f) a light chain CDR3 having the amino acid sequence of SEQ ID NO: 80, or
an antibody, antibody reagent, or antigen-binding portion thereof comprising the heavy chain variable region sequence of SEQ ID NO: 9, or
an antibody, antibody reagent, or antigen-binding portion thereof comprising the light chain variable region sequence of SEQ ID NO: 10, or
an antibody, antibody reagent, or antigen-binding portion thereof comprising the heavy chain variable region sequence of SEQ ID NO: 25, or
an antibody, antibody reagent, or antigen-binding portion thereof comprising the light chain variable region sequence of SEQ ID NO: 26, or
an antibody, antibody reagent, or antigen-binding portion thereof comprising the heavy chain variable region sequence of SEQ ID NO: 33, or
an antibody, antibody reagent, or antigen-binding portion thereof comprising the light chain variable region sequence of SEQ ID NO: 34, or
an antibody, antibody reagent, or antigen-binding portion thereof comprising the heavy chain variable region sequence of SEQ ID NO: 41, or
an antibody, antibody reagent, or antigen-binding portion thereof comprising the light chain variable region sequence of SEQ ID NO: 42, or
an antibody, antibody reagent, or antigen-binding portion thereof comprising the heavy chain variable region sequence of SEQ ID NO: 49, or
an antibody, antibody reagent, or antigen-binding portion thereof comprising the light chain variable region sequence of SEQ ID NO: 50, or
an antibody, antibody reagent, or antigen-binding portion thereof comprising the heavy chain variable region sequence of SEQ ID NO: 57, or
an antibody, antibody reagent, or antigen-binding portion thereof comprising the light chain variable region sequence of SEQ ID NO: 58, or
an antibody, antibody reagent, or antigen-binding portion thereof comprising the heavy chain variable region sequence of SEQ ID NO: 65, or
an antibody, antibody reagent, or antigen-binding portion thereof comprising the light chain variable region sequence of SEQ ID NO: 66, or
an antibody, antibody reagent, or antigen-binding portion thereof comprising the heavy chain variable region sequence of SEQ ID NO: 73, or
an antibody, antibody reagent, or antigen-binding portion thereof comprising the light chain variable region sequence of SEQ ID NO: 74; and
(ii) an immunosuppressive agent.

18. The composition, kit, or combination of claim 17, wherein the antibody, antibody reagent, or antigen-binding portion thereof is conjugated to the immunosuppressive agent.

19. A nucleic acid sequence encoding the antibody, antibody reagent, antigen-binding fragment thereof, or CAR of claim 1.

20. A cell comprising the antibody, antibody reagent, antigen-binding fragment thereof, or CAR of claim 1.

21. A pharmaceutical composition comprising the antibody, antibody reagent, antigen-binding fragment thereof, or CAR of claim 1.

22. A solid support comprising the antibody, antibody reagent, antigen-binding fragment thereof, or CAR of claim 1.

23.-24. (canceled)

25. A kit for the detection of PD1 polypeptide in a sample, the kit comprising at least a first antibody, antibody reagent, antigen-binding fragment thereof, or CAR of claim 1 immobilized on a solid support and comprising a detectable label.

26. A molecular complex comprising at least one antibody, antibody reagent, antigen-binding fragment thereof, or CAR of claim 1 bound to an PD1 polypeptide.

27. A method of treating an autoimmune disorder or an auto-inflammatory disorder in a subject in need thereof, the method comprising administering
(i) the antibody, antibody reagent, antigen-binding portion thereof, or CAR of claim 1 or an antibody, antibody reagent, antigen-binding portion thereof, or CAR comprising six complementarity determining regions (CDRs) comprising:
(a) a heavy chain CDR1 having the amino acid sequence of SEQ ID NO: 11;
(b) a heavy chain CDR2 having the amino acid sequence of SEQ ID NO: 12;
(c) a heavy chain CDR3 having the amino acid sequence of SEQ ID NO: 13;
(d) a light chain CDR1 having the amino acid sequence of SEQ ID NO: 14;
(e) a light chain CDR2 having the amino acid sequence of SEQ ID NO: 15; and
(f) a light chain CDR3 having the amino acid sequence of SEQ ID NO: 16, or
six complementarity determining regions (CDRs) comprising:
(a) a heavy chain CDR1 having the amino acid sequence of SEQ ID NO: 27;
(b) a heavy chain CDR2 having the amino acid sequence of SEQ ID NO: 28;
(c) a heavy chain CDR3 having the amino acid sequence of SEQ ID NO: 29;
(d) a light chain CDR1 having the amino acid sequence of SEQ ID NO: 30;
(e) a light chain CDR2 having the amino acid sequence of SEQ ID NO: 31; and
(f) a light chain CDR3 having the amino acid sequence of SEQ ID NO: 32, or
six complementarity determining regions (CDRs) comprising:
(a) a heavy chain CDR1 having the amino acid sequence of SEQ ID NO: 35;
(b) a heavy chain CDR2 having the amino acid sequence of SEQ ID NO: 36;
(c) a heavy chain CDR3 having the amino acid sequence of SEQ ID NO: 37;
(d) a light chain CDR1 having the amino acid sequence of SEQ ID NO: 38;
(e) a light chain CDR2 having the amino acid sequence of SEQ ID NO: 39; and
(f) a light chain CDR3 having the amino acid sequence of SEQ ID NO: 40, or
six complementarity determining regions (CDRs) comprising:
(a) a heavy chain CDR1 having the amino acid sequence of SEQ ID NO: 43;
(b) a heavy chain CDR2 having the amino acid sequence of SEQ ID NO: 44;
(c) a heavy chain CDR3 having the amino acid sequence of SEQ ID NO: 45;
(d) a light chain CDR1 having the amino acid sequence of SEQ ID NO: 46;
(e) a light chain CDR2 having the amino acid sequence of SEQ ID NO: 47; and
(f) a light chain CDR3 having the amino acid sequence of SEQ ID NO: 48, or
six complementarity determining regions (CDRs) comprising:
(a) a heavy chain CDR1 having the amino acid sequence of SEQ ID NO: 51;
(b) a heavy chain CDR2 having the amino acid sequence of SEQ ID NO: 52;
(c) a heavy chain CDR3 having the amino acid sequence of SEQ ID NO: 53;
(d) a light chain CDR1 having the amino acid sequence of SEQ ID NO: 54;
(e) a light chain CDR2 having the amino acid sequence of SEQ ID NO: 55; and
(f) a light chain CDR3 having the amino acid sequence of SEQ ID NO: 56, or
six complementarity determining regions (CDRs) comprising:
(a) a heavy chain CDR1 having the amino acid sequence of SEQ ID NO: 59;
(b) a heavy chain CDR2 having the amino acid sequence of SEQ ID NO: 60;
(c) a heavy chain CDR3 having the amino acid sequence of SEQ ID NO: 61;
(d) a light chain CDR1 having the amino acid sequence of SEQ ID NO: 62;
(e) a light chain CDR2 having the amino acid sequence of SEQ ID NO: 63; and
(f) a light chain CDR3 having the amino acid sequence of SEQ ID NO: 64, or
six complementarity determining regions (CDRs) comprising:
(a) a heavy chain CDR1 having the amino acid sequence of SEQ ID NO: 67;
(b) a heavy chain CDR2 having the amino acid sequence of SEQ ID NO: 68;
(c) a heavy chain CDR3 having the amino acid sequence of SEQ ID NO: 69;
(d) a light chain CDR1 having the amino acid sequence of SEQ ID NO: 70;
(e) a light chain CDR2 having the amino acid sequence of SEQ ID NO: 71; and
(f) a light chain CDR3 having the amino acid sequence of SEQ ID NO: 72, or
six complementarity determining regions (CDRs) comprising:
(a) a heavy chain CDR1 having the amino acid sequence of SEQ ID NO: 75;
(b) a heavy chain CDR2 having the amino acid sequence of SEQ ID NO: 76;
(c) a heavy chain CDR3 having the amino acid sequence of SEQ ID NO: 77;
(d) a light chain CDR1 having the amino acid sequence of SEQ ID NO: 78;
(e) a light chain CDR2 having the amino acid sequence of SEQ ID NO: 79; and
(f) a light chain CDR3 having the amino acid sequence of SEQ ID NO: 80, or
an antibody, antibody reagent, or antigen-binding portion thereof comprising the heavy chain variable region sequence of SEQ ID NO: 9, or
an antibody, antibody reagent, or antigen-binding portion thereof comprising the light chain variable region sequence of SEQ ID NO: 10, or
an antibody, antibody reagent, or antigen-binding portion thereof comprising the heavy chain variable region sequence of SEQ ID NO: 25, or
an antibody, antibody reagent, or antigen-binding portion thereof comprising the light chain variable region sequence of SEQ ID NO: 26, or
an antibody, antibody reagent, or antigen-binding portion thereof comprising the heavy chain variable region sequence of SEQ ID NO: 33, or
an antibody, antibody reagent, or antigen-binding portion thereof comprising the light chain variable region sequence of SEQ ID NO: 34, or
an antibody, antibody reagent, or antigen-binding portion thereof comprising the heavy chain variable region sequence of SEQ ID NO: 41, or
an antibody, antibody reagent, or antigen-binding portion thereof comprising the light chain variable region sequence of SEQ ID NO: 42, or
an antibody, antibody reagent, or antigen-binding portion thereof comprising the heavy chain variable region sequence of SEQ ID NO: 49, or
an antibody, antibody reagent, or antigen-binding portion thereof comprising the light chain variable region sequence of SEQ ID NO: 50, or
an antibody, antibody reagent, or antigen-binding portion thereof comprising the heavy chain variable region sequence of SEQ ID NO: 57, or
an antibody, antibody reagent, or antigen-binding portion thereof comprising the light chain variable region sequence of SEQ ID NO: 58, or
an antibody, antibody reagent, or antigen-binding portion thereof comprising the heavy chain variable region sequence of SEQ ID NO: 65, or
an antibody, antibody reagent, or antigen-binding portion thereof comprising the light chain variable region sequence of SEQ ID NO: 66, or
an antibody, antibody reagent, or antigen-binding portion thereof comprising the heavy chain variable region sequence of SEQ ID NO: 73, or
an antibody, antibody reagent, or antigen-binding portion thereof comprising the light chain variable region sequence of SEQ ID NO: 74;
to the subject.

28. The method of claim 27, wherein the autoimmune disorder or the autoinflammatory disorder is a T-cell mediated disorder.

29. The method of claim 27, wherein the autoimmune disorder or the autoinflammatory disorder is selected from the group consisting of:
type-1 diabetes, rheumatoid arthritis, multiple sclerosis, celiac disease, Rasmussen's encephalitis, hypothyroidism, Addison's disease, and amyotrophic lateral sclerosis (ALS).

30.-32. (canceled)

33. A method of suppressing an immune response or an inflammatory response in a subject in need thereof, the method comprising administering
(i) the antibody, antibody reagent, antigen-binding portion thereof, or CAR of claim 1 or an antibody, antibody reagent, antigen-binding portion thereof, or CAR comprising six complementarity determining regions (CDRs) comprising:
(a) a heavy chain CDR1 having the amino acid sequence of SEQ ID NO: 11;
(b) a heavy chain CDR2 having the amino acid sequence of SEQ ID NO: 12;
(c) a heavy chain CDR3 having the amino acid sequence of SEQ ID NO: 13;
(d) a light chain CDR1 having the amino acid sequence of SEQ ID NO: 14;
(e) a light chain CDR2 having the amino acid sequence of SEQ ID NO: 15; and
(f) a light chain CDR3 having the amino acid sequence of SEQ ID NO: 16, or
six complementarity determining regions (CDRs) comprising:
(a) a heavy chain CDR1 having the amino acid sequence of SEQ ID NO: 27;
(b) a heavy chain CDR2 having the amino acid sequence of SEQ ID NO: 28;
(c) a heavy chain CDR3 having the amino acid sequence of SEQ ID NO: 29;
(d) a light chain CDR1 having the amino acid sequence of SEQ ID NO: 30;
(e) a light chain CDR2 having the amino acid sequence of SEQ ID NO: 31; and
(f) a light chain CDR3 having the amino acid sequence of SEQ ID NO: 32, or
six complementarity determining regions (CDRs) comprising:
a) a heavy chain CDR1 having the amino acid sequence of SEQ ID NO: 35;
(b) a heavy chain CDR2 having the amino acid sequence of SEQ ID NO: 36;
(c) a heavy chain CDR3 having the amino acid sequence of SEQ ID NO: 37;
(d) a light chain CDR1 having the amino acid sequence of SEQ ID NO: 38;
(e) a light chain CDR2 having the amino acid sequence of SEQ ID NO: 39; and
(f) a light chain CDR3 having the amino acid sequence of SEQ ID NO: 40, or
six complementarity determining regions (CDRs) comprising:
(a) a heavy chain CDR1 having the amino acid sequence of SEQ ID NO: 43;
(b) a heavy chain CDR2 having the amino acid sequence of SEQ ID NO: 44;
(c) a heavy chain CDR3 having the amino acid sequence of SEQ ID NO: 45;
(d) a light chain CDR1 having the amino acid sequence of SEQ ID NO: 46;
(e) a light chain CDR2 having the amino acid sequence of SEQ ID NO: 47; and
(f) a light chain CDR3 having the amino acid sequence of SEQ ID NO: 48, or
six complementarity determining regions (CDRs) comprising:
(a) a heavy chain CDR1 having the amino acid sequence of SEQ ID NO: 51;
(b) a heavy chain CDR2 having the amino acid sequence of SEQ ID NO: 52;
(c) a heavy chain CDR3 having the amino acid sequence of SEQ ID NO: 53;
(d) a light chain CDR1 having the amino acid sequence of SEQ ID NO: 54;
(e) a light chain CDR2 having the amino acid sequence of SEQ ID NO: 55; and
(f) a light chain CDR3 having the amino acid sequence of SEQ ID NO: 56, or
six complementarity determining regions (CDRs) comprising:
(a) a heavy chain CDR1 having the amino acid sequence of SEQ ID NO: 59;
(b) a heavy chain CDR2 having the amino acid sequence of SEQ ID NO: 60;
(c) a heavy chain CDR3 having the amino acid sequence of SEQ ID NO: 61;
(d) a light chain CDR1 having the amino acid sequence of SEQ ID NO: 62;
(e) a light chain CDR2 having the amino acid sequence of SEQ ID NO: 63; and
(f) a light chain CDR3 having the amino acid sequence of SEQ ID NO: 64, or
six complementarity determining regions (CDRs) comprising:
(a) a heavy chain CDR1 having the amino acid sequence of SEQ ID NO: 67;
(b) a heavy chain CDR2 having the amino acid sequence of SEQ ID NO: 68;
(c) a heavy chain CDR3 having the amino acid sequence of SEQ ID NO: 69;
(d) a light chain CDR1 having the amino acid sequence of SEQ ID NO: 70;
(e) a light chain CDR2 having the amino acid sequence of SEQ ID NO: 71; and
(f) a light chain CDR3 having the amino acid sequence of SEQ ID NO: 72, or
six complementarity determining regions (CDRs) comprising:
(a) a heavy chain CDR1 having the amino acid sequence of SEQ ID NO: 75;
(b) a heavy chain CDR2 having the amino acid sequence of SEQ ID NO: 76;
(c) a heavy chain CDR3 having the amino acid sequence of SEQ ID NO: 77;
(d) a light chain CDR1 having the amino acid sequence of SEQ ID NO: 78;
(e) a light chain CDR2 having the amino acid sequence of SEQ ID NO: 79; and
(f) a light chain CDR3 having the amino acid sequence of SEQ ID NO: 80,
an antibody, antibody reagent, or antigen-binding portion thereof comprising the heavy chain variable region sequence of SEQ ID NO: 9, or
an antibody, antibody reagent, or antigen-binding portion thereof comprising the light chain variable region sequence of SEQ ID NO: 10, or
an antibody, antibody reagent, or antigen-binding portion thereof comprising the heavy chain variable region sequence of SEQ ID NO: 25, or
an antibody, antibody reagent, or antigen-binding portion thereof comprising the light chain variable region sequence of SEQ ID NO: 26, or
an antibody, antibody reagent, or antigen-binding portion thereof comprising the heavy chain variable region sequence of SEQ ID NO: 33, or
an antibody, antibody reagent, or antigen-binding portion thereof comprising the light chain variable region sequence of SEQ ID NO: 34, or
an antibody, antibody reagent, or antigen-binding portion thereof comprising the heavy chain variable region sequence of SEQ ID NO: 41, or
an antibody, antibody reagent, or antigen-binding portion thereof comprising the light chain variable region sequence of SEQ ID NO: 42, or
an antibody, antibody reagent, or antigen-binding portion thereof comprising the heavy chain variable region sequence of SEQ ID NO: 49, or
an antibody, antibody reagent, or antigen-binding portion thereof comprising the light chain variable region sequence of SEQ ID NO: 50, or
an antibody, antibody reagent, or antigen-binding portion thereof comprising the heavy chain variable region sequence of SEQ ID NO: 57, or
an antibody, antibody reagent, or antigen-binding portion thereof comprising the light chain variable region sequence of SEQ ID NO: 58, or
an antibody, antibody reagent, or antigen-binding portion thereof comprising the heavy chain variable region sequence of SEQ ID NO: 65, or
an antibody, antibody reagent, or antigen-binding portion thereof comprising the light chain variable region sequence of SEQ ID NO: 66, or
an antibody, antibody reagent, or antigen-binding portion thereof comprising the heavy chain variable region sequence of SEQ ID NO: 73, or
an antibody, antibody reagent, or antigen-binding portion thereof comprising the light chain variable region sequence of SEQ ID NO: 74;
to the subject.

34.-38. (canceled)

39. An antibody, antibody reagent, antigen-binding fragment thereof, or chimaeric antigen receptor (CAR), that specifically binds an PD1 polypeptide, said antibody reagent, antigen-binding portion thereof, or CAR comprising:
(a) a heavy chain CDR1 having the amino acid sequence of SEQ ID NO: 83;
(b) a heavy chain CDR2 having the amino acid sequence of SEQ ID NO: 84;
(c) a heavy chain CDR3 having the amino acid sequence of SEQ ID NO: 85;
(d) a light chain CDR1 having the amino acid sequence of SEQ ID NO: 86;
(e) a light chain CDR2 having the amino acid sequence of SEQ ID NO: 87; and
(f) a light chain CDR3 having the amino acid sequence of SEQ ID NO: 88.

40.-68. (canceled)
